(19) United States
(12) Patent Application Publication
(10) Pub. No.: US 2004/0259222 A1
(43) Pub. Date: Dec. 23, 2004

(54) Novel group of $g(a)-amylases and a method for identification and production of novel $g(a)-amylases

(76) Inventors: Roland Breves, Mettmann (DE); Beatrix Kottwitz, Erkrath (DE); Karl-Heinz Maurer, Erkrath (DE); Holger Zinke, Zwingenberg (DE); Jurgen Eck, Heppenhein (DE); Patrick Lorenz, Hirscberg (DE)

Correspondence Address: WOODCOCK WASHBURN LLP, ONE LIBERTY PLACE, 46TH FLOOR, PHILADELPHIA, PA, 19103, US

(21) Appl. No.: 10/482,076

(22) PCT Filed: Jun. 20, 2002
(86) PCT No.: PCT/EP02/06842

(30) Foreign Application Priority Data
Jun. 29, 2001 (DE) 101 31 441.8

(51) Int. Cl. C12N 9/32

(52) U.S. Cl. 435/204

(57) ABSTRACT

Described herein are a novel group of α-amylases belonging to a common sequence region as well as proteins with amylolytic function which are sufficiently similar to these α-amylases. Also described are methods for production of such enzymes, as well as various possible uses for such amylolytic proteins, in particular in detergents and cleaning agents. Also described herein is a PCR-based method for identifying and producing novel α-amylases from isolated nucleic acids, in particular from DNA isolated from collections of microorganisms, as well as particular primer oligonucleotides which may be used in the described method.

The present invention relates to a novel group of α-amylases belonging to a common sequence region and to proteins with amylolytic function which are sufficiently similar to said α-amylases, to methods for production thereof and to various possible uses for said amylolytic proteins, in particular in detergents and cleaning agents. The invention further relates to a PCR-based method for identifying and producing novel α-amylases from isolated genomic DNA, in particular from DNA isolated from collections of microorganisms, and to particular primer oligonucleotides and application thereof in said method.

α-Amylases (E.C. 3.2.1.1) hydrolyze α-1,4-glycosidic bonds of starch and starch-like polymers such as, for example, amylase, amylopectin or glycogen, which bonds are located inside the polymer, with the formation of dextrins and β-1,6-branched oligosaccharides. They are very much among the most important industrially utilized enzymes, for two reasons: on the one hand, like many substrate-degrading enzymes, they are usually released by microorganisms into the surrounding medium so that it is possible to obtain them on the industrial scale from the culture medium by fermentation and purification with comparatively little effort. On the other hand, amylases are required for a broad spectrum of applications.

First and foremost among the industrial uses of α-amylases is the production of glucose syrup. Other examples are the use as active components in detergents and cleaning agents, the use for treatment of raw materials in the manufacture of textiles, the use for producing adhesives or for producing sugar-containing food or food ingredients.

An example of an amylase which is particularly intensively used industrially is Bacillus licheniformis α-amylase which is supplied by Novo Nordisk A/S, Bagsvaerd, Denmark under the trade name Termamyl [symbol] . The amylase derived from B. subtilis and B. amyloliquefaciens, respectively, and disclosed in U.S. application U.S. Pat. No 1,227,374 is sold by the same company under the name BAN [symbol] .

This amylase molecule and its close relatives have been further developed in numerous inventions whose object was to optimize their enzymic properties for specific applications with the aid of various molecular-biological modifications. Such optimizations may relate, for example, to the substrate specificities, the stability of the enzyme under various reaction conditions or to the enzymic activity itself. Examples of such optimizations, which may be mentioned here, are the following applications: EP 0410498 for sizing textiles and WO 96/02633 for starch liquefaction.

Since developments which consist merely of optimizations of only a few known starting enzymes are possibly limited with respect to the achievable results, an intensive search for comparable enzymes for other natural sources is carried out in parallel. Starch-cleaving enzymes, for example from Pimelobacter, Pseudomonas and Thermus, have been identified for food production, cosmetics and pharmaceuticals (EP 0 636 693), and enzymes of the same type from Rhizobium, Arthrobacter, Brevibacterium and Micrococcus (EP 0 628 630), from Pyrococcus (WO 94/19454) and from Sulfolobus for starch liquefaction at elevated temperatures and strongly acidic reaction conditions (EP 0 727 485 and WO 96/02633), respectively. Bacillus sp. amylases (WO 95/26397 and WO 97/00324) have been found for the use at alkaline pH. Due to their low sensitivity to detergents, other amylases from various Bacilli (EP 0 670 367) are suitable for use in detergents or cleaning agents.

Further optimizations of the enzymes isolated from natural sources for the particular field of application may be carried out, for example, via molecular-biological methods (for example according to U.S. Pat. No. 5,171,673 or WO 99/20768) or via chemical modifications (DE 4013142). The patent application WO 99/43793, for example, describes a development of the known Novamyl α-amylase, in which sequence similarities between Novamyl [symbol] and known cyclodextrin glucanotransferases (CGTases) are utilized in order to construct a number of related molecules with the aid of molecular-biological techniques. Said molecules are α-amylases with additional CGTase-specific consensus sequences (boxes) and functions or, conversely, CGTases with additional regions and functions typical for α-amylases, or chimeras of the two molecules. The purpose of this development is to optimize Novamyl [symbol] for these applications.

The application WO 99/57250, for example, provides the teaching of linking enzymes suitable for the use in detergents and cleaning agents via chemical linkers to a binding domain which increases the effective enzyme concentration on the material to be cleaned.

A modern direction of enzyme development comprises combining elements of known proteins related to one another via random methods to give new enzymes having properties have not been obtained previously. Such methods are also listed under the generic term ‘directed evolution’ and include, for example the following methods: the StEP method (Zhao et al. (1998), Nat. Biotechnol., Volume 16, pp. 258-261), random priming recombination (Shao et al., (1998), Nucleic Acids Res., Volume 26, pp. 681-683), DNA shuffling (Stemmer, W. P. C. (1994), Nature, Volume 370, pp. 389-391) and RACHITT (Coco, W. M. et al. (2001), Nat. Biotechnol., Volume 19, pp. 354-359).

The recombination via methods of this kind requires the presence of sufficiently long regions on each of the nucleic acids used in order to achieve hybridization under the particular conditions. Starting sequences with identities which are more than 45% to one another at the amino acid level should be regarded as practicable for a successful hybridization and more than 50% for forcing the homologous recombination. At least two different sequences which are homologous to one another already define a sequence region which includes any novel sequences theoretically derivable from said starting sequences via recombination. For this purpose, they may also have homologies of less than 45%, if the nucleic acids derived therefrom can be made to recombine by any of the methods established in the prior art.

Despite all of these developments, however, there is the unchanged task of finding, in addition to the few natural amylolytic enzymes which are currently industrially utilized in unmodified form or in the form of further developments, further enzymes which a priori have a broad spectrum of applications and which may serve as starting points for application-specific further developments, in particular for random recombination methods.

The great genetic variety of the Gram-positive bacteria order of Actinomycetales, in particular of the genus Streptomyces, has hardly been investigated previously for amylolytic proteins suitable for industrial purposes. Only two Japanese patent applications are to be contemplated in this context. The application JP-A 62-143999 discloses an α-amylase from a representative of the genus Streptomyces, which is suitable for the use in detergents or cleaning agents. This organism, Streptomyces sp. KSM-9 or FERM P-7620, comes from a natural habitat and grows in alkaline medium. Said document describes the amylolytic enzyme merely via enzymic parameters and its suitability for the use in detergents and cleaning agents, but not via its DNA sequence or amino acid sequence.

The enzyme disclosed in the application JP-A 2000-60546 and derived from Streptomyces sp. TOTO-9805 or FERM BP-6359 has enzymic properties similar to those of the Streptomyces sp. KSM-9 enzyme. However, said application also characterizes said enzyme merely via enzymic parameters and not via the amino acid or nucleotide sequence. As a result of this, both amylases are available neither for heterologous expression and production nor for application-specific selection and optimization, since both traditional mutagenesis methods and directed evolution methods (EP-PCR, sequence shuffling, family shuffling) are based on the corresponding nucleic acid sequences.

The present invention is thus primarily based on the object of identifying natural α-amylases which have not been described previously and which themselves are suitable for possible industrial uses or which may serve as bases for application-specific further developments.

Preferably, this object should be considered as having been achieved by finding a plurality of α-amylases or partial sequences of a plurality of α-amylases, which are related to one another and can be homologized, since it is possible to derive a sequence region from such a homologization, which in turn can serve as starting point for generating further enzymes. The finding of α-amylase sequences as diverse as possible should be particularly advantageous, since this opens up a correspondingly broader sequence region with a corresponding multiplicity of possible variations; on the other hand, homology of the sequences obtained to one another should still be high enough in order to make a recombination via known methods possible. Preferably, at least some sequences should have homologies to one another of in each case from 50 to 60% identity at the amino acid level.

Part of the object was to obtain the nucleic acids coding for α-amylases of this kind, since said nucleic acids are essential both for the biotechnological production and for the further development of said enzymes.

Another part of the object was to find those organisms which naturally produce the α-amylases in question.

Another part of the object was to find a method by which such a pool of α-amylases can be provided.

As another part of the object, it should be possible to utilize the α-amylases, α-amylase fragments or α-amylase genes obtained or fragments thereof for finding or developing new enzymes.

Another part of the object was to make possible the biotechnological production of the α-amylases found or derivable α-amylases.

Another part of the object was to define possible industrial uses for the α-amylases found.

The first object is achieved by amylolytic proteins which are thus the first subject matter of the invention and whose amino acid sequences comprise a portion of which 98%, preferably 99%, particularly preferably 100%, are described by the consensus sequence of SEQ ID NO. 263, in particular via the subregion corresponding to positions 8 to 93. These include amylolytic proteins having the amino acid sequences indicated in the sequence listing under SEQ ID NO. 34 to 262, preferably the enzymes treated in the examples, in particular the enzymes derived from the species Streptomyces sp. B327* and B400B and enzymes which are sufficiently similar thereto or which can be derived therefrom by methods known per se. Preferred representatives can be isolated from natural organisms, in particular from those of the order Actinomycetales.

It is possible to derive from said sequences via homologization a sequence region which in turn can serve as starting point for generating further enzymes.

The invention secondly relates to nucleic acids coding for amylolytic proteins whose amino acid sequence comprises a portion of which 98%, preferably 99%, particularly preferably 100%, are described by the consensus sequence of SEQ ID NO. 263, in particular via the subregion corresponding to positions 8 to 93. These correspondingly preferably include the nucleic acids coding for the respective proteins of the first subject matter of the invention but also particular oligonucleotides which can be used in methods for finding such genes or gene fragments (see below).

The invention thirdly relates to the natural organisms containing nucleic acids coding for the proteins or protein fragments of the first subject matter of the invention. Particularly preferred embodiments thereof are the strains Streptomyces sp. 327* and Streptomyces sp. B400B which have been deposited under the numbers DSM 13990 and DSM 13991, respectively.

The invention fourthly relates to PCR-based methods for identifying and/or obtaining new amylases from a collection of organisms or nucleic acids, which methods are characterized in that PCR primers having in each case a variable 3′ region and a 5′ region highly homologous to regions of known amylases are used. Methods of this kind may be designed in various ways and be extended by optional process steps: these include sequencing the genes or gene fragments obtained, deriving peptides which may be characterized via immunochemical methods or via their biochemical properties. Further embodiments relate to the design of the primers with respect to the variability or selection of the regions from which they are derived; in particular, the primers used in the examples are preferred embodiments. Another possible embodiment relates to the origin of the material serving as PCR template, with collections of Actinomycetales being preferred. In further embodiments, the PCR products are studied via expression banks. Particular preference is given to a reaction process which leads to a multiplicity of similar products via which a sequence region can be defined.

The invention fifthly relates to the α-amylases, α-amylase fragments or α-amylase genes or fragments thereof, obtained by a method of the previous subject matter of the invention, for finding or developing new enzymes, by using said α-amylases, α-amylase fragments or α-amylase genes or fragments thereof either themselves for the screening for or development of novel primers or for the fusion or linkage to another protein or gene.

The invention sixthly relates to vectors having the nucleic acids of the second subject matter of the invention, to host cells transformed with such vectors and to all biotechnological methods for preparing a protein or derivative according to the first subject matter of the invention.

The invention seventhly relates to the possible industrial uses for the α-amylases found. These include detergents or cleaning agents which are characterized in that they comprise a protein or derivative according to the first subject matter of the invention, methods for starch liquefaction, in particular for producing ethanol, temporary bonding methods and various possible uses, in particular for the treatment of raw materials or intermediates in the manufacture of textiles, in particular for desizing cotton, for preparing linear and/or short-chain oligosaccharides, for hydrolyzing cyclodextrins, for liberating low-molecular weight compounds from polysaccharide supports or cyclodextrins, for preparing food and/or food ingredients, for preparing animal feed and/or animal feed ingredients and for dissolving starch-containing adhesive bonds.

A protein means in accordance with the present application a polymer which is composed of the natural amino acids, has a substantially linear structure and adopts usually a three dimensional structure to exert its function. In the present application, the 19 proteinogenic, naturally occurring L-amino acids are indicated by the internationally customary 1- and 3-letter codes.

An enzyme in accordance with the present application means a protein which exerts a particular biochemical function. Amylolytic proteins or enzymes with amylolytic function mean those which hydrolyze α-1,4-glycosidic bonds of polysaccharides, in particular those bonds located inside the polysaccharides, and which are also referred to as α-1,4-amylases (E.C. 3.2.1.1) or α-amylases for short.

Numerous proteins are formed as “preproteins”, i.e. together with a signal peptide. This then means the N-terminal part of the protein, whose function usually is to ensure the export of the produced protein from the producing cell into the periplasm or into the surrounding medium and/or the correct folding thereof. Subsequently, the signal peptide is removed from the remaining protein under natural conditions by a signal peptidase so that said protein exerts its actual catalytic activity without the initially present N-terminal amino acids. For example, the native α-amylase from Streptomyces sp. B327* is 461 amino acids in length, as shown in SEQ ID NO. 6. As illustrated in SEQ ID NO. 5, the signal peptide of this enzyme in comprises 30 amino acids so that the mature enzyme has a length of 431 amino acids.

Owing to their enzymic activity, preference is given for industrial applications to the mature peptides, i.e. the enzymes processed after their preparation, over the preproteins.

Pro-proteins are inactive precursors of proteins. Their precursors with signal sequence are referred to as pre-pro-proteins.

Nucleic acids mean in accordance with the present application the molecules which are naturally composed of nucleotides, serve as information carriers and code for the linear amino acid sequence in proteins or enzymes. They may be present as single strand, as a single strand complementary to said single strand or as double strand. For molecular-biological work, preference is given to the nucleic acid DNA as the naturally more durable information carrier. In contrast, an RNA is produced to implement the invention in a natural environment such as, for example, in an expressing cell, and RNA molecules essential to the invention are therefore likewise embodiments of the present invention.

In the case of DNA, the sequences of both complementary strands in in each case all three possible reading frames must be taken into account. The fact that different codon triplets can code for the same amino acids so that a particular amino acid sequence can be derived from a plurality of different nucleotide sequences which possibly have only low identity must also be taken into account (degeneracy of the genetic code). Moreover, various organisms differ in the use of these codons. For these reasons, both amino acid sequences and nucleotide sequences must be incorporated into the scope of protection, and nucleotide sequences indicated are in each case to be regarded only as coding by way of example for a particular amino acid sequence.

The information unit corresponding to a protein is also referred to as gene in accordance with the present application.

It is possible for a skilled worker, via nowadays generally known methods such as, for example, chemical synthesis or polymerase chain reaction (PCR) in combination with molecular-biological and/or protein-chemical standard methods, to prepare the appropriate nucleic acids up to complete genes on the basis of known DNA sequences and/or amino acid sequences. Such methods are known, for example, from the “Lexikon der Biochemie” [Encyclopedia of Biochemistry], Spektrum Akademischer Verlag, Berlin, 1999, Volume 1, pp. 267-271 and Volume 2, pp. 227-229.

Changes in the nucleotide sequence, as may be produced, for example, by molecular-biological methods known per se, are referred to as mutations. Depending on the type of change, deletion, insertion or substitution mutations or those in which various genes or parts of genes are fused to one another (shuffling) are known, for example; these are gene mutations. The corresponding organisms are referred to as mutants. The proteins derived from mutated nucleic acids are referred to as variants. Thus, for example, deletion, insertion, substitution mutations or fusions result in deletion-, insertion-, substitution-mutated or fusion genes and, at the protein level, to corresponding deletion, insertion or substitution variants, or fusion proteins.

Fragments mean all proteins or peptides which are smaller than natural proteins or than those proteins which correspond to completely translated genes, and which may, for example, also be obtained synthetically. Owing to their amino acid sequences, they may be related to the corresponding complete proteins. They may adopt, for example, identical structures or exert proteolytic activities or partial activities such as complexing of a substrate, for example. Fragments and deletion variants of starting proteins are in principle very similar; while fragments represent rather relatively small pieces, the deletion mutants rather lack only short regions and thus only individual partial functions.

At the nucleic acid level, the partial sequences correspond to fragments.

Chimeric or hybrid proteins mean in accordance with the present application those proteins which are composed of elements which naturally originate from different polypeptide chains from the same organism or from different organisms. This procedure is also called shuffling or fusion mutagenesis. The purpose of such a fusion may be, for example, to cause or to modify a particular enzymic function with the aid of the fused-to protein part. In accordance with the present invention, it is unimportant as to whether such a chimeric protein consists of a single polypeptide chain or of a plurality of subunits between which different functions may be distributed. To implement the latter alternative, it is possible, for example, to break down a single chimeric polypeptide chain into a plurality of polypeptide chains by a specific proteolytic cleavage, either posttranslationally or only after a purification step.

Proteins obtained by insertion mutation mean those variants which have been obtained via methods known per se by inserting a nucleic acid fragment or protein fragment into the starting sequences. They should be classified as chimeric proteins, due to their similarity in principle. They differ from the latter merely in the size ratio of the unaltered protein part to the size of the entire protein. In such insertion-mutated proteins the proportion of foreign protein is lower than in chimeric proteins.

Inversion mutagenesis, i.e. a partial sequence conversion, may be regarded as a special form of both deletion and insertion. The same applies to a regrouping of various molecule parts, which deviates from the original amino acid sequence. Said regrouping can be regarded as deletion variant, as insertion variant and as shuffling variant of the original protein.

Derivatives mean in accordance with the present application those proteins whose pure amino acid chain has been chemically modified. Those derivatizations may be carried out, for example, biologically in connection with protein biosynthesis by the host organism.

Molecular-biological methods may be employed here. However, said derivatizations may also be carried out chemically, for example by chemical conversion of an amino acid side chain or by covalent binding of another compound to the protein. Such a compound may also be, for example, other proteins which are bound, for example, via bifunctional chemical compounds to proteins of the invention. Such modifications may influence, for example, substrate specificity or the strength of binding to the substrate or cause transient blocking of the enzymic activity if the coupled-to substance is an inhibitor. This may be useful for the period of storage, for example. Likewise, derivatization means covalent binding to a macromolecular support.

Proteins may also be combined, via the reaction with an antiserum or a particular antibody, to groups of immunologically related proteins. The members of a group are distinguished in that they have the same antigenic determinant which is recognized by an antibody.

In accordance with the present invention, all enzymes, proteins, fragments and derivatives, unless they need to be explicitly referred to such, are included under the generic term ‘proteins’.

Vectors mean in accordance with the present invention elements which consist of nucleic acids and which contain a particular gene as characteristic nucleic acid region. They are capable of establishing said gene as a stable genetic element replicating independently of the remaining genome in a species or a cell line over several generations or cell divisions. Vectors are, in particular when used in bacteria, special plasmids, i.e. circular genetic elements. Genetic engineering distinguishes between, on the one hand, those vectors which are used for storage and thus, to a certain extent, also for genetic engineering work, the “cloning vectors”, and, on the other hand, those which perform the function of establishing the gene of interest in the host cell, i.e. enabling expression of the protein in question. These vectors are referred to as expression vectors.

Comparison with known enzymes which are deposited, for example, in generally accessible databases makes it possible to deduce the enzymic activity of an enzyme under consideration from the amino acid sequence or nucleotide sequence. Said activity may be modified qualitatively or quantitatively by other protein regions which are not involved in the actual reaction. This could relate to, for example, enzyme stability, activity, reaction conditions or substrate specificity.

Such a comparison is carried out by relating similar sequences in the nucleotide or amino acid sequences of the proteins under consideration to one another. This is called homologization. Relating the relevant positions to one another in the form of a table is referred to as alignment. When analyzing nucleotide sequences, again both complementary strands and in each case all three possible reading frames must be taken into account, likewise the degeneracy of the genetic code and the organism-specific codon usage. Meanwhile, alignments are produced by computer programs, for example by the FASTA or BLAST algorithms; this procedure is described, for example, by D. J. Lipman and W. R. Pearson (1985) in Science, Volume 227, pp. 1435-1441.

A compilation of all matching positions in the comparative sequences is referred to as consensus sequence.

Such a comparison also allows a statement about the similarity or homology of the comparative sequences to one another. This is expressed in percent identity, i.e. the proportion of identical nucleotides or amino acid residues at the same positions. A wider definition of the term homology also includes the conserved amino acid substitutions in this value. This is then referred to as percent similarity. Such statements may be made about whole proteins or genes or only about individual regions.

The generation of an alignment is the first step in defining a sequence space. This hypothetical space encompasses any sequences to be derived by permutation in individual positions, which result from taking into account all variations occurring in the relevant individual positions of said alignment. Each hypothetically possible protein molecule is a point in said sequence space. For example, two amino acid sequences which are substantially identical and have only at two different positions in each case two different amino acids thus create a sequence space of four different amino acid sequences. A very large sequence space is obtained if further sequences are found which are in each case homologous to individual sequences of a space. Such high homologies which exist in each case in pairs enable also sequences with very low homologies to be recognized as belonging to a sequence space.

Homologous regions of different proteins are those having the same functions which can be recognized by matches in the primary amino acid sequence. This ranges up to complete identities in very small regions, the “boxes”, which comprise only a few amino acids and usually exert functions essential for the overall activity. The functions of the homologous regions mean very small partial functions of the function exerted by the complete protein, such as, for example, the formation of individual hydrogen bonds for complexing a substrate or transition complex.

The term amylolytic protein of the invention thus means not only one having the pure function of hydrolyzing α-1,4-glycosidic bonds, which can be attributed to the few amino acid residues of a putative catalytically active site. The term additionally encompasses all functions supporting the hydrolysis of a α-1,4-glycosidic bond. Such functions may be achieved, for example, by individual peptides and by one or more individual parts of a protein by acting on the actually catalytically active regions. The term amylolytic function also encompasses only such modifying functions, since, on the one hand, it is not necessarily known exactly which amino acid residues of the protein of the invention actually catalyze the hydrolysis, and, on the other hand, particular individual functions may not be excluded definitively from the outset from involvement in the catalysis. The auxiliary functions or partial activities include, for example, binding of a substrate, of an intermediate or final product, activation or inhibition or mediation of a regulatory effect on the hydrolytic activity. This may also be, for example, the formation of a structural element located far away from the active site or a signal peptide whose function relates to exporting the produced protein out of the cell and/or to the correct folding thereof and without which usually no functional enzyme is produced in vivo. Overall, however, the result must be a hydrolysis of α-1,4-glycosidic bonds of starch or starch-like polymers.

In accordance with this, for example, the fragments of the α-amylases indicated in the sequence listing under SEQ ID NO. 34 to 262 are to be regarded as amylolytic proteins, since they are by nature components of larger proteins which overall are capable of hydrolyzing the α-1,4-glycosidic bonds of starch or starch-like polymers.

Within the scope of the present application, a distinction must be made between screening (hybridization screening or DNA screening) and activity assay. In general, the “screening” of transformants means a detection reaction suitable for identifying those clones in which the desired transformation event has taken place. It is usually geared, as, for example, in the case of the familiar blue-white selection, towards detection of a biochemical activity which the transformants have obtained or which is no longer present, after recombination has taken place. This type of biochemical detection reaction is referred to as activity assay in accordance with the present application.

Screening refers to the screening of a gene bank containing particular nucleic acids and the thereby possible identification of sufficiently similar nucleic acid sequences. This is carried out, for example, via Southern or Northern blot hybridizations, as are quite well known from the prior art. However, this term also includes, for example, PCR-based methods of the invention for identifying and/or obtaining new genes from a collection of organisms or nucleic acids, which methods are characterized in that PCR primers having a variable 3′ region and a 5′ region with high homology to corresponding regions of known genes are used. The performance of an enzyme means its efficacy in the industrial area considered in each case. Said performance is based on the actual enzymic activity but, in addition, depends on further factors relevant for the particular process. These include, for example, stability, substrate binding, interaction with the material carrying said substrate or interactions with other ingredients, in particular synergies. Thus, for example, the study of whether an enzyme is suitable for use in detergents or cleaning agents considers its contribution to the washing or cleaning performance of an agent formulated with further components. For various industrial applications, it is possible to further develop and optimize an enzyme via molecular-biological techniques known per se, in particular the abovementioned techniques.

The following microorganisms have been deposited according to the Budapest Treaty on the international recognition of the deposit of microorganisms from Apr. 28, 1977 with the Deutsche Sammlung von Mikroorganismen und Zellkulturen GmbH (DSMZ), Mascheroder Weg 1b in 38124 Braunschweig, Germany on Jan. 15, 2001: the isolate Actinomycetales/Streptomyces sp. B327* under accession number DSM 13990 and the isolate Actinomycetales/Streptomyces sp. B400B under accession number DSM 13991.

According to the present application, said microorganisms are in particular characterized in that they contain genes for α-amylases, whose complete DNA sequences and amino acid sequences are indicated in the sequence listing under SEQ ID NO. 5 and 6 and under SEQ ID NO. 7 and 8, respectively.

The object is achieved by providing according to the invention a multiplicity of α-amylases all of which are to be regarded as representatives of a particular sequence space which is defined by a partial sequence of said α-amylase, namely a portion of the (αβ)₈-barrel structural element known for amylases. Any amylolytic proteins whose amino acid sequence comprises a portion belonging to said specific sequence space and sufficiently similarly proteins are amylases of the invention.

Said sequence space is depicted in FIG. 3 and in the sequence listing under SEQ ID NO. 263 in two different ways, namely as abbreviations or combinations of all sequences found, showing a partial sequence of amylolytic proteins which comprises 100 positions. Sequence variations occur in 86 of these positions. Thus, for example, position 6 in the illustration of FIG. 3 is occupied by the amino acid X₀ which may be either isoleucine or leucine. The same information is provided by the sequence protocol in the lines preceding the consensus sequence, followed by the combination of all possible sequences to an artificial consensus sequence. It should be noted in particular that the amino acids of individual positions, for example in position 28, may be occupied by a single or else a sequence of two or more amino acids.

The consensus sequence shown has only very low variance in positions 1-7 and 94-100, which results from the PCR-based method developed for identifying this sequence, which is further described below and in the examples: these partial sequences correspond to the DNA regions to which the primers used for amplification have bound. Depending on the stringency of the conditions under which this binding takes place, deviating nucleotide sequences may also be bound and amplified; with comparatively low selectivity, the DNA product obtained thus does not completely correspond to the template at these sites. For this reason, the scope of protection is directed in particular towards the subregion corresponding to positions 8 to 93 of the consensus sequence shown.

This consensus sequence which describes the sequence space is based on 231 homologizable partial sequences of α-amylases, which are depicted in the sequence listing under SEQ ID NO. 2, 4 and SEQ ID NO. 34 to 262 and which define the abovementioned sequence space by their variations in defined individual positions.

The derivation of a consensus sequence of this kind will be illustrated below; technical details can in each case be found in the examples of the present application.

The sequences of α-amylases (E.C. 3.2.1.1), for example of Gram-positive eubacteria of various genera of the order Actinomycetales (Streptomyces, Thermomonospora etc.), may be obtained from generally accessible databases. Comparison thereof, for example via producing an alignment, allows identification of sequence regions which are conserved between the species. A broad sequence space is obtained by detecting those conserved regions which flank variable sequence regions. These conserved regions are also referred to as sequence anchors, since the variable sequence regions become available via them. The blocks A to E highlighted in FIG. 1 were identified as conserved sequence blocks. It is possible to derive from their amino acid sequences or, rather better, from their nucleotide sequences PCR primers which, as forward and reverse primers, should be directed toward each other in such a way that a PCR in each case comprises the variable region.

Suitable templates for the PCR may be genomic DNA preparations of known, but also unknown, bacteria isolates, for example from soil samples. They should be available in pure culture to provide afterwards pure PCR products and pure enzymes. Samples of this kind may be taken simply from nature and cultured by applying particular conditions (e.g. pH, aeration, utilizable substrate, presence of otherwise toxic compounds, incidence of light, etc.) in which appropriate unicellular organisms grow which may then be isolated with methods known per se and further cultured under the appropriate conditions.

The following articles provide an overview over methods for isolating actinomycetes and streptomycetes:

Nüesch (1965): “Isolierung und Selektionierung von Actinomyceten”; Zbl. Bakt. I., Supplement 1, pp. 234-252;

Williams & Cross (1971): “Actinomyces”; in: Norris, J. R., Ribbons, D. W. (Editor) Methods in Microbiology, Acad. Press, London, Volume 4, pp. 295-334;

Williams & Wellington (1982): “Principles and Problems of selective Isolation of Microbes”, in: Bullock, J. D., Nisbet, L. J., Win-stanley, D. J. (Editor), “Bioactive Microbial Products: Search and Discovery”, Acad. Press London, pp. 9-26; and

Wellington & Cross (1983): “Taxonomy of antibiotic-producing actinomyces and new approaches for their selective isolation”, Progr. Industr. Microbiol., Volume 17, pp. 7-36.

In the example, an appropriate collection of Actinomycetales isolates having Streptomyces properties was used. In this example, the isolation had been carried out starting from soil samples with addition of the antibiotic nystatin to suppress the accompanying flora.

Positive results were obtained from the corresponding PCR mixtures, in particular with the primer combination GEX024 (forward)/GEX026 (SEQ ID NO. 9, and 10, respectively) which have been derived from the sequence regions C (GEX024) and D (GEX026) (FIG. 1) and correspond to the amylase domains β4 and β7 of the (αβ)₈ barrel structure, as defined in the article “Alpha-Amylase family: molecular biology and evolution” (Janecek, S. (1997), Prog. Biophys. Mol. Biol. 67 (1), pp. 67-97). Said primers produced PCR products of approx. 300 bp in length of all isolates assayed.

Said PCR products were sequenced. The deduced amino acid sequences are listed in the sequence listing under SEQ ID NO. 2, 4 and 34 to 262. A comparison of these partial sequences with the entries in the GenBank enzyme database (National Center for Biotechnology Information NCBI, National Institutes of Health, Bethesda, Md., USA) confirms that all of these partial sequences are amylase partial sequences. The result of this comparison is depicted in table 1 which also indicates in each case the most similar database entries for the partial sequences found and the degree of homology between these two sequences.

TABLE 1
List of 231 individual sequences and % identity to
their closest relatives in GenBank (NCBI; Release 121.0);
determined by the FASTA program on 2.2.2001.

| | SEQ ID NO. | | |
|---|---|---|---|
| | according | Closest | |
| Streptomyces sp. | to sequence | database | Identity |
| . . . | listing | hit | (%) |
| B327* | 2 | Y13332 | 92 |
| B400B | 4 | M15540 | 74.2 |
| B1002 | 34 | AL352956 | 81 |
| B1003B | 35 | AL352956 | 80 |
| B1006 | 36 | M15540 | 85.6 |
| B1008A1 | 37 | U51129 | 77 |
| B1009A | 38 | Y13332 | 88 |
| B1010 | 39 | Y13332 | 88 |
| B1011 | 40 | Y13332 | 90 |
| B1012B | 41 | Y13332 | 89 |
| B1014A1 | 42 | Y13332 | 91 |
| B1017C | 43 | Y13332 | 91 |
| B1019 | 44 | Y13332 | 90 |
| B101A | 45 | Z85949 | 83 |
| B101B | 46 | Y13332 | 88 |
| B102 | 47 | M18244 | 64 |
| B1020C | 48 | U51129 | 96 |
| B1022A | 49 | Y13332 | 85 |
| B1028 | 50 | Z85949 | 74 |
| B1029 | 51 | AL352956 | 80 |
| B1030A | 52 | Z85949 | 81 |
| B1035B | 53 | M25263 | 74 |
| B1036 | 54 | Y13332 | 85 |
| B1037A | 55 | M25263 | 82 |
| B1039A | 56 | AL352956 | 86 |
| B103A | 57 | Z85949 | 82 |
| B1041A1 | 58 | Y13332 | 91 |
| B1043A | 59 | AL352956 | 97 |
| B1044C | 60 | AL352956 | 75 |
| B1045 | 61 | Y13332 | 91 |
| B1046A | 62 | AL352956 | 90 |
| B1047A1 | 63 | Z85949 | 97 |
| B1048A | 64 | AL352956 | 96 |
| B1049A | 65 | AL352956 | 86 |
| B1050A | 66 | Z85949 | 85 |
| B1052A2 | 67 | AL352956 | 88 |
| B1053 | 68 | M18244 | 97 |
| B1059 | 69 | M15540 | 75.3 |
| B1060 | 70 | M25263 | 80 |
| B1061B | 71 | Z85949 | 80 |
| B1065 | 72 | M25263 | 79 |
| B1067A | 73 | AL352956 | 81 |
| B1068 | 74 | M25263 | 79 |
| B1069B | 75 | M18244 | 92 |
| B106C | 76 | Y13332 | 89 |
| B107 | 77 | U51129 | 87 |
| B1070A | 78 | Y13332 | 92 |
| B1071 | 79 | M25263 | 79 |
| B1072A | 80 | Y13332 | 94 |
| B108 | 81 | M25263 | 92 |
| B109A | 82 | Y13332 | 86 |
| B114C | 83 | Y13332 | 88 |
| B115 | 84 | Y13332 | 90 |
| B117A1 | 85 | Y13332 | 88 |
| B118 | 86 | M25263 | 80 |
| B119E | 87 | U51129 | 75 |
| B120alt | 88 | Y13332 | 88 |
| B123 | 89 | M18244 | 92 |
| B124 | 90 | Y13332 | 89 |
| B125C | 91 | Z85949 | 89 |
| B126A | 92 | Y13332 | 86 |
| B127A | 93 | Y13332 | 85 |
| B128B | 94 | U51129 | 85 |
| B130B | 95 | M25263 | 93 |
| B131 | 96 | M25263 | 92 |
| B134 | 97 | M25263 | 92 |
| B135A | 98 | Y13332 | 86 |
| B137 | 99 | M25263 | 78 |
| B138 | 100 | U51129 | 85 |
| B138A | 101 | U51129 | 85 |
| B138A2 | 102 | U51129 | 86 |
| B140 | 103 | M25263 | 92 |
| B141 | 104 | M25263 | 92 |
| B142 | 105 | X57568 | 77 |
| B143 | 106 | M25263 | 92 |
| B148A | 107 | Y13332 | 88 |
| B152A | 108 | M25263 | 82 |
| B153(B) | 109 | AL352956 | 89 |
| B154A | 110 | Y13332 | 87 |
| B156B | 111 | AL352956 | 89 |
| B157C | 112 | Y13332 | 88 |
| B158A | 113 | Y13332 | 89 |
| B159 | 114 | Y13332 | 89 |
| B160B | 115 | Y13332 | 88 |
| B161A | 116 | X57568 | 86 |
| B166B | 117 | U51129 | 89 |
| B168 | 118 | Y13332 | 88 |
| B179 | 119 | M25263 | 82 |
| B181C | 120 | Y13332 | 89 |
| B183B | 121 | X57568 | 97 |
| B184 | 122 | M18244 | 96 |
| B185(B) | 123 | Y13332 | 88 |
| B186A | 124 | Y13332 | 83 |
| B187A | 125 | AL352956 | 88 |
| B187A2 | 126 | AL352956 | 88 |
| B194A | 127 | Z85949 | 62 |
| B194B1 | 128 | Y13332 | 87 |
| B196A2C | 129 | Y13332 | 87 |
| B196B | 130 | Y13332 | 87 |
| B197B | 131 | Y13332 | 87 |
| B198C2 | 132 | Y13332 | 92 |
| B200B | 133 | Y13332 | 88 |
| B201A | 134 | Y13332 | 87 |
| B202A | 135 | Y13332 | 87 |
| B202B | 136 | Y13332 | 88 |
| B206A | 137 | Y13332 | 87 |
| B207 | 138 | Y13332 | 90 |
| B208B | 139 | Z85949 | 85 |
| B209B | 140 | Y13332 | 87 |
| B210 | 141 | Z85949 | 89 |
| B211A | 142 | Y13332 | 88 |
| B212B1 | 143 | Y13332 | 88 |
| B213 | 144 | Y13332 | 86 |
| B214B | 145 | AL352956 | 79 |
| B214C | 146 | M25263 | 83 |
| B215 | 147 | Y13332 | 88 |
| B218D2 | 148 | Y13332 | 87 |
| B219A | 149 | Y13332 | 88 |
| B220B | 150 | Y13332 | 88 |
| B221A | 151 | Y13332 | 88 |
| B222(B) | 152 | Z85949 | 88 |
| B223A | 153 | U51129 | 88 |
| B224(A) | 154 | Y13332 | 87 |
| B225B | 155 | AL352956 | 73 |
| B226B | 156 | AL352956 | 88 |
| B227B2 | 157 | Z85949 | 76 |
| B228B | 158 | U51129 | 88 |
| B230B1 | 159 | AL352956 | 75 |
| B231A | 160 | Z85949 | 74 |
| B233C | 161 | Z85949 | 76 |
| B234 | 162 | Y13332 | 88 |
| B235A | 163 | Z85949 | 76 |
| B237A | 164 | Y13332 | 89 |
| B238A | 165 | Y13332 | 89 |
| B240A | 166 | Y13332 | 87 |
| B241B2 | 167 | Y13332 | 88 |
| B242A1 | 168 | M25263 | 78 |
| B243C | 169 | U51129 | 86 |
| B244B2 | 170 | AL352956 | 75 |
| B246 | 171 | Y13332 | 89 |
| B247A | 172 | Z85949 | 74 |
| B248B2 | 173 | AL352956 | 81 |
| B249A | 174 | AL352956 | 75 |
| B249C | 175 | U51129 | 77 |
| B250A2 | 176 | Y13332 | 87 |
| B251B | 177 | Y13332 | 88 |
| B252A | 178 | U51129 | 86 |
| B253 | 179 | Y13332 | 89 |
| B253A | 180 | U51129 | 87 |
| B255B2 | 181 | Y13332 | 89 |
| B256A | 182 | Y13332 | 88 |
| B259A | 183 | Y13332 | 88 |
| B261 | 184 | U51129 | 96 |
| B278 | 185 | Z85949 | 87 |
| B279 | 186 | Y13332 | 88 |
| B280C | 187 | Y13332 | 88 |
| B284A | 188 | Y13332 | 86 |
| B286A | 189 | Y13332 | 87 |
| B287 | 190 | M25263 | 81 |
| B292A | 191 | M25263 | 95 |
| B3001org | 192 | Y13332 | 84 |
| B3002org | 193 | Z85949 | 74 |
| B3003org | 194 | Y13332 | 88 |
| B3017 | 195 | X57568 | 77 |
| B306 | 196 | Y13332 | 84 |
| B308 | 197 | Y13332 | 87 |
| B311 | 198 | M25263 | 81 |
| B315 | 199 | Y13332 | 87 |
| B317 | 200 | X57568 | 82 |
| B318 | 201 | M25263 | 76 |
| B319 | 202 | Y13332 | 87 |
| B320A | 203 | Y13332 | 89 |
| B321 | 204 | M25263 | 78 |
| B322A | 205 | Y13332 | 89 |
| B323 | 206 | M25263 | 83 |
| B326 | 207 | Y13332 | 84 |
| B327B | 208 | X57568 | 94 |
| B335org | 209 | M25263 | 83 |
| B345 | 210 | Y13332 | 88 |
| B346 | 211 | Y13332 | 91 |
| B347 | 212 | X59159 | 45.2 |
| B348 | 213 | Y13332 | 85 |
| B350 | 214 | Y13332 | 90 |
| B352 | 215 | Y13332 | 90 |
| B353 | 216 | Y13332 | 76 |
| B354 | 217 | Y13332 | 87 |
| B355 | 218 | Y13332 | 87 |
| B356 | 219 | Y13332 | 92 |
| B357 | 220 | X57568 | 75 |
| B358 | 221 | M25263 | 80 |
| B359 | 222 | Y13332 | 89 |
| B360 | 223 | Y13332 | 86 |
| B361 | 224 | M18244 | 44.2 |
| B362 | 225 | Y13332 | 87 |
| B363org | 226 | U51129 | 86 |
| B366 | 227 | Y13332 | 89 |
| B368 | 228 | Y13332 | 86 |
| B370 | 229 | AL352956 | 80 |
| B371 | 230 | Y13332 | 92 |
| B372 | 231 | Y13332 | 88 |
| B373 | 232 | Y13332 | 88 |
| B374B | 233 | Y13332 | 89 |
| B375 | 234 | Y13332 | 89 |
| B376 | 235 | Y13332 | 91 |
| B380 | 236 | Y13332 | 87 |
| B382 | 237 | Y13332 | 85 |
| B390 | 238 | M25263 | 83 |
| B392A | 239 | AL352956 | 97 |
| B393 | 240 | Z85949 | 74 |
| B394 | 241 | Z85949 | 76 |
| B395 | 242 | Y13332 | 88 |
| B396(A) | 243 | Y13332 | 90 |
| B400 | 244 | Y13332 | 88 |
| B4006 | 245 | Y13332 | 89 |
| B4006B | 246 | X59159 | 94.2 |
| B400A | 247 | Y13332 | 88 |
| B400A2 | 248 | Y13332 | 92 |
| B400B3 | 249 | M25263 | 80 |
| B400C | 250 | X57568 | 94 |
| B400C2 | 251 | M18244 | 91 |
| B400D | 252 | M25263 | 89 |
| B400D2 | 253 | M25263 | 89 |
| B400E | 254 | M25263 | 79 |
| B400G | 255 | Y13332 | 87 |
| B400G2 | 256 | Y13332 | 87 |
| B400I | 257 | M25263 | 81 |
| B400J | 258 | X59159 | 45.7 |
| B400K | 259 | X59159 | 45.7 |
| B400L | 260 | X59159 | 45.7 |
| B402 | 261 | M25263 | 90 |
| B907 | 262 | M25263 | 80 |

The comparison with the sequences deposited in the database shows that the most similar proteins have homologies of 97% identity. In the case of Streptomyces sp. B1053 α-amylase, the most similar protein is the enzyme with accession number M18244, which is the α-amylase of Streptomyces limosus, in the case of the enzymes of Streptomyces sp. B1043A and B392A, it is the enzyme with accession number AL352956 (α-amylase of Streptomyces coelicolor A3(2)) and, in the case of Streptomyces sp. B1047A1 α-amylase, it is the enzyme with the accession number Z85949, which is the α-amylase B of Streptomyces lividans.

An alignment of these 231 sequences is possible; in the present example one such alignment was produced with the aid of the Clustal X® program, version 1.64b (standard settings; described in: Thompson, J. D., Higgins, D. G. and Gibson, T. J. (1994), “CLUSTAL W: improving the sensitivity of progressive multiple sequence alignment through sequence weighting, position specific gap penalties and weight matrix choice”, Nucleic Acids Res., Volume 22, pages 4673-4680). As already indicated above, the consensus sequence can be found in FIG. 3 and SEQ ID NO. 263 in two alternative representations.

This consensus sequence, and therefore the corresponding sequence space defined by the variable amino acids, are therefore based on 231 individual sequences which were obtained from natural sources. Said sequence space comprises, by calculation, approx. 10⁵¹ different amino acid sequences all of which are described by said consensus sequence. Despite this large number, there are up until now no known amylases having a homologous region which is more than 97% identical to any of these sequences.

This consensus sequence thus defines an entirely new group of α-amylases. It indicates a region which is located between the two conserved regions C and D depicted in FIG. 1. This region is a secondary structural element, namely the domains β4 and β7 of the (αβ)₈ barrel structure, as defined in the article by S. Janecek (1997) in Prog. Biophys. Mol. Biol. 67 (1), pp. 67-97, and is regarded as being characteristic and a necessary structural element for the enzyme family of α-amylases, since it ensures optimal spatial arrangement of the other functional part of the molecule, in particular of the active site.

Independently thereof, however, a different rearrangement of domains is also conceivable for newly found or generated sequences. Therefore, all those α-amylases which contain at any position a partial sequence which can be described by the consensus sequence of SEQ ID NO. 263 are also embodiments of the present invention.

The amino acid sequences inside said sequence space are identical within the desired order of magnitude. Thus the two partial sequences of the α-amylases of Streptomyces sp. B327* and B400B are homologous to one another with 57% identity at the amino acid level. Overall, the minimum value determined for two of the 231 individual sequences determined was 32% identity and the maximum value was 99% identity at the amino acid level. Thus, each of the sequences found is at least 32% identical to any other sequence within said sequence space. Some values are substantially higher, as can also be estimated on the basis of the values in table 1, since frequently the same known proteins were found to be most similar to the various sequences.

Homologies of more than 30% at the amino acid level between the sequences used are regarded as being required for methods of obtaining new enzymes via random recombination (e.g., according to Zhao et al. (1998), Nat. Biotechnol., Volume 16, pp. 258-261, Shao et al., (1998), Nucleic Acids Res., Volume 26, pp. 681-683 or Stemmer, W. P. C. (1994), Nature, Volume 370, pp. 389-391) and values of more than 45% are advantageous. In providing now this sequence space, in particular its actually disclosed representatives, a pool of related sequences with sufficient sequence homology is provided which makes possible further application-relevant optimization by directed evolution. The diversity of the comprised sequences, on the other hand, suggests varying amylolytic properties with respect to, for example, optimal temperatures or stability to external influences.

Any molecules belonging to said sequence space or groups therefrom, sequence subspaces so to speak, may be selected for such methods. This depends on how many variants are to be generated or whether only particular subregions are to be varied. Two of the sequences belonging to said space already define a separate sequence (sub)space.

Since for the consensus sequence sequences of two or more amino acids are allowed in individual positions such as, for example, that of amino acid 28, and since other positions such as 45 or 70 may also remain unoccupied, homologies may also result within this consensus sequence defined by 100 amino acid positions, whose percentages are not integers.

For this reason, any amylolytic proteins whose amino acid sequence comprises a portion of which 98% and, increasingly preferably, 98.25%, 98.5%, 98.75%, 99%, 99.25%, 99.5%, 99.75%, and particularly preferably 100%, are defined by the consensus sequence of SEQ ID NO. 263 are claimed according to the invention. As setforth above, this applies in particular to the subregion corresponding to positions 8 to 93.

This argumentation applies particularly to those amylolytic proteins whose amino acid sequence comprises a portion of which 98% and, increasingly preferably, 98.25%, 98.5%, 98.75%, 99%, 99.25%, 99.5%, 99.75%, and particularly preferably 100%, are identical to any of the amino acid sequences indicated in SEQ ID NO. 34 to SEQ ID NO. 262, in particular across the subregion corresponding to positions 8 to 93 according to the consensus sequence of SEQ ID NO. 263.

For the relevant partial sequences have been determined for said proteins in the above-described manner and are provided by the sequence listing. The result of the database search, which is depicted in table 1, was that there is up until now no known α-amylase which contains any of the partial sequences indicated. The highest homology found was 97% (see above). These amino acid sequences define a sequence space which is also included within the scope of protection. Thus each point of said sequence space is a partial sequence of the invention and a solution of the prescribed object.

In particular, those enzymes which carry conserved substitutions [lacuna] either or both of the apparently highly conserved positions 11 and 75 which are occupied throughout with histidine and leucine, respectively, in the consensus sequence are included within the scope of protection. Said substitutions include any of the basic amino acids lysine, arginine or proline for the histidine and/or any of the aliphatic amino acids glycine, alanine, valine or isoleucine for the leucine.

The α-amylases of said sequence space are characterized in that said genes coding for said α-amylases can be used as templates in a PCR together with the primer pair GEX024/GEX026 to amplify fragments which can be defined by the consensus sequence of SEQ ID NO. 263 and which are identical to any of the partial sequences indicated in the sequence listing. Owing to the nature of the PCR, the subregions from positions 8 to 93 are particularly characteristic for the fragments in question.

This was shown in example 1 for the following Streptomyces sp. strains: B101A, B114C, B134, B135A, B138A, B152A, B153(B), B156B, B157C, B158A, B160B, B161A, B373, B375, B380, B390, B392A and B394. Sequencing of the PCR products of the genomic DNA of said Actinomycetales isolates from various natural habitats and deduction of the corresponding amino acid sequence resulted in the sequences which are listed in the sequence listing under the following numbers: 45, 83, 97, 98, 101, 108, 109, 111, 112, 113, 115, 116, 232, 234, 236, 238, 239 and 241.

As table 1 reveals, the amylase of the strain Streptomyces sp. B392A has the highest degree of homology of the amylases listed here to a known α-amylase in the region of the consensus sequence. Said homology is 97% to the Streptomyces coelicolor A3(2) α-amylase (database entry AL352956). These amino acid sequences define a sequence space which is subspace of the sequence space defined by the consensus sequence of SEQ ID NO. 263 and which is included within the preferred scope of protection. Thus, each point of said sequence space is a partial sequence of the invention and a preferred solution of the prescribed object.

In other words, any α-amylases are claimed whose amino acid sequences can be homologized in a subregion with the consensus sequence of SEQ ID NO. 263, having in said region only those amino acids which are located at the corresponding position in any of these three sequences. In accordance with this, they may be traced back to any of said sequences in each position of said portion.

For this reason, any amylolytic proteins whose amino acid sequence comprises a portion which is 98% and, increasingly, preferably, 98.25%, 98.5%, 98.75%, 99%, 99.25%, 99.5%, 99.75%, and particularly preferably 100%, identical to any of said sequences or which can be traced back directly to any of said sequences in each homologous position are claimed according to the invention. As set forth above, this applies in particular across the subregion corresponding to positions 8 to 93.

In the examples, the α-amylases of the strains Streptomyces sp. B327*, B400B, and B327 are studied in more detail. The subregions of these amylases, which correspond to the consensus sequence (SEQ ID NO. 263) are depicted in the sequence listing under SEQ ID NO. 2, 4 and 208. Table 1 reveals that the Streptomyces sp. B327B α-amylase has the highest degree of homology to any of these amylases, as determined by a database search. Said degree of homology is 94% identity to the Streptomyces griseus α-amylase (database entry X57568).

Said amino acid sequences define a sequence space which is a subspace of the sequence space as defined by the consensus sequence of SEQ ID NO. 263 and which is included within the particularly preferred scope of protection. Thus, each point of the sequence space defined by said sequences is a partial sequence of the invention and a particularly preferred solution of the prescribed object. They can be derived by using, via an alignment across the region corresponding to the consensus sequence of SEQ ID NO. 263, in the sequential amino acid positions in each case those amino acids which are located at the same site in any of the starting sequences mentioned.

For this reason, any amylolytic proteins whose amino acid sequence comprises a portion which is 95% and, increasingly preferably, 96%, 96.5%, 97%, 97.5%, 98%, 98.5%, 99%, 99.5%, and particularly preferably 100%, identical to any of these sequences are claimed according to the invention. As set forth above, this applies in particular across the subregion corresponding to positions 8 to 93. The α-amylases of the strains Streptomyces sp. B327* and B400B are homologous to one another with 57% identity over the entire length of their amino acid sequence, as can be gaged from the alignment of the FIG. 5, for example. This value makes them appear particularly suitable for random methods for generating new sequences for amylolytic proteins.

These two amylolytic enzymes define, via their complete amino acid sequences indicated also in SEQ ID NO. 6 and 8, respectively a separate sequence space corresponding to the complete proteins. Further members of this space which can be derived from these two sequences by recombination of the amino acids prescribed in the relevant positions likewise achieve the object. They can be deduced via an alignment corresponding to FIG. 5 by each of said positions being occupied by an amino acid located at the homologous site in either of the two starting sequences.

According to the invention, conserved substitutions should also be possible here. These include substitutions within the following amino acid groups:

aliphatic amino acids: G, A, V, L, I;

sulfur-containing amino acids: C, M;

aromatic amino acids: F, Y, W;

hydroxyl group-containing amino acids: S, T;

acid amide group-containing amino acids: N, Q;

acidic amino acids: D, E;

basic amino acids: H, K, R, P.

Preference is given to those amino acid positions which can be traced back directly to either of the two starting sequences, i.e. which are identical to either of the two prescribed amino acids, over said conserved substitutions.

Thus, any amylolytic proteins whose amino acid sequences can be traced back via a conserved substitution, preferably directly, in each individual homologous position to either of the two sequences of Streptomyces sp. B327* and B400B are included within the scope of protection of the present invention.

The object given is preferably achieved by amylolytic proteins having an amino acid sequence which is at least 93% identical to the amino acid sequence indicated in SEQ ID NO. 6, preferably in positions 31 to 461, particularly preferably in positions 210-300. Said amino acid sequence is that of the α-amylase of the invention from the Streptomyces sp. B327* Actinomycetales isolate.

Example 4 of the present application illustrates in detail how to obtain such a sequence containing a complete gene. To this end, an expression gene bank is conveniently prepared, i.e. the genomic DNA of the starting organisms in which the special gene is to be found is fragmented and expressed in isolated host organisms.

Genes, at least α-amylase genes of Actinomycetales, may be cloned and isolated by carrying out expression cloning in the heterologous host organism Escherichia coli, as example 4 shows. According to Horinouchi et al. (Horinouchi, S. Uouzumi, T. Beppu, T. (1980): “Cloning of Streptomyces DNA into Escherichia coli: Absence of Heterospecific Gene Expression of Streptomyces Genes in E. coli”; Agric. Biol. Chem., Volume 44 (2), pp. 367-381) a heterologous recognition of the promoters from actinomycetes in the E. coli host organism cannot be assumed. Thus it is advisable to use externally inducible promoters, for example the β-Galactosidase promoter of the E. coli lac operon (lac promoter) which is inducible by IPTG (isopropylthiogalactoside). Thus, addition of IPTG can induce expression of the genes under the control of said promoter in host strains having a lacIq genotype (e.g. JM109).

The E. coli strains JM 109, DH 10B and DH 12S proved suitable for cloning the α-amylases derived from Actinomycetales and for the activity-dependent detection thereof, since, although they have the periplasmic enzymes encoded by maIS (Freundlieb, S., Boos, W. (1986) : “Alpha-amylase of Escherichia coli, mapping and cloning of the structural gene, maIS, and identification of its product as a periplasmic protein”; J. Biol. Chem. 261 (6), pp. 2946-2953), they showed no significant release of amylase under the experimental conditions. The (Gram-negative) E. coli strains JM 109, DH 10B and DH 12S also proved to be suitable hosts with respect to recognizing the ribosomal binding sites (Shine Dalgarno sequences) of the amylase genes of the (Gram-positive) Actinomycetales and recognizing the amino-terminal signal sequences and exporting out of the cell to produce the mature enzyme. Obviously, expression of amylases of streptomyces was also nontoxic to the host cells.

Purified genomic DNA from the strains to be studied, from Actinomycetales isolates in example 4, must therefore thus be partially cleaved and a manageable size range related to the expected size of the gene be ligated into appropriately linearized plasmid vectors. The minimum number of clones to be generated for each gene bank is based on the average insert size and the desired covering of the genome. In the case of the Actinomycetales α-amylases, the resulting insert size was from 3 to 5 kb, with a number of clones to be obtained of 40 000.

mAs an alternative to generating a genomic bank, it is also possible to construct a cDNA bank which is based on the mRNAs, i.e. the actually expressed genes. However, genomic banks are more likely to produce positive results in the case of genes whose expression in the starting organisms is only low or weak, however.

The clones obtained are assayed for recombination taking place, for example via the known blue/white selection, and finally for expression of the protein of interest. In the present example, the α-amylase was detected via its enzymic activity. Alternatively, however, an immunochemical detection or a screening via hybridization with known probes would also be possible, for example.

The latter possibility is particularly suitable for genes which are expressed in the host cells weakly, if at all. To this end, it is possible to use, for example, nucleic acid fragments as can be obtained by the PCR described in examples 1 to 3. Those probes which can be derived on the basis of the partial sequences or primer sequences indicated in the sequence listing may also be suitable. When translating the amino acid sequence back to a nucleotide sequence, the particular codon usage should be taken into account.

Said codon usage depends on the starting organisms from which the gene bank has been generated.

It is then possible to derive the corresponding DNA sequences from clones tested positively, according to methods known per se. Moreover, said clones are the starting points for all subsequent clonings, modifications, etc.

In the case of the Streptomyces sp. B327* Actinomycetales isolate (see example 2), the sequence listing depicts the complete DNA sequences obtained in this way and amino acid sequences derived therefrom under SEQ ID NO. 5 and 6.

The open reading frame of Streptomyces sp. B327* α-amylase (SEQ ID NO. 5) starts with a GTG start codon which is translated into methionine rather than valine to initiate translation. This is also indicated in the sequence listing in the section “misc_feature”: for positions 1 to 3, the feature “INIT_MET” applies. In addition, sequences corresponding to the ribosomal binding sites, i.e. the Shine-Dalgarno sequences, are located within up to 10 nucleotides upstream of said start codon.

Overall, the gene comprises 1386 nucleotides coding for a total of 461 amino acids. The first 30 amino acids of these are predicted to be typical signal sequences of Gram-positive bacteria, as can be expected of a secreted enzyme. This is indicated in the sequence listing (SEQ ID NO. 5) by the feature “mat_peptide38 which applies to positions 91 to 1383, since this region codes for the mature protein. In the amino acid sequence (SEQ ID NO. 6), positions 201 to 300 correspond to the region which was amplified in PCR typing (example 3), using the primer pair GEX024/GEX026.

The DNA sequences and amino acid sequences obtained for the native and the mature protein of Streptomyces sp. B327* α-amylase and the corresponding nucleotide sequence were used to screen the following databases for the most similar entries: Genpept/GenBank (National Center for Biotechnology Information NCBI, National Institutes of Health, Bethesda, Md., USA) and Swiss-Prot (Geneva Bioinformatics (GeneBio) S.A., Geneva, Switzerland; http://www/genebio.com/sprot.html). The search was carried out on different days, in each case via the server of the EMBL-European Bioinformatics Institute (EBI) in Cambridge, United Kingdom (http://www.ebi.ac.uk). The sequence comparisons were carried out according to the FASTA method (W. R. Pearson, D. J. Lipman, PNAS (1988) 85, pp. 2444-2448), i.e. using the Fasta3 program (Template: blosaum62 and default parameters). The result is listed in table 2.

TABLE 2
Result of the search in the Genpept/GenBank and Swiss-Prot
databases via the EMBL server, using the amino acid sequences
of the native (complete) and the mature protein (without
leader peptide) of Streptomyces sp. B327* α-amylase
and the corresponding DNA sequences, in each case carried
out using the FASTA program.

| | | | Homology | Closest |
|---|---|---|---|---|
| | | | in % | hit |
| Sequence | Database | Date | identity | Acc. No. |
| complete DNA | EMBL 1.0, 2000; | 01.18.01 | 83.5 | U51129 |
| | 65.0, 2000; | | | |
| | GenBank 121, | | | |
| | 2000, 1.0 2001 | | | |
| DNA without | EMBL 1.0, 2000; | 01.16.01 | 84.8 | U51129 |
| Leader | 65.0, 2000; | | | |
| | GenBank 121, | | | |
| | 2000, 1.0 2001 | | | |
| complete | Genpept/GenBank | 01.24.01 | 80 | U51129 |
| protein | 121, 2000 | | | |
| complete | Swiss-Prot | 01.04.01 | 78.7 | P27350 |
| protein | 39.0, 2000, | | | |
| | EMBL 1.0, 2000 | | | |
| protein | Genpept/GenBank | 01.24.01 | 82.4 | U51129 |
| without | 121, 2000 | | | |
| leader | | | | |
| protein | Swiss-Prot | 01.16.01 | 81.2 | P27350 |
| without | 39.0, 2000, | | | |
| leader | EMBL 1.0, 2000 | | | |

At the protein level, the Streptomyces sp. B327* α-amylase is most homologous to the GenBank database entry U51129; the homology is 80% identity across the sequence of the native protein and 82.4% for the mature protein. This most similar enzyme is Streptomyces albus α-amylase. In contrast, a search in the Swiss-Prot database produced values of 78.7 and 81.2%, respectively, identity for the entry P27350 as best hit which is Streptomyces thermoviolaceus α-amylase. FIG. 5 depicts an alignment of the amino acid sequences of the α-amylases of Streptomyces sp. B327* and Streptomyces albus, which sequences are referred to there as U51129, and B327*, respectively.

Thus, any amylolytic proteins whose amino acid sequence is at least 85% and, increasingly preferably, 87.5%, 90%, 92.5%, 95%, 96%, 97%, 98%, 99%, and very particularly 100%, identical to the amino acid sequence indicated in SEQ ID NO. 6 are claimed in the present application. This applies preferably to positions 31 to 461 of the mature protein, i.e. as far as this can be estimated from the known sequences.

Said degree of identity applies preferably in positions 31 to 461 and particularly preferably in positions 210 to 300 of the increasingly preferred embodiments of more than 95% identity, since the sequence found to be most similar to the latter amino acid region which corresponds to the consensus sequence of FIG. 3 and SEQ ID NO. 263 is, as already indicated in table 1, a database entry which is 92% identical to said region.

This entry is Y13332, i.e. Streptomyces sp. TO1 α-amylase.

The object of the invention is very particularly achieved by those amylolytic proteins whose amino acid sequence is identical to the amino acid sequence indicated in SEQ ID NO. 6, preferably in positions 31 to 461, particularly preferably in positions 210-300.

This protein found in Streptomyces sp. B327* may be expressed heterologously in various host cells, as illustrated in detail in examples 5 and 6. It is possible to use, for example, Streptomyces lividans TK24 which itself does not secrete any measurable amounts of amylase into liquid media. An example of a suitable expression vector is the expression vector pAX5a (Faβ S. H., Engels, J. W. 1996, “Influence of Specific Signal Peptide Mutations on the Expression and Secretion of the alpha-Amylase Inhibitor Tendamistat in Streptomyces lividans”, J. Biol. Chem., Volume 271 (Number 25), pp. 15244-15252; FIG. 6). Expression in this vector is under the control of the constitutive ermE promoter. Alternatively, expression in E. coli DH 12S, for example, is possible.

Example 6 investigates the enzymic properties of this heterologously produced protein. According to this, the maximum activity of Streptomyces sp. B327* α-amylase is at 41.3° C. Said amylase is most stable in the weakly acidic to neutral pH range, as determined via its activity. It is more active in a medium with low SDS content than in one without SDS. The activity is slightly reduced in the presence of chelators of divalent cations. These properties make Streptomyces sp. B327* α-amylase an interesting candidate for industrial applications. Further developments of this enzyme via molecular-biological methods are, within the similarity range defined above, incorporated within the scope of protection of the present application.

Commercial applications of this amylolytic protein are illustrated in more detail and by way of example further below.

An α-amylase of the invention was also obtained from the Actinomycetales isolate B400B, in the same way as described above for Streptomyces sp. B327* α-amylase. The complete DNA sequence of the former and the amino acid sequence derived therefrom are depicted in the sequence listing under SEQ ID NO. 7 and 8.

The open reading frame of the nucleotide sequence comprises 1377 nucleotides in total. It starts with a TTG start codon which, like in the corresponding enzyme in Streptomyces sp. B327*, is translated as methionine rather than leucine to initiate translation. This is also indicated in the sequence listing (SEQ ID NO. 7) in the section “misc_feature”: the feature “INIT_MET” applies to positions 1 to 3. The subsequent region likewise comprises the ribosomal binding sites.

The first 29 amino acids are predicted to be an export signal sequence (of the proenzyme) so that the mature protein begins with the amino acid sequence TPPGE. This is indicated in the sequence listing (SEQ ID NO. 7) by the feature “mat_peptide” which applies to positions 88 to 1374, since this region codes for the mature protein. SEQ ID NO. 8 depicts the complete amino acid sequence in which the mature protein starts at position 30. The positions 200 to 296 correspond to the region which was amplified in PCR typing (example 3) using the primer pair GEX024/GEX026. The protein comprises 458 amino acids.

This enzyme and, respectively, the DNA coding for this enzyme were subjected to the same database searches as the enzyme of strain B327*; table 3 summarizes the results of said searches.

TABLE 3
Result of the search in the Genpept/GenBank and Swiss-Prot
databases via the EMBL server, using the amino acid sequences
of the native (complete) and the mature protein (without
leader peptide) of Streptomyces sp. B400B α-amylase
and the corresponding DNA sequences, in each case carried
out using the FASTA program.

| | | | Homology | Closest |
|---|---|---|---|---|
| | | | in % | hit |
| Sequence | Database | Date | identity | Acc. No. |
| complete | EMBL 1.0, 2000; | 01.19.01 | 81.9 | U08602 |
| DNA | 65.0, 2000; | | | |
| | GenBank 121, | | | |
| | 2000, 1.0 2001 | | | |
| DNA | EMBL 1.0, 2000; | 01.17.01 | 82.6 | U08602 |
| without | 65.0, 2000; | | | |
| Leader | GenBank 121, | | | |
| | 2000, 1.0 2001 | | | |
| complete | Genpept/GenBank | 01.24.01 | 76.8 | U08602 |
| protein | 121, 2000 | | | |
| complete | Swiss-Prot | 01.04.01 | 71.6 | P08486 |
| protein | 39.0, 2000, | | | |
| | EMBL 1.0, 2000 | | | |
| protein | Genpept/GenBank | 01.24.01 | 77.7 | U08602 |
| without | 121, 2000 | | | |
| leader | | | | |
| protein | Swiss-Prot | 01.16.01 | 72.2 | P08486 |
| without | 39.0, 2000, | | | |
| leader | EMBL 1.0, 2000 | | | |

At the protein level, the Streptomyces sp. B400B α-amylase is most homologous to the GenBank database entry U08602; the homology is 76.8% identity across the sequence for the native protein and 77.7% for the mature protein. This most similar enzyme is Streptomyces sp. (GenBank Acc. No. U8602) α-amylase. In contrast, a search in the Swiss-Prot database produced values of 71.6 and 72.2%, respectively, identity for the database entry P08486 as best hit which is Streptomyces hygroscopicus α-amylase. FIG. 5 depicts an alignment of the amino acid sequences of the α-amylases of Streptomyces sp. B400B and Streptomyces sp. (GenBank Acc. No. U8602); the amino acid sequences in question are referred to there as U08602, and B400B, respectively.

Thus any amylolytic proteins whose amino acid sequence is at least 80% identical and, increasingly preferably, 82.5%, 85%, 87.5%, 90%, 92.5%, 95%, 96%, 97%, 98%, 99%, and very particularly 100%, identical to the amino acid sequence indicated in SEQ ID NO. 8 are claimed in the present application.

This applies preferably to positions 30 to 448 of the mature protein, i.e. as far as this can be estimated from the known sequences.

This applies particularly preferably to positions 200 to 296, since the sequence found to be most similar to this amino acid region which corresponds to the consensus sequence of FIG. 3 and SEQ ID NO. 263 is, as already indicated in table 1, a database entry (M15540) which is only 74.2% identical to said region and which is again the Streptomyces hygroscopicus α-amylase, since the EMBL entry M15540 corresponds to the entry P08468 in Swiss-Prot.

The object of the invention is very particularly achieved by those amylolytic proteins whose amino acid sequence is identical to the amino acid sequence indicated in SEQ ID NO. 8, preferably in positions 30 to 448, particularly preferably in positions 200 to 396.

This protein found in Streptomyces sp. B400B may be expressed heterologously in various host cells, for example Streptomyces lividans or E. coli, as illustrated in detail in examples 4 and 5 and already indicated further above for Streptomyces sp. B327* amylase. It is thus available for all commercial applications, as are illustrated in detail and by way of example further below.

Further embodiments of the present invention are fragments of amylolytic proteins or amylolytic proteins obtainable by deletion mutation, which can be derived from any of the above-described proteins.

This includes, for example, those fragments which contribute to the complexing of a substrate or to the formation of a structural element required for hydrolysis. They may be individual domains, for example. Such fragments may be less expensive to produce, may no longer possess particular, possibly disadvantageous characteristics of the starting molecule, such as possibly an activity-reducing regulatory mechanism, or may develop a more advantageous activity profile. Protein fragments of this kind may also be prepared non-biosynthetically, for example chemically. Methods of this kind are known, for example, from “Lexikon der Biochemie” [Encyclopedia of Biochemistry], Spektrum Akademischer Verlag, Berlin, 1999, volume 2, pp. 194-196. Chemical synthesis, for example, may be advantageous when chemical modifications are to be carried out after synthesis.

The generation of amylolytic deletion variants appears useful for deleting inhibiting regions, for example. The result may be, in addition to the deletions, both a specialization and an extension of the application range of the protein.

According to WO 99/57250, it is thus possible, for example, to provide a protein of the invention or parts thereof via peptidic or nonpeptidic linkers with binding domains of other proteins and thereby to render substrate hydrolysis more effective. It is likewise possible to link amylolytic proteins of the invention also to proteases, for example, in order to obtain bifunctional proteins.

The proteins and signal peptides obtainable by cleaving the N-terminal amino acids from preproteins may also be regarded as naturally produced fragments or deletion-mutated proteins. A cleavage mechanism of this kind may also be used in order to initially provide recombinant proteins with specific cleavage sites with the aid of particular sequence regions recognized by signal peptidases. Thus it is possible to activate and/or deactivate proteins of the invention in vitro. It is also possible to remove by deletion individual regions comprising in each case no more than 15 consecutive, and increasingly preferably no more than in each case 14, 13, 12, 11, 10, 9, 8, 7, 6, 5, 4, 3, 2 consecutive amino acids and one amino acid from the molecule. This is particularly expedient when the contribution to the washing performance is not reduced or is even improved thereby.

According to the statements made above, increasingly preference is given to those fragments or deletion variants which comprise portions which, within the homology values indicated above, correspond completely or partly to positions 210 to 300 of SEQ ID NO. 6 and, respectively, 200 to 296 of SEQ ID NO. 8, and particularly to those which comprise portions which, within the homology values indicated above, correspond to positions 8 to 93 of the consensus sequence of FIG. 3 and SEQ ID NO. 263.

The object of the invention is furthermore achieved by amylolytic proteins obtainable by insertion mutation or by amylolytic chimeric proteins, which comprise, at least in a portion imparting an amylolytic activity, any of the above-described proteins, particularly a corresponding mature protein, very particularly a portion of the above-described proteins which corresponds to positions 8 to 93 according to SEQ ID NO. 263.

According to the invention, the proteins obtained by the fusion are intended to have, cause or modify a function which is in the broadest sense amylolytic or a function supporting the hydrolysis of α-1,4-glycosidic bonds. That function may be exerted or modified by a molecule part which is derived from a protein of the invention and which is within the similarity range claimed above for this molecule part.

Such molecule parts may be portions which may be recognized via their consensus sequences or boxes as being homologous to the enzymes from other organisms. Such regions usually impart to the enzyme its characteristic enzymic functions and may also be distributed across various domains, i.e. globular regions of the protein molecule. The invention therefore also relates to those chimeric proteins which, owing to their construction, have, where appropriate, a lower identity across their entire amino acid and/or nucleotide sequence than defined above for the similarity range of the invention but which may be assigned to said range in at least one of the regions introduced by fusion and which exert in this portion the same functions as in an amylase which is within the above-defined homology range.

This applies to the proteins which can be derived from SEQ ID NO. 6 or 8 but also to the regions outside the consensus sequence depicted in SEQ ID NO. 263, since said sequence defines especially a highly variable region of α-amylases, as shown in example 1.

Owing to their similarity in principle, the same also applies to variants to be obtained by insertion mutation of the abovementioned amylolytic proteins. The purpose of insertion mutagenesis is, in particular, to combine individual properties of proteins of the invention with those of other proteins. Proteins or chimeric proteins according to the invention are obtainable by insertion mutation if the regions which can be traced back via their homology to the abovementioned sequences have appropriate homology values and the variant obtained has an amylolytic function in the broadest sense due to said regions.

Thus it is possible, for example, by applying the teaching of WO 99/57250 to couple such an enzyme to a cellulose binding domain in order to increase the interaction with the substrate. Analogously, it is also possible, for example, to fuse other detersive or cleaning-active enzymes to an amylase of the invention. In this connection, it is in principle immaterial whether the fusion takes place at the N or C terminals or via insertion.

Amylolytic derivatives of the abovementioned proteins mean those proteins which derive via chemical or biological, in particular molecular biological, modifications from those proteins which themselves have amylolytic activity or which support the hydrolysis of internal α-1,4-glycosidic bonds and which are within the similarity range indicated above. Increasing preference is given to derivatives of the correspondingly preferred starting molecules.

These molecules are in particular molecules which can be obtained from low molecular weight compounds or from polymers by chemical coupling. The purpose of such a modification, for example according to the teaching of WO 00/22103, may be a reduction in allergenic action, an optimization of the enzymic parameters, according to WO 99/58651, or an increase in stability, according to EP 1088887. The proteins may also be modified via glycosylation by -applying the teaching of WO 00/26354, for example.

Depending on the obtainment, working-up or preparation of a protein, such a protein may be associated with various other substances, in particular if it has been obtained from natural producers of said protein. It may then, but also independently thereof, have been specifically admixed with particular other substances, for example to increase its storage stability. Derivatives therefore also means any preparations of the abovementioned proteins. This is also independent of whether or not they actually produce said enzymic activity in a particular preparation, since it may be desired that they have only low activity, if any, during storage and produce said activity only when used. This may be controlled, for example, via the folding state of the protein or may result from the reversible binding of one or more accompanying substances or from another control mechanism.

The proteins of the invention may, especially during storage, be protected by stabilizers from, for example, denaturation, decay or inactivation, for example due to physical influences, oxidation or proteolysis. Combinations of stabilizers which complement or enhance one another are also frequently used.

One group of stabilizers are reversible protease inhibitors such as, for example, benzamidine hydrochloride and leupeptin, borax, boric acids, boronic acids, their salts or esters, peptide aldehydes or purely peptidic inhibitors such as ovomucoid or specific subtilisin inhibitors. Further familiar enzyme stabilizers are amino alcohols such as mono-, di-, triethanol- and -propanolamine, aliphatic carboxylic acids up to C₁₂, dicarboxylic acids, lower aliphatic alcohols, but especially polyols such as, for example, glycerol, ethylene glycol, propylene glycol or sorbitol. Calcium salts are also used, such as, for example, calcium acetate or calcium formate, magnesium salts, a very large variety of polymers such as, for example, lignin, cellulose ethers, polyamides or water-soluble vinyl copolymers, in order to stabilize the enzyme preparation especially against physical influences or pH fluctuations. Reducing agents and antioxidants such as, for example, sodium sulfite or reducing sugars increase the stability of the proteins against oxidative decay.

The object of the invention is also achieved by amylolytic proteins or derivatives which share at least one antigenic determinant with one of the abovementioned proteins or derivatives.

For not only the pure amino acid sequence of a protein but also the secondary structural elements and three-dimensional folding thereof are crucial for exerting enzymic activities. Thus, domains whose primary structures differ distinctly from one another can form structures which substantially correspond spatially and thus make identical enzymic behavior possible. Such common features in the secondary structure are usually recognized as corresponding antigenic determinants by antisera or pure or monoclonal antibodies. Immuno-chemical crossreactions thus make it possible to detect and classify proteins or derivatives which are structurally similar to one another. Secondly, the immunological crossreaction can readily detect that an amylase of the invention is actually active in an appropriate agent, for example a detergent or cleaning agent.

Therefore, the scope of protection of the present invention especially also includes those proteins or derivatives which have amylolytic activity and which can be assigned to the above-defined proteins or derivatives of the invention, albeit possibly not via their homologies in the primary structure, but nevertheless via their immunochemical relationship.

Conversely, proteins or derivatives of the invention may be used for obtaining, identifying or studying related amylase genes or amylases. This is possible, for example, via any molecular-biological methods which use antibodies against appropriate regions, for example when screening an expression gene bank.

The object of the invention is preferably achieved by the above-defined amylolytic proteins or derivatives if they are obtainable from natural sources. These include samples of natural habitats, scaling-up cultures, cell cultures, isolates or cultured single organisms. Preference is given to microorganisms, since they can be cultured according to the methods established in the prior art and be used directly for obtaining proteins or as starting points for molecular-biological methods.

However, they may also be organisms which themselves can be cultured only with difficulty, if at all, but whose proteins or whose DNA can be isolated from natural habitats and analyzed via methods known per se. Mixtures of such organisms are also possible. Preference is given in each case to those strains which produce the amylolytic protein under controllable conditions and release said protein into the surrounding medium.

Preferred microorganisms are, not least due to their protein synthesis mechanism and export mechanism, Gram-positive bacteria of which preference is given to those of the order Actinomycetales, since there are appropriate methods available therefor, not least owing to the information in the examples of the present application. As mentioned in the examples, the sequences for amylolytic proteins, on which the present application is based, in particular for part of the consensus sequence of SEQ ID NO. 263, were determined from Actinomycetales.

Further preference is given to amylolytic proteins or derivatives which are characterized in that they are obtainable from a Streptomyces species, since in a collection of microbial isolates of the order Actinomycetales, with the majority being of the genus Streptomyces, for example, more than 200 α-amylases and their natural producer strains were found and made available by the present application. Among these, preference is given to the isolates or species Streptomyces sp. B327* and Streptomyces sp. B400B which have been deposited under accession numbers DSM 13990 and DSM 13991 with the Deutsche Sammlung von Mikroorganismen und Zellkulturen GmbH (DSMZ), since from these the two particularly preferred embodiments of proteins of the invention were obtained, whose DNA and amino acid sequences are indicated in the sequence listing under SEQ ID NO. 5 to 8.

Some Actinomycetales can produce more than one α-amylase. In this case, it may be advantageous to culture a natural producer of a plurality of α-amylases which can possibly complement one another with respect to their biochemical properties. This may be utilized, for example, for producing an enzyme cocktail with a broad action range. Strains of this kind thus characterize particularly advantageous embodiments of the present invention.

SEQ ID NO. 34 to SEQ ID NO. 262 describe the amino acid sequences which are derived from particular PCR products, as illustrated above and in example 1. They have been obtained by reacting the nucleic acids isolated from a collection of several hundred Actinomycetales isolates as templates in polymerase chain reactions with the primer pairs GEX024 (SEQ ID NO. 9)/GEX026 (SEQ ID NO. 10) and GEX029 (SEQ ID NO. 11)/GEX031 (SEQ ID NO. 12). The positions 8 to 93 according to the consensus sequence of SEQ ID NO. 263 are located within the primers GEX024 and GEX026 so that they can be regarded as particularly characteristic of the sequences found. Thus the nucleic acids coding for these regions also characterize correspondingly preferred embodiments. The consensus sequence depicted in FIG. 3 and SEQ ID NO. 263 was derived from these individual sequences and defines a sequence space. Thus the present invention provides amylases which comprise a portion which can be described substantially by said consensus sequence.

Therefore, the corresponding nucleic acids must also be regarded as solutions to the object of the invention and thus as separate subject matter of the invention for the following reasons: firstly, proteins become molecular-biologically accessible when the corresponding genes are available. This applies to identification and characterization via mutagenesis up to biotechnological production. The nucleotide sequences derived due to the codon usage which varies between the different organisms and ribonucleic acids are also included, since functional enzymes can be derived from them, too. Corresponding nucleic acids may also be used for obtaining, identifying or studying amylase genes. Thus it is possible, for example, to design corresponding probes for screening gene banks.

Secondly, the abovementioned method for finding the α-amylases in question, which is detailed in the examples, is based on the PCR and thus on the corresponding nucleic acids. The sequence listing indicates not least the nucleotide sequences of two particularly preferred α-amylases (SEQ ID NO. 1, 3, 5 and 7); the nucleic acids coding for the remaining sequences can be deduced according to methods known per se.

Thus nucleic acids are claimed which code for amylolytic proteins whose amino acid sequences comprise a portion of which 98% and, increasingly preferably, 98.25%, 98.5%, 98.75%, 99%, 99.25%, 99.5%, 99.75%, and particularly preferably 100%, is described by the consensus sequence of SEQ ID NO. 263, in particular via the subregion corresponding to positions 8 to 93.

Nucleic acids which code for the variants possible according to the consensus sequence may be prepared according to generally known methods. For example, the “Lexikon der Biochemie” [Encyclopedia of Biochemistry], Spektrum Akademischer Verlag, Berlin, 1999, introduces in volume 1, pp. 267-271 methods for de-novo synthesis of DNA and in volume 2, pp. 227-229, the polymerase chain reaction (PCR). The sequences may be predefined according to the generally known coding system for amino acids, where appropriate using a codon usage which is characteristic of particular genera. Moreover, the DNA sequences indicated in SEQ ID NO. 1 and 3 may be used as starting points for synthesizing further nucleotide sequences by introducing the corresponding point mutations in those positions of said sequences which correspond to the desired variations of the invention.

For this purpose, all common and convenient methods may be used, as are known, for example, from the manual

Fritsch, Sambrook and Maniatis “Molecular cloning: a laboratory manual”, Cold Spring Harbor Laboratory Press, New York, 1989. As example 5 indicates, a PCR may also be used for introducing individual base substitutions into DNA.

An alternative possibility is to find, via a PCR, as described further above and in the examples, on nucleic acids from isolates of natural sources or of known strains, new genes or gene fragments whose derived amino acid sequence is described by the consensus sequence of SEQ ID NO. 263.

As the examples illustrate on the basis of the α-amylases from B327* and B400B, the complete genes belonging to the partial sequences may be found, for example, by expression cloning, screening of gene banks or comparable method steps. These include, for example, the cloning of genomic or cDNA into an expression vector with an α-amylase deletion mutant. Streptomyces or Bacillus species are preferred for intermediate cloning steps.

The genetic and protein-biochemical methods listed under the term protein engineering in the prior art are also based on the nucleotide sequence. Such methods may be used to further optimize proteins of the invention with regard to various uses, for example by point mutagenesis or fusion with sequences of other genes.

The object of the invention is preferably achieved by nucleic acids which code for amylolytic proteins whose amino acid sequences comprise a portion which is 98% and, increasingly preferably, 98.25%, 98.5%, 98.75%, 99%, 99.25%, 99.5%, 99.75%, and particularly preferably 100%, identical to any of the amino acid sequences indicated in SEQ ID NO. 34 to SEQ ID NO. 262, in particular via the subregion corresponding to positions 8 to 93 according to the consensus sequence of SEQ ID NO. 263.

For the definition of the abovementioned sequence space was based on said α-amylases which, on the other hand, embody proteins or protein fragments which have been successfully assayed for amylase activity. Moreover, as summarized in table 1, there are up to now no known proteins which are more than 97% identical to any of these protein fragments.

The nucleotide sequence to be deduced for each of these fragments may be readily introduced in mutagenesis, in particular optimization, steps or used for screening for the complete sequences. This is illustrated in the examples on the basis of the α-amylases of B327* and B400B. A corresponding positive result can be expected for any other of the fragments disclosed in the sequence listing and SEQ ID NO. 13 to SEQ ID NO. 242. A single negative result which may occur here may be attributed to strain-specific characteristics. Thus it is conceivable, for example, that the gene involved is a gene which has been produced by duplication and then been inactivated by mutation or a gene which is not activated in the living cells for previously unknown reasons.

Thus each nucleic acid coding for the fragments disclosed in SEQ ID NO. 13 to SEQ ID NO. 242 is also an alternative solution to the object on which the invention is based. At least it maps the path to a complete α-amylase and characterizes the latter in the region corresponding to the consensus sequence of SEQ ID NO. 263.

Further preferred embodiments of this subject matter of the invention are nucleic acids coding for amylolytic proteins whose amino acid sequence comprises a portion which is 98% and, increasingly preferably, 98.25%, 98.5%, 98.75%, 99%, 99.25%, 99.5%, 99.75%, and particularly preferably 100%, identical to any of the amino acid sequences indicated in SEQ ID NO. 45, 83, 97, 98, 101, 108, 109, 111, 112, 113, 115, 116, 232, 234, 236, 238, 239 and 241 to any of said sequences or which can be traced back in each homologous position directly to any of said sequences, in particular via the subregion corresponding to positions 8 to 93 according to the consensus sequence of SEQ ID NO. 263.

For in example 1, the PCR products of DNA to be obtained using the two primer pairs GEX024/GEX026 and GEX029/GEX031 were obtained from the following isolates or strains of Streptomyces sp.: B101A, B114C, B134, B135A, B138A, B152A, B153(B), B156B, B157C, B158A, B160B, B161A, B373, B375, B380, B390, B392A and B394. FIG. 2 depicts the gel-electrophoretic fractionation thereof. Sequencing thereof and derivation of the corresponding amino acid sequence resulted in the amino acid sequences mentioned.

Thus any nucleic acids which code for proteins having a portion which are within the above-defined similarity range are embodiments of this subject matter of the invention. As illustrated above, this applies in particular to the subregion corresponding to positions 8 to 93 according to the consensus sequence of SEQ ID NO. 263.

Further preferred embodiments of this subject matter of the invention are nucleic acids coding for amylolytic proteins whose amino acid sequence comprises a portion which is 95% and, increasingly preferably, 96%, 96.5%, 97%, 97.5%, 98%, 98.5%, 99%, 99.5% and particularly preferably 100%, identical to any of the amino acid sequences indicated in SEQ ID NO. 2, 4 and 208, in particular via the subregion corresponding to positions 8 to 93 according to the consensus sequence of SEQ ID NO. 263, very particularly preferably a nucleic acid having a nucleotide sequence indicated in SEQ ID NO. 1 or 3.

For the complete proteins obtained via said partial sequences were tested for their biochemical properties in example 6. They therefore seem to be promising candidates for use in industrial processes or for further optimization with regard to industrial processes. Similar properties are to be expected for the proteins which may be obtained starting from the nucleic acids of this subject matter of the invention.

Particularly preferred embodiments are those nucleic acids which code for an amylolytic protein whose amino acid sequence can be traced back via a conserved amino acid substitution, preferably directly, in each individual homologous position to any of the two sequences SEQ ID NO. 6 or SEQ ID NO. 8.

For, as has already been illustrated above, these two amino acid sequences define a sequence space which encompasses the further embodiments of the present invention via conserved substitutions or direct adoption of the amino acids prescribed in said two sequences. Accordingly, the corresponding nucleic acids are also embodiments of the present invention.

Referring back to the amino acid sequence is necessary, because direct adoption of one or other nucleobase from the two prescribed nucleotide sequences might result in a difference in the codons and thus in non-conserved substitutions at the amino acid level. The scope of protection includes only those nucleotide sequences which code for proteins belonging to the same sequence space. They are obtained by adopting the relevant codons or by substituting with those codons which code for the same amino acids (synonymous codons) or conserved amino acids according to the groups indicated above.

Preferred embodiments of this subject matter of the invention are those nucleic acids which are sufficiently similar to the α-amylase gene of Streptomyces sp. B327*, since they can be expected to code for enzymes equally as promising as the α-amylase characterized in example 6 or to be introduced in methods for the optimization thereof.

Table 2 reveals that the gene most similar to B327* at the DNA level is the α-amylase gene from Streptomyces albus, filed in GenBank under accession number U51129. Said gene is 83.5% identical with respect to the complete sequence indicated in SEQ ID NO. 5 and 84.8% identical with respect to the portion coding for the mature protein.

This portion is encoded by nucleotides 91 to 1383 in the sequence indicated in SEQ ID NO. 5. The nucleotides 628 to 900 correspond to the amino acids of the consensus sequence (FIG. 3 and SEQ ID NO. 263).

Thus those nucleic acids coding for amylolytic proteins, whose sequence is at least 85% identical to the nucleotide sequence indicated in SEQ ID NO. 5, preferably in positions 91 to 1383, particularly preferably in positions 628 to 900, are claimed as preferred representatives of this subject matter of the invention.

Increasingly preferably those nucleic acids coding for amylolytic proteins are claimed whose sequence is at least 87.5%, 90%, 92.5%, 95%, 96%, 97%, 98%, 99% and 100% identical to the nucleotide sequence indicated in SEQ ID NO. 5, preferably in positions 91 to 1383, particularly preferably in positions 628 to 900.

Preferred embodiments of this subject matter of the invention are those nucleic acids which are sufficiently similar to the α-amylase gene of Streptomyces sp. B400B, since they can be expected to code for enzymes equally as promising as the α-amylase characterized in example 6 or to be introduced in methods for the optimization thereof.

Table 3 reveals that the most similar gene at the DNA level is that for Streptomyces sp. α-amylase (GenBank Acc. No. U08602). These two sequences are 81.9% identical across the sequence coding for the native protein and 82.6% identical with respect to the sequence for the mature protein.

The mature portion is encoded by nucleotides 88 to 1374 of the sequence indicated in SEQ ID NO. 7. The nucleotides 598 to 888 correspond to the amino acids of the consensus sequence (FIG. 3 and SEQ ID NO. 263).

Thus those nucleic acids coding for amylolytic proteins, whose sequence is at least 85% identical to the nucleotide sequence indicated in SEQ ID NO. 7, preferably in positions 88 to 1374, particularly preferably in positions 598 to 888, are claimed as preferred representatives of this subject matter of the invention.

Increasingly preferably those nucleic acids coding for amylolytic proteins are claimed whose sequence is at least 85%, 87.5%, 90%, 91%, 92%, 93%, 94%, 95%, 96%, 97%, 98%, 99% and 100% identical to the nucleotide sequence indicated in SEQ ID NO. 7, preferably in positions 88 to 1374, particularly preferably in positions 598 to 888.

Correspondingly preferred embodiments of this subject matter of the invention are the nucleic acids which code for any of the above-defined amylolytic proteins or fragments.

This also includes those variants which in individual regions, although not over the entire length of their sequence, are within the above-defined similarity range. These include, for example, the nucleotide sequences which, as set forth above, have been obtained by insertion or deletion mutation, chimeric proteins or protein fragments. However, “antisense constructs” are, for example via individual subsections, also embodiments of the present invention, since they can produce sequence information about amylolytic proteins and be used for regulating the amylolytic activity. The mutants obtainable via molecular-biological methods known per se include in particular those having single specific base substitutions or randomized point mutations, deletions of individual bases or of partial sequences, fusions with other genes or gene fragments or other enzymes, insertions, shuffling mutagenesis or inversions. Mutations or modifications of this kind may represent particular embodiments for specific applications.

A mutagenesis of this kind may be carried out target-specifically or via random-type methods. This may be combined, for example, with a subsequent method for screening and/or selecting the cloned genes for activity. The genes obtained by mutation are subject to the scope of protection of the invention described herein, if they code for amylolytic proteins in the broadest sense and are within the above-defined similarity range; in particular, with regard to the latter, in homologous and functionally relevant regions.

As is obvious from the previous statements and from example 1, the methods indicated may be used to characterize numerous organisms in that the latter produce amylolytic proteins which comprise portions described by the consensus sequence of FIG. 3 or SEQ ID NO. 263.

Thus any natural organisms which comprise nucleic acids coding for any of the proteins or protein fragments defined in the sequence listing under SEQ ID NO. 263 are a separate subject matter of the invention. They may be isolated from natural habitats and cultured according to methods known per se, in particular those provided by the present application. In addition to any microbiological characterization, they can be identified by the fact that amplification products whose derived amino acid sequence can be described by the consensus sequence of FIG. 3 or SEQ ID NO. 263 can be obtained on the basis of their genomic DNA, using one of the PCR primers mentioned in example 1, in particular the pair GEX024/GEX026.

Preference is given to those natural organisms from which an amino acid sequence can be derived in this way, which is identical to any of the sequences indicated in the sequence listing under SEQ ID NO. 34 to 262 and particularly preferably to any of the sequences indicated under SEQ ID NO. 2, 4, 6 or 8. This applies to all organisms, for example eukaryotic cells, e.g. from cell cultures, or predominantly unicellular fungi such as yeasts.

This applies preferably to microorganisms, preferentially to bacteria, very particularly preferably to Gram-positive bacteria.

Among Gram-positive bacteria, preference is given to those of the order Actinomycetales, in particular of the genus Streptomyces.

Of these, in turn, preference is given to the two species Streptomyces sp. B327* and Streptomyces sp. B400B, in particular in the case of either of the two strains DSM 13990 or DSM 13991.

As illustrated above and in the examples, the multiplicity of α-amylases of the invention were provided via a specially developed method which therefore constitutes a separate subject matter of the invention.

Thus PCR-based methods for identifying and/or obtaining new amylases from a collection of organisms or nucleic acids are claimed, which methods are characterized in that PCR primers are used which in each case have a variable 3′ region and a 5′ region highly homologous to regions of known amylases.

The polymerase chain reaction (PCR) method is established in the prior art and is described, for example, in “Lexikon der Biochemie” [Encyclopedia of Biochemistry], Spektrum Akademischer Verlag, Berlin, 1999, volume 2, pp. 227-229. It is based on melting open, in the first step, a double-stranded DNA template at an elevated temperature, (2.) short single-stranded DNA molecules, the primers or primer oligonucleotides, binding to positions of the melted-open DNA, which are sufficiently homologous for hybridization, at low temperature, and (3.) extending said short DNA molecules from the 5′ to the 340 end at medium temperature as in natural DNA synthesis. This produces in the first cycle two copies of the DNA region which is located between the two primer binding sites in the initially melted-open DNA. In (4.) a cyclic reaction process, as is possible by using a heat-stable DNA polymerase, for example, an increasing number of cycles produces an exponentially increasing number of identical DNA molecules across the DNA region flanked by the two primer sequences.

The method of the invention is characterized by novel primers for the PCR, which are characterized by a variable 3′ section and a 5′ region which is highly homologous to DNA and/or amino acid regions of known amylases. The 5′ regions of the primers are preferably regions which were found to be conserved regions in a sequence comparison at the protein level, preferably at the DNA level, with various homologizable amylases and which are located adjacent to variable regions of the homologized genes or proteins in question. An assignment of this kind is depicted in FIG. 1, for example.

The border between the 3′ and 5′ primer regions defined in this way is identical to the border between the conserved and variable amylase regions, found via homologization. If doubt exists, the variable region starts where the synthesis (see below) has generated a variance.

High homology in this connection means that said primers, in their 5′ region, (a) are 100% identical to the regions under consideration of known enzymes or consensus sequences or (b) if they are less than 100% identical, still have a sufficient degree of matches in individual positions in order to hybridize with a likewise homologizable template DNA in the region suggested by sequence comparison in the hybridization phase of a PCR cycle and to make possible a DNA synthesis reaction in the further course of the PCR. Variable or degenerated PCR primers have at defined positions in each case a random mixture of a selection of two, three or four different nucleotides rather than one particular nucleotide, so that the identity of the primers rather than their overall length is varied. The degree of variability of the primer pool obtained increases exponentially with the number of variable individual positions. These variations may be introduced during synthesis of the primers, in particular chemical synthesis, according to methods known per se. This requires using, at the point in time at which the nucleotide for a particular position is incorporated, said reagent not as pure nucleotide but as nucleotide mixture. In this way, a number of primers with identical 5′ regions are produced which divide into numerous subpopulations containing any statistically possible nucleotides in the particular variable regions.

Said novel primers produce in the presence of gene sequences of amylolytic proteins a product band corresponding to said proteins. They render the variable region located in between accessible and are therefore also referred to as sequence anchors.

The template used for the PCR of the invention may be any DNA preparations. Owing to the comparatively small amount of work required for a single sample, it is possible to set up a plurality of reaction mixtures in parallel. The method of the invention is thus particularly suitable for screening a collection of nucleic acids prepared from a multiplicity of organisms according to methods known per se. The templates may also be cDNA or even mRNA preparations, if the PCR is designed accordingly, as is sufficiently described in the prior art.

In a variation of the reaction process, the degenerated primers are added only to the first reaction cycles and/or, after the first cycles, the primers which correspond to the constant regions of the primers important to the invention are added. A third possibility is to carry out two separate PCRs. In this way, the relevant genetic elements of the template are concentrated in the first cycles using the degenerated primers and then specifically amplified in the subsequent cycles.

The selection of said possibilities or further modifications, in particular in conducting the PCR, depends, for example, on the selectivity of primer binding or on the frequency of the DNA used as template containing the genetic element amplifiable in this way. The selectivity of the PCR can can be regulated via the reaction conditions in a manner known per se. At low temperatures, for example, the primers hybridize with DNA regions which are not identical and/or have only low homology; at higher temperatures, the selectivity increases. In this way it is possible to access α-amylase sequences which do not completely correspond to the previously known amylase genes, even in the highly homologous regions. On the other hand, primer binding must not be so unspecific that the proportion of genes not coding for amylases does not gain the upper hand compared to the desired product. The optimum must be determined in the individual case by varying the reaction conditions.

The multiplicity of possible variations make it possible to adapt to many different problems, for example to different starting DNA collections, and render the method of the invention a very flexible instrument for finding new amylase sequences.

This optimization is carried out against the background that, when studying DNA sequences from collections of related nucleic acids, advantageously a number of α-amylase sequences should be obtained which, although themselves diverse, do not fall short of a certain degree of homology to one another (see above), since they may then be used for defining a sequence space and for random mutagenesis methods such as shuffling, for example.

In this way, the primer combinations GEX029/GEX031 and in particular GEX024/GEX026 were found to be optimal for a collection of DNA from Actinomycetales in example 1.

The PCR products obtained are used for the search for complete amylase sequences. Said PCR is thus a screening step via which particular genes or gene fragments from a gene bank or collection of isolated nucleic acids are identified. The PCR fragments obtained or corresponding genes, including all of their deviations in individual positions, constitute a sequence space. They are also typed in this way, since they may be regarded via this common sequence as representatives of a particular type of enzyme.

This screening reduces the global diversity to the sequence space of those genes which code for particular enzymes having amylolytic activity. Said sequence space is of particular interest if it contains as large a sequence variability as possible of genes for such enzymes.

A preferred aim, to be taken into account even when constructing the primers, may be to obtain a variance space for particular enzyme regions such as particular enzymically interesting domains, for example. These are usually domains which provide particular partial activities such as, for example, structural elements or enzymically active amino acids. The fragments forming such a sequence space may be fused to other known enzymes or enzyme fragments. They may also be introduced into evolutive techniques for developing new enzymes. Such evolutive techniques for random recombination of various fragments are illustrated, for example, in the patent applications WO 98/05764, WO 97/35966, EP 590689 and EP 229046. A PCR-based method of this kind is, for example, the StEP method described in WO 98/42832.

Another possible use of the PCR products is the screening of gene banks which is carried out using the DNA fragments obtained by said PCR or parts thereof as probe. To this end, in particular hybridization methods by which sequences with high or low homology can be detected, depending on the stringency of the hybridizations, are established in the prior art. In this way it is possible to identify similar sequences for amylolytic proteins.

The PCR fragment and the sequence information obtained therewith itself may also be used as starting point for further PCR- and/or sequence-based methods for identifying, isolating or cloning the entire gene. To this end, for example, inverse PCR, A-PCR (anchored PCR) or primer walking are described in the prior art. The complete genes obtained in this way are finally tested for their starch-cleaving activity in an activity assay, for example after cloning into an expression vector.

All of these methods may be linked in a useful manner to a subsequent assay of the genes or proteins obtained via possible intermediate steps for amylolytic activity. These variations characterize preferred embodiments of this subject matter of the invention.

In one embodiment of this subject matter of the invention, the genes or gene fragments obtained are sequenced. Said sequencing may yield information about the identity of the fragments obtained, since, as mentioned, the method depends on the selectivity of the PCR conditions. Thus it is possible, with low-selectivity conditions, for PCR products produced due to unspecific primer binding to fake positive results. Optional sequencing of the products thus avoids unnecessary work. Large PCR products can be sequenced by applying primer walking, for example in a manner as carried out for the complete genes in example 4.

On the other hand, homologization, database searches and the definition of a sequence space may, as examples 1 to 3 show, already be carried out via the PCR products derived from a portion of the gene. The formation of a particular PCR product may characterize individual strains or larger groups such as species or genera and may represent a taxonomic classification feature. This is a valuable aid when culturing said strains.

One embodiment of methods of the invention is characterized in deriving from the genes or gene fragments obtained peptides which are assayed for an epitope of a known amylase via an immunochemical method.

Examples suitable for this are also the products of the PCR of the invention. Thus, for example, screening may be carried out with the aid of antibodies which have been synthesized according to methods known per se on the basis of the derived peptides. The amylolytic proteins and derivatives disclosed by the present application are available for particularly preferred embodiments.

As an alternative or in addition to sequencing or to an immunochemical method, α-amylases may also be identified owing to their enzymic activity.

Thus preference is given to those methods which are characterized in that peptides are derived from the genes or gene fragments obtained, which are assayed for a contribution to an amylolytic activity, preferably for amylolytic activity completely exerted by said peptides. To this end, the PCR product or the complete gene obtained via a gene bank screen may be expressed by cloning, for example as in example 5.

Particularly long PCR fragments, as result in particular from the choice of appropriate primers, may be cloned directly into expression vectors in the course of expression cloning, without providing accordingly other partial sequences in the vector. The clones obtained are then assayed for starch hydrolyzing activity, for example by plating out on starch-containing agar plates.

In a preferred embodiment, the 3′ region of at least one primer is highly variable or highly degenerated and/or the 5′ region of at least one primer is highly homologous to a region from known amylases.

Highly variable primers mean, for example, primers with 8-fold, 16-fold, 64-fold, 96-fold, 128-fold or even higher degeneracy or primers with intermediate variabilities. The primers are regarded as highly homologous when the constant 5′ regions deviate from the consensus sequence used for construction by no more than two nucleobases, preferably by no more than one nucleobase, with completely identical sequences being particularly preferred.

The primers derived on the basis of FIG. 1 are listed in example 1 in table 3 and in the sequence listing under SEQ ID NO. 9 to 33. The variable positions are indicated there in each case by the commonly used one letter codes stated, for example, in WIPO standard ST.25. Thus, for example, the primer GEX024 (SEQ ID NO. 9) has in two positions the abbreviations “s” and “r”, which may mean in each case either g or c (for s) and g or a (for r), respectively. Said primer thus has a 4-fold degeneracy. Correspondingly, it is understood that, for example, the degeneracy of the primer GEX026 (SEQ ID NO. 10) is likewise 4 fold, that of the primer GEX029 (SEQ ID NO. 11) is 96 fold and that of the primer GEX031 (SEQ ID NO. 12) is 8 fold.

A method of the invention is based on the fact that it is possible to identify regions which are conserved between a plurality of amylases and which may be utilized for constructing the constant regions of the primers of the invention, thereby becoming sequence anchors. Appropriate primers are listed in example 1; FIG. 1 depicts their position with respect to particular conserved regions.

Accordingly, preference is given to those methods of the invention which are characterized in that the 5′ region of at least one PCR primer is derived from a sequence region corresponding to a conserved amylase domain, preferably of a region corresponding to the amino acid positions (A) 58-91, (B) 94-141, (C) 155-207, (D) 295-345 or (E) 392-427 of the Streptomyces griseus α-amylase, particularly preferably corresponding to one of the domains β4 or β7 of the (αβ)₈ barrel structure.

The latter were successfully used in example 1 on Actinomycetales DNA.

The amino acid sequences provided by the present application and listed in the sequence listing may be used for synthesizing corresponding primers. This applies, for example, also to regions which are not located in the conserved regions but which emerge as common partial sequences in subpopulations, for example individual representatives of the consensus sequence of SEQ ID NO. 263.

Preference is thus given to methods which are characterized in that one, preferably two, PCR primers are used which can be derived from any of the amino acid sequences of a protein or fragment of the first subject matter of the invention.

On the other hand, preference is given to those methods which are characterized in that one, preferably two, PCR primers are used which result directly from any of the nucleotide sequences according to the second subject matter of the invention, since these already have a nucleotide sequence which should, owing to the codon usage, be suitable in particular for amplifying sequences from Actinomycetales or other Gram-positive bacteria.

Among these, preference is given to methods which are characterized in that at least one primer comprises any of the sequences indicated in the sequence listing under SEQ ID NO. 9 to 33, in particular any of the sequences indicated in the sequence listing under SEQ ID NO. 9 to 12.

Among these, preference is in turn given to methods which are characterized in that the primers are used in the combination of SEQ ID NO. 9 with SEQ ID NO. 10 or of SEQ ID NO. 11 with SEQ ID NO. 12, since these were, as example 1 shows, successfully used for obtaining a multiplicity of α-amylases.

Methods of the invention can be carried out on individual samples. On the other hand, however, preference is given to methods which are characterized in that the template DNA used is isolated nucleic acids of a plurality of samples which can be any biologically useful collections, including, for example, collections of organisms, cell cultures, isolates or cultured individual strains, or is isolated nucleic acids of a sum of organisms, cell cultures, isolates, cultured individual strains or isolated nucleic acids.

Isolated nucleic acid may be purified from samples, for example outdoor isolates, thereby avoiding an isolation step, for example. In this way it is possible to subject a very broad range of different biological sources to a screening of the invention.

As an alternative to obtaining outdoor isolates, it is also possible to obtain various strains from the generally accessible collections of strains or of genetically modified organisms, preferably microorganisms. It is also possible to study various strains from culture collections (DSMZ, ATCC, CBS) by said method.

The method of the invention may also be applied to cell cultures, as generated, for example, from eukaryotes, in particular from humans. This is useful, for example, when the enzymes to be obtained are to be used owing to a particular physiological action or if said enzymes are to be particularly well tolerated, the latter applying in particular to cosmetic applications.

Collections of this kind are preferably those of mainly unicellular microorganisms such as fungi, yeasts, bacteria or cyanobacteria. Preference is given to bacteria, since these are most readily accessible to the microbiological and genetic methods established in the prior art. Among these, particular preference is given to those of Gram-positive bacteria, since these come closest to industrial applications owing to their ability to export de novo synthesized proteins into the surrounding medium.

According to the remarks above and to the examples of the present application, increasing preference is given to the following methods:

Methods characterized in that the template DNA used is isolated nucleic acids of microorganisms, preferably of bacteria and particularly preferably of Gram-positive bacteria; those characterized in that members of the order Actinomycetales, in particular those of the genus Streptomyces, are involved and those characterized in that the members of the genus Streptomyces are Streptomyces sp. 327* or Streptomyces sp. B400B, in particular either of the two strains DSM 13990 and DSM 13991.

Preferred methods are characterized in that the genes or gene fragments obtained are introduced into an expression gene bank which is then assayed for amylolytic proteins or fragments of amylolytic proteins. Such an assay preferably involves hybridization of nucleic acids, an immunochemical method or an activity assay. Example 4 describes such an expression cloning.

The hybridization is carried out in a known manner, described, for example, in the manual by Fritsch, Sambrook and Maniatis “Molecular cloning: a laboratory manual”, Cold Spring Harbor Laboratory Press, New York, 1989, by using a nucleic acid of a known amylase as probe in order to identify the sequences substantially complementary thereto in the gene bank. The desired degree of homology may be controlled here via the choice of reaction conditions, i.e. the stringency. Identified sequences of the invention may be recognized via their homology to known amylases, with an activity assay serving as positive control.

Banks which already provide expression of the genes they contain are assayed on the basis of the gene products. Suitable for this are antibodies against known α-amylases, and the products found must likewise be checked for their amylolytic activity. These methods are also established in the prior art. Alternatively, the isolated cells of expression banks can be assayed directly for their activity by applying them to starch-containing agar plates, as indicated in the examples. Positive clones can then be recognized by their lysis halos.

Methods of one embodiment are characterized in that the isolated DNA is introduced into an expression vector which provides transcriptional and translational control elements and optionally one or more fragments of an amylase gene so that the overall result in a positive case is an amylase activity.

Suitable for this are in particular those vectors into which a fragment obtained is recombined in such a way that complete enzymes are obtained. This can be implemented by means of expression vectors which contain a gene deleted in such a way that cloning of the fragment cancels out exactly this deletion. This embodiment is particularly suitable for PCR fragments which are too short in order to code for a detectable amylolytic activity.

Particular preference is given to a method whose activity assay which follows the inventive PCR for screening isolated nucleic acids and which comprises three steps is as follows:

(a) screening a gene bank, preferably one with complete genes, with the aid of the PCR fragment obtained or a probe derived therefrom,

(b) expressing the genes or gene fragments identified in this way, and

(c) measuring the contribution to an amylolytic activity of the proteins or protein fragments obtained by said expression.

Said gene bank may conveniently be a genomic or a cDNA bank. It is possible to carry out the PCR of the invention and the activity assay on the same bank.

The term screening means a screening established in the prior art, in particular the abovementioned assaying of the banks via hybridization with nucleic acids. In this case, the partial sequences obtained by the PCR of the invention rather than partial sequences of known amylases are used. Screening the gene banks then serves to identify clones which comprise the corresponding complete amylase gene.

Alternative methods known from the prior art comprise using the PCR fragment obtained as starting point for an inverse PCR or an anchored PCR (a-PCR) or for sequencing by primer walking.

Preference is given to those methods in which the nucleic acids obtained after the PCR or the complete genes are introduced into cloning vectors and transformed into host cells, prior to the activity assay. They are thus more readily accessible to the abovementioned sequencing and/or further cloning steps, including cloning into expression vectors.

The host cells to be used for this purpose are preferably strains of the genera Escherichia coli, Streptomyces or Bacillus.

Methods of the invention are carried out not only with regard to individual sequences but also in order to obtain a multiplicity of sequences which are homologizable and via which a sequence space can be defined, which in turn refers to the particular amino acid sequences, since it is the aim of the method of the invention to identify correspondingly different proteins. Thus, the amino acid sequences derivable from the PCR products must be contemplated in each case.

Accordingly, preference is therefore given to methods which are carried out or repeated on so many different PCR templates that at least two different amino acid sequences having a common consensus sequence are obtained, in particular via individual domains, partial activities, structural elements or complete genes and/or proteins.

It is the aim of these studies to generate a sequence space whose individual representatives have such a ratio in homology and variance that said sequence space can be introduced to random methods of mutagenesis and enzyme development, for example shuffling mutagenesis.

Accordingly, preference is therefore given to methods which are characterized in that a consensus sequence with sequence identities of at least two sequences contained therein of at least 30%, preferably at least 40%, particularly preferably at least 50%, is obtained.

A separate subject matter of the invention is the possibility of using the proteins or nucleic acids provided by the present application for finding further α-amylases.

Thus the use of a protein or derivative according to the first subject matter of the invention for identifying an amylolytic protein is claimed, preferably in a method according to the fourth subject matter of the invention.

They may be used, for example, for deriving corresponding primers for methods of the invention. However, they may also be used for the abovementioned methods for generating antibodies for the immunochemical screening of an expression bank.

Nucleic acids according to the second subject matter of the invention may also be used for identifying and/or obtaining a new amylase, preferably in a method according to the fourth subject matter of the invention.

They may serve, for example, as template for developing primers for a PCR of the invention or may be available as deletion mutant in expression vectors so that cloning into said vector leads to complementation for expression.

A possible use for proteins obtained according to a method of the invention results from fusion or linkage to another protein, in particular for developing a new enzyme. In this way it is possible to prepare, for example, multifunctional enzymes by chemical coupling.

A possible use for nucleic acids obtained according to a method of the invention results from their use for fusion to another nucleic acid, in particular for developing a new enzyme. This may be carried out via specific fusion of particular sequences or via a random recombination method.

Uses of this kind for specific fusion, utilizing highly homologous regions of the amino acid sequences, common restriction cleavage sites of the nucleic acids or via PCR-based fusion, are preferred embodiments.

Another possibility is the use in a random recombination method, in particular by utilizing highly homologous regions of the amino acid sequences, common restriction cleavage sites of the nucleic acids or via PCR-based fusion.

The highly homologous regions of the amino acid sequences, which are usually conserved, function-carrying regions, may serve here as anchors for PCR primers, where appropriate of degenerated primers. Common restriction cleavage sites of the nucleic acids of two different genes make possible a direct fusion at the DNA level. Any methods established in the prior art and also the method of the invention, including its various embodiments, are available for combining the various gene regions.

Vectors comprising any of the nucleic acid regions of the second subject matter of the invention are included in a separate subject matter of the invention.

Such vectors are the preferred starting points for molecular-biological and biochemical studies of the gene in question or corresponding protein, for further developments of the invention and for amplifying and producing proteins of the invention.

Suitable vectors may be derived from bacterial plasmids, from viruses or from bacteriophages but may also contain elements of a wide variety of origins. Using the other genetic elements present in each case, vectors are able to establish themselves as stable units in the relevant host cells over several generations. In accordance with the invention, it is unimportant here whether they establish themselves as independent units extrachromosomally or integrate into a chromosome. The system of choice out of the numerous systems known in the prior art depends on the individual case. Decisive factors may be, for example, the copy number attainable, the selection systems available, among these especially resistances to antibiotics, or the culturability of the host cells capable of taking up said vectors.

Cloning vectors are preferred embodiments of this subject matter of the invention.

Said cloning vectors are, in addition to storage, biological amplification or selection of the gene of interest, suitable for characterization of the gene in question, for example via generation of a restriction map or sequencing. Cloning vectors are also preferred embodiments of the present invention, because they are a transportable and storable form of the claimed DNA. They are also preferred starting points for molecular-biological techniques not linked to cells, such as the polymerase chain reaction, for example.

Expression vectors are preferred embodiments of this subject matter of the invention.

Owing to the appropriate genetic elements, said expression vectors are capable of replicating in the host organisms optimized for the production of proteins and of expressing the contained gene there. Preferred embodiments are expression vectors which themselves carry all the genetic elements necessary for expression. Promoters regulate transcription of a gene. Examples of the former are natural promoters originally located upstream of the genes in question. Since in the case of most of the genes of the invention, however, said promoters cannot be assumed to be known, preference is given to those embodiments in which, after genetic fusion, known, other promoters are provided for regulation of the transgene. The promoter here may be a promoter of the host cell, a modified promoter or else a completely different promoter from a different organism. Preferred embodiments are those expression vectors which can be regulated by changing the culture conditions or by adding particular compounds, such as, for example, cell density or specific factors.

Expression vectors enable heterologous or homologous protein expression, depending on the particular genetic elements. Cell-free expression systems in which protein biosynthesis is mimicked in vitro may also be embodiments of the present invention. Such expression systems are likewise established in the prior art and are based on the genes provided in cloning or expression vectors.

This subject matter of the invention includes host cells which express or can be induced to express any of the proteins or derivatives of the invention, preferably by using an appropriate expression vector.

In-vivo synthesis of an amylolytic enzyme of the invention by living cells requires the transfer of the corresponding gene into a host cell, i.e. transformation thereof. Suitable host cells are in principle any organisms, i.e. prokaryotes, eukaryotes or cyanophytes. Preference is given to those host cells which are easily manageable genetically, with respect to, for example, transformation with the expression vector and its stable establishment. Moreover, preferred host cells are distinguished by good microbiological and biotechnological manageability. This relates, for example, to easy culturability, high growth rates, low requirements on fermentation media and good rates of production and secretion for foreign proteins. Frequently, it is necessary to determine experimentally the expression systems optimal for the individual case from the abundance of various systems available according to the prior art.

Preferred embodiments are those host cells whose protein production rate can be regulated, owing to genetic regulatory elements which may be provided, for example, on the expression vector or else may be present in said cells from the outset. Expression in said cells may be induced, for example, by controlled addition of chemical compounds used as activators, by changing the culturing conditions or on reaching a particular cell density. This makes possible a very economical production of the proteins of interest.

A variant of this experimental principle is expression systems in which additional genes, for example those provided on other vectors, influence the production of proteins of the invention. Said additional genes may be modifying gene products or those intended to be purified together with the protein of the invention, for example in order to influence its amylolytic function. They may be, for example, other proteins or enzymes, inhibitors or those elements which influence the interaction with various substrates.

In a preferred embodiment, the host cell is characterized in that it is a bacterium, in particular one which secretes the protein or derivative produced into the surrounding medium.

Preferred host cells are prokaryotic or bacterial cells. Bacteria usually distinguish themselves from eukaryotes by shorter generation times and lower demands on the culturing conditions. This makes it possible to establish cost-effective methods for obtaining proteins of the invention.

Host cells characterized in that they are Gram-positive bacteria are a preferred embodiment.

Gram-positive bacteria, such as, for example, bacilli or actinomycetes or other representatives of Actinomycetales, have no outer membrane so that secreted proteins are immediately released into the nutrient medium surrounding the cells, from which medium the expressed proteins of the invention can be directly purified according to another preferred embodiment.

Host cells which are characterized in that they belong to the genus Bacillus, preferably to the species Bacillus licheniformis, Bacillus amyloliquefaciens, Bacillus subtilis or Bacillus alcalophilus, are a preferred embodiment.

Said host cells are established Gram-positive expression systems.

Host cells which are characterized in that they belong to the order Actinomycetales, in particular to the genus Streptomyces, preferably to the species Streptomyces lividans, particularly preferably to S. lividans TK24, or to the species Streptomyces sp. 327* or Streptomyces sp. B400B, in particular to either of the two strains DSM 13990 or DSM 13991, are a preferred embodiment.

For particularly preferred embodiments of proteins of the invention were obtained from representatives of said genus and, respectively, said species, as described above and in the examples. Owing to the similar codon usage, said representatives are also particularly suitable for expressing the genes and gene fragments provided by the present invention.

They enable homologous protein expression in the case where they express their own genes. Said homologous protein expression may have the advantage that natural modification reactions in connection with translation are carried out on the protein being produced in the same way as they would also proceed naturally. This may be carried out, for example, via an introduced vector which introduces into said cells the already present endogenous gene or inventive modifications of the same, for example with multiple copies.

Host cells characterized in that they are Gram-negative bacteria are a preferred embodiment.

For most experience in biotechnological production has been obtained with Gram-negative bacteria such as, for example, E. coli or Klebsiella. These bacteria, moreover, secrete a multiplicity of proteins into the periplasmic space, i.e. the compartment between the two membranes enveloping the cells. This may be utilized for special applications. On the other hand, however, preference may also be given to the specific release of the enzymes produced by Gram-negative bacteria into the extracellular space. A method for this is described, for example, in the international patent application PCT/EP01/04227 with the title “Verfahren zur Herstellung rekombinanter Proteine durch gram-negative Bakterien” [Method for producing recombinant proteins by Gram-negative bacteria].

Preference is given to those host cells which are characterized in that they belong to the genus Escherichia, preferably to the species Escherichia coli, particularly preferably to any of the strains E. coli JM 109, E. coli DH100B or E. coli DH 12S.

Said strains are established, generally accessible laboratory and production strains. The strain E. coli DH 12S was used successfully for heterologous expression of the Actinomycetales genes obtained in example 6; this made it possible to obtain proteins with detectable α-amylase activity.

A further embodiment is host cells which are characterized in that they are eukaryotic cells, in particular those which posttranslationally modify the protein produced.

Examples of these are fungi or yeasts such as Saccharomyces or Kluyveromyces. This may be particularly advantageous, for example, if the proteins are intended to receive modifications specific in connection with their synthesis which are made possible by systems of this kind. They include, for example, binding of low-molecular weight compounds such as membrane anchors or oligosaccharides.

A further embodiment of this subject matter of the invention is methods for preparing a protein or derivative of the invention. For this purpose, nucleic acids of the invention are used, optionally using an appropriate vector and/or using an appropriate host cell or using a cell which produces said protein naturally. Among these, preference is given accordingly to the natural organisms discussed above.

Any of the elements already discussed above may be combined to methods in order to prepare proteins of the invention. In this connection, a multiplicity of possible combinations of method steps is conceivable for each protein of the invention. All of these implement the idea on which the present invention is based, namely to prepare quantitatively representatives of a type of protein defined via the amylolytic function and, at the same time, high homology to the sequences indicated in the sequence listings with the aid of the corresponding genetic information. In each actual individual case, the optimal method must be determined experimentally.

In principle, the procedure here is as follows: nucleic acids of the invention in DNA form are ligated into a suitable expression vector. The latter is transformed into the host cell, for example into cells of a readily culturable bacterial strain which exports the proteins whose genes are under the control of appropriate genetic elements into the surrounding nutrient medium or which accumulates said proteins inside the cell; elements regulating this may be provided, for example, by the expression vector. It is possible to purify the protein of the invention from the surrounding medium or, with disruption of the cells, from the host cells themselves via a plurality of purification steps such as, for example, precipitations or chromatographies. A skilled worker is capable of transferring a system which has been optimized experimentally on the laboratory scale to large-scale production.

The possible industrial uses for amylases of the invention are a separate subject matter of the invention, since the underlying object had been to provide suitable amylases for various industrial processes. The most important of these processes will be discussed below.

Numerous possible applications for amylolytic enzymes, which are established in industry, are discussed in manuals such as, for example, the book “Industrial enzymes and their applications” by H. Uhlig, Wiley-Verlag, New York, 1998. The following compilation is not to be understood as a final list but is a selection of the possible industrial uses. Should it turn out that individual proteins within the similarity range are, owing to their enzymic, i.e. amylolytic, properties, suitable for additional possible applications not explicitly claimed herein, said possible applications are hereby included in the scope of protection of the present invention.

In one embodiment, the present invention relates to detergents and cleaning agents which are characterized in that they comprise a protein or derivative of the invention.

An important field of use for amylolytic enzymes is that as active components in detergents or cleaning agents for cleaning textiles or solid surfaces such as, for example, dishes, floors or tools. In these applications, the amylolytic activity serves to hydrolytically dissolve or detach from the underlying material carbohydrate-containing soilings, in particular those based on starch. In this connection, said enzymes may be applied alone, in suitable media or else in detergents or cleaning agents. The conditions to be chosen for this, such as, for example, temperature, pH, ionic strength, redox ratios or mechanical influences, should be optimized for the particular cleaning problem, i.e. with respect to the soiling and the support material. Thus usual temperature ranges for detergents and cleaning agents are from 10° C. for manual agents via 40° C. and 60° C. up to 95° for machine agents or industrial applications. Preferably, the ingredients of the agents in question are also matched to one another. Since in modern washing machines and dishwashers the temperature is usually continuously adjustable, any intermediate temperatures are also included. The other conditions may likewise be designed in a very variable manner via the other components of said agents with respect to the particular cleaning purpose.

Preferred agents of the invention distinguish themselves by the washing or cleaning performance of said agent under any of the conditions definable in this way being improved by the addition of an amylolytic enzyme of the invention, in comparison with the formulation without said enzyme. In this respect, preference is given to incorporating those amylolytic proteins into agents of the invention which are capable of improving the washing and/or cleaning performance of a detergent or cleaning agent.

Further preferred agents distinguish themselves in that the amylolytic enzymes and the other components remove the soilings synergistically. This is carried out, for example, by other components of said agents, such as, for example, surfactants, solubilizing the hydrolysis products of the amylolytic proteins. A protein of the invention may be used both in agents for industrial consumers or industrial users and in products for the private consumer, with all presentations established in the prior art and/or convenient presentations also being embodiments of the present invention.

The amylases are combined in agents of the invention, for example, with individual or several of the following ingredients: anionic, cationic and/or nonionic surfactants, builders, bleaches, bleach activators, bleach catalysts, enzymes such as, for example, proteases, other amylases, lipases, cellulases, hemicellulases or oxidases, stabilizers, in particular enzyme stabilizers, solvents, thickeners, abrasive substances, dyes, fragrances, graying inhibitors, color transfer inhibitors, foam inhibitors, corrosion inhibitors, in particular silver protectants, optical brighteners, antimicrobial substances, UV protectants and other components known from the prior art.

Preferred agents are characterized in that they contain 0.000001 percent by weight to 5% by weight and, increasingly preferably, 0.00001 to 4% by weight, 0.0001 to 3% by weight, 0.001 to 2% by weight or 0.01 to 1% by weight of the amylolytic protein or derivative.

Preferred agents are characterized in that they comprise more than one phase. These may include solid agents, in particular those in which at least two different solid components, in particular powders, granules or extrudates, are present in an overall loose mixture or in which at least two solid phases are connected to one another, in particular after a joint compacting step. In addition, at least one of the phases may contain an amylase-sensitive material, in particular starch, or be, at least partially, surrounded or coated by said material.

Likewise preferred agents are characterized in that they are overall liquid, gel-like or paste-like. Preferably, the protein therein and/or at least one of the enzymes therein and/or at least one of the other components therein are present individually or encapsulated together with other components, preferably in microcapsules, particularly preferably in those made of an amylase-sensitive material.

In a preferred embodiment, the amylolytic activity is modified, in particular stabilized and/or increased in its contribution to the washing or cleaning performance of the agent, by any of the other components of the agent.

In accordance with these discussions, any washing or cleaning processes which are based in at least one part on an α-amylase of the invention or preferably an agent of the invention is used are also embodiments of the present invention.

Any use of an α-amylase of the invention or preferably of an agent of the invention for washing or cleaning textiles or hard surfaces is also an embodiment of the present invention.

Another embodiment is the use of a protein or derivative of the invention for the treatment of raw materials or intermediates in the manufacture of textiles, in particular for desizing cotton.

Raw materials and intermediates in the manufacture of textiles, for example of those based on cotton, are provided with starch during their production and further processing, in order to improve the finish. This method, which is applied to yarns, to intermediates and to textiles, is called sizing. Amylolytic proteins of the invention are suitable for removing the starch-containing protective layer (desizing).

A further embodiment is methods for starch liquefaction, in particular for ethanol production, which are characterized in that therein a protein or derivative of the invention is used.

For starch liquefaction, starch soaked in water or buffer is incubated with amylolytic enzymes, thereby cleaving the polysaccharide into smaller parts, in the end primarily into maltose. Preference is given to using for such a process or a part thereof enzymes of the invention, if they can be readily adapted to a corresponding production process, owing to their biochemical properties. This may be the case, for example, if they are to be introduced in one step in addition to other enzymes which require the same reaction conditions. Particular preference is given to amylolytic proteins of the invention if interest is focused especially on the products generated by said proteins themselves. Starch liquefaction may also be a step in a multistage process for producing ethanol or secondary products derived therefrom, for example acetic acid.

Another embodiment is the use of a protein or derivative of the invention for preparing linear and/or short-chain oligosaccharides.

Owing to their enzymic activity, amylolytic proteins of the invention form from starch-like polymers primarily higher molecular weight oligosaccharides such as, for example, maltohexaose, maltoheptaose or maltooctaose, after a relatively short incubation time. After a longer incubation time, the proportion of lower oligosaccharides such as, for example, maltose or maltotriose in the reaction products increases. If there is particular interest in certain reaction products, it is possible to use appropriate variants of proteins of the invention and/or to design the reaction conditions accordingly. This is particularly attractive if mixtures of similar compounds rather than pure compounds matter, as, for example, in the generation of solutions, suspensions or gels with only certain physical properties.

Another embodiment is the use of a protein or derivative of the invention for hydrolyzing cyclodextrins.

Cyclodextrins are α-1,4-glycosidically linked, cyclic oligosaccharides of which those consisting of 6, 7 and 8 glucose monomers, the α-, β- and γ-cyclodextrins, respectively (or cyclohexa-, -hepta- and -octaamyloses, respectively), are economically the most important. They may form inclusion compounds with hydrophobic guest molecules such as, for example, fragrances, flavorings or pharmaceutical active substances, from which inclusion compounds the guest molecules can be released again when required. Depending on the field of use of the ingredients, for example for food production, pharmacy or cosmetics, for example in appropriate products, such inclusion compounds are important to the final consumer. The release of ingredients from cyclodextrins is thus a possible use for proteins of the invention.

Another embodiment is the use of a protein or derivative of the invention for releasing low-molecular weight compounds from polysaccharide carriers or cyclodextrins.

Owing to their enzymic activity, amylolytic proteins of the invention may liberate low-molecular weight compounds also from other α-1,4-glycosidically linked polysaccharides. This may take place, as for the cyclodextrins, at the molecular level as well as in larger systems such as, for example, ingredients encapsulated in the form of microcapsules. Starch, for example, is a material established in the prior art in order to encapsulate compounds such as, for example, enzymes, which are intended to be introduced in defined amounts into reaction mixtures, during storage. The controlled process of release from such capsules may be assisted by amylolytic enzymes of the invention.

Another embodiment is the use of a protein or derivative of the invention for producing food and/or food ingredients.

Likewise, the use of a protein or derivative of the invention for producing animal feed and/or animal feed ingredients is an embodiment of the present invention.

Wherever starch or starch-derived carbohydrates play a part as food or animal feed ingredients, an amylolytic activity may be employed in producing these items. Said activity increases the proportion of monomers or oligomers compared to the polymeric sugar, possibly benefiting, for example, the taste, digestability or consistency of the food product. This may be required for producing particular animal feed but also, for example, in the production of fruit juices, wine or other food products, if the proportion of polymeric sugars is to be reduced and that of sweet and/or readily soluble sugars is to be increased. The possible use discussed further above for starch liquefaction and/or ethanol production may be regarded as the industrial variant of this principle.

Amylases in addition also counteract the loss of taste, known as staling, of bakery products (antistaling effect). For this purpose, they are conveniently added to the dough before baking. Thus, preferred embodiments of the present invention are those in which proteins of the invention are used for making bakery products.

Another embodiment is the use of a protein or derivative of the invention for dissolving starch-containing adhesive bonds.

Temporary bonding processes which are characterized in that a protein or derivative of the invention is used therein are also an embodiment of the present invention.

In addition to other natural substances, starch has also been used as binder in paper production and bonding of different papers and cardboards already for centuries. This relates, for example, to drawings and books. Over the course of long periods of time, unfavorable influences such as, for example, moisture can cause such papers to become wavy or to break, leading possibly to complete destruction thereof. Restoration of such papers and cardboards may require dissolution of the adhesive layers, which is facilitated considerably by using an amylolytic protein of the invention.

Plant polymers such as starch or cellulose and their water-soluble derivatives are used, inter alia, as adhesives or pastes. For this purpose, said polymers must first swell in water and then, after application to the material to be glued, dry, thus attaching said material to the base. The enzyme of the invention may be added to such an aqueous suspension in order to influence the adhesive properties of the resulting paste. However, it may also be added to the paste instead of or in addition to said function in order to stay, after drying, on the material to be glued in an inactive manner for a long time, for example several years. Changing the environmental conditions specifically, for example by wetting, may then be used in order to activate the enzyme at a later time and thus cause the paste to dissolve. In this way it is possible to detach again the glued material more readily from the base. In this method, the enzyme of the invention acts, owing to its amylolytic activity, as separating agent in a temporary bonding process or as “switch” for detaching the glued material.

### EXAMPLES

### Example 1

### Construction of Primer Oligonucleotides for Amplifying Variable Sequence Regions of α-Amylases

Based on comparisons of known sequences of α-amylases (E.C. 3.2.1.1) of Gram-positive eubacteria of various genera of the order Actinomycetales (Streptomyces, Thermomonospora, etc.) from GenBank (National Center for Biotechnology Information NCBI, National Institutes of Health, Bethesda, Md., USA; updated November 1998), potentially conserved sequence regions were identified which flank sequence regions as variable as possible. The conserved sequence blocks identified were the blocks A to E highlighted in FIG. 1. They extend over the following amino acid positions: A: 58-91, B: 94-141, C: 155-207, D: 295-345, E: 392-427. On the basis of the sequences of these regions, the oligonucleotides compiled in table 4 were designed as primers of the invention; their sequences are indicated in the sequence listing under numbers 9 to 33. Their position relative to the conserved sequence blocks A to E within an α-amylase gene is illustrated in FIG. 1 by way of the example of Streptomyces griseus α-amylase (GenBank Accession number X57568). They can be distinguished by their orientation to said gene into forward primer and reverse primer.

TABLE 4
PCR primers for amplifying partial α-amylase sequences
of representatives of the order Actinomycetales

| Name | SEQ ID NO. | Orientation |
|---|---|---|
| GEX015 | 13 | forward |
| GEX016 | 14 | forward |
| GEX017 | 15 | forward |
| GEX018 | 16 | forward |
| GEX019 | 17 | reverse |
| GEX020 | 18 | reverse |
| GEX021 | 19 | reverse |
| GEX022 | 20 | reverse |
| GEX023 | 21 | forward |
| GEX024 | 9 | forward |
| GEX025 | 22 | reverse |
| GEX026 | 10 | reverse |
| GEX027 | 23 | forward |
| GEX028 | 24 | forward |
| GEX029 | 11 | forward |
| GEX030 | 25 | reverse |
| GEX031 | 12 | reverse |
| GEX036 | 26 | forward |
| GEX037 | 27 | forward |
| GEX038 | 28 | reverse |
| GEX039 | 29 | reverse |
| GEX040 | 30 | forward |
| GEX041 | 31 | forward |
| GEX042 | 32 | reverse |
| GEX043 | 33 | reverse |

Indicated are in each case the names used in FIG. 1, the corresponding numbers according to the sequence listing of the present patent application and the orientation of the primers relative to the amylase genes in question.

The templates used were genomic DNA preparations, obtained according to standard methods, of known and unknown bacterial isolates from various soil samples, which were present in each case as pure culture. The primers were used in combinations of in each case a forward primer with a reverse primer in each PCR. The PCR products were fractionated via agarose gel electrophoresis, as described below, and used for screening for complete genes.

The PCR reaction mixtures included those containing the primer combinations GEX024 (forward)/GEX026 (reverse) and GEX029 (forward)/GEX031 (reverse). These were derived from the sequence regions C (GEX024 and GEX029) and D (GEX026 and GEX031) (FIG. 1), which correspond to the amylase domains β4 and β7 of the (αβ)₈ barrel structure, as they are defined in the article “Alpha-Amylase family: molecular biology and evolution” (Janecek, S. (1997), Prog. Biophys. Mol. Biol. 67 (1), pp. 67-97). The nucleotide sequences of these four primers are indicated in the sequence listing under SEQ ID NO. 9 to SEQ ID NO. 12.

These four primers produced in the PCR mixtures with the isolated nucleic acids of bacterial isolates reproducibly sized fragments. FIG. 2 depicts an exemplary size analysis of the amplification products obtained of 20 randomly selected preparations of isolates of the order Actinomycetales, using the primer pair GEX024/GEX026 in FIG. 2A and the primer pair GEX029/GEX031 in FIG. 2B. The PCR conditions are indicated in example 2; after the reaction, in each case 1/10 of the reaction volume was fractionated in a 2.5% strength agarose gel. The reaction mixtures are based on the Streptomyces sp. strains compiled in the following table.

TABLE 5
Streptomyces sp. strains whose PCR products with the primer
pairs GEX024/GEX026 and GEX029/GEX031 are depicted in FIG. 2.

| Lanes | Streptomyces sp. | SEQ ID NO. |
|---|---|---|
| 1 | B101A | 45 |
| 2 | B114C | 83 |
| 3 | B134 | 97 |
| 4 | B135A | 98 |
| 5 | B138A | 101 |
| 6 | B152A | 108 |
| 7 | B153(B) | 109 |
| 8 | B161A | 116 |
| 9 | B156B | 111 |
| 10 | B157C | 112 |
| 11 | B158A | 113 |
| 12 | B160B | 115 |
| 13 | B161A | 116 |
| 14 | B373 | 232 |
| 15 | B375 | 234 |
| 16 | B380 | 236 |
| 17 | B390 | 238 |
| 18 | B392A | 239 |
| 19 | B392A | 239 |
| 20 | B394 | 241 |

Both primer pairs were used to amplify DNA fragments corresponding to a 300 bp band. A fragment of approx. 500 bp additionally appeared in some reaction mixtures. Both products were identified as α-amylase sequences by sequencing (see example 2). The other primers, listed above and in the sequence listing, showed lower selectivity.

All strains listed in the sequence listing produced the same results; in particular, the 300 bp band was reproducible for all strains. All fragments, in particular the 300 bp fragments, that we were able to obtain in this way from the samples assayed can be homologized. They correspond, in particular in the primer regions, to a large extent, albeit not necessarily completely, and this was sufficient in order to identify them by the method of the invention. They define, via their sequence variations to one another, as indicated also in the sequence listing under SEQ ID NO. 263 and in FIG. 3, a sequence variance space within the group of amylolytic proteins.

### Example 2

### Obtainment of Genomic DNA from Actinomycetes and PCR-Based Amplification and Cloning of α-Amylase Gene Sequences

The applicability of the method of the invention was tested on a collection of several hundred individual isolates of the order Actinomycetales, which had been obtained from soil samples according to common micro-biological methods. They were in each case cultured individual strains.

In this example, the isolation was carried out by standard methods, starting from soil samples, using GYM medium (4.0 g/l glucose, 4.0 g/l yeast extract; 10 g/l malt extract, 2.0g/l CaCO₃, 10 g/l agar, pH 7.2) with addition of 50 μg/ml nystatin to suppress the accompanying flora.

Strain Cultivation

The actinomycetes strains were grown by inoculating 5 ml of GHPF medium (10 g/l glucose; 5 g/l peptone from casein; 5 g/l meat extract; 5 g/l yeast extract; 0.74 g/l CaCl₂×2H₂O; pH 7.2) containing 50 μl of spore suspension at the in each case strain-specific temperature (28° C. to 50° C.) and a shaker rate of 200 rpm.

After sufficient amounts of mycelium had formed, 200 ml of YEME medium (3 g/l yeast extract; 5 g/l bacto tryptone; 3 g/l malt extract; 10 g/l D(+)glucose; 340 g/l sucrose; pH 7.2; after autoclaving, addition of 5 mM MgCl₂ and glycine, strain-specific 0.5-20 g/l) were inoculated with the entire preculture and incubated with shaking at 170 rpm in a 1-liter flask with baffles at the same temperature and for 3 days.

DNA Isolation

To prepare DNA, in each case 1 g of mycelium washed in 20% (v/v) glycerol was resuspended in 3 ml of TE25S buffer (25 mM Tris, pH 8.0; 25 mM EDTA; 0.3 M sucrose) with addition of 2 mg/ml lysozyme, inverted at 37° C. for 30 min and, after addition of 4 ml of 2× Kirby mix (2% SDS; 120 g/l sodium 4-aminosalicylate; 100 mM Tris, pH 8.0; 6% phenol, saturated with 50 mM Tris/pH 8.0 and supplemented with 0.1% hydroxyquinoline), inverted for a further 10 min. This was followed by extracting the aqueous phase twice with, successively, 8 and 3 ml of phenol/chloroform/isoamyl alcohol (25:24:1) by inverting in each case for 10 minutes and subsequently centrifuging at 2 500×g. The DNA was precipitated by adding 1/10 volume of 3M sodium acetate (unbuffered) and 0.6 volume of isopropanol with gentle inverting, spooled and purified five times with 70% ethanol. The pellet was taken up in 3 ml of HE buffer (10 mM HEPES, pH 8.0 with NaOH; 1 mM EDTA), incubated at 50-55° C. for 1 h and dissolved at 4° C. overnight.

Where appropriate, this may be followed by another purification step in order to remove components which may accompany the DNA and inhibit an amplification reaction. This was necessary for some DNA preparations in which initially no PCR product had formed (see below). In this case, the influence of inhibitory components in the DNA preparation had to be stopped by diluting (up to 1:100) or by purification via a QIAquick PCR purification column (Qiagen, Hilden, Germany) according to the manufacturer's instructions.

On the following day, the DNA was worked up by incubating at 37° C. successively with 40 μg/ml RNaseA (heat-inactivated) and 0.5 mg/ml proteinase K, in each case for one hour. Subsequently, the aqueous phase was extracted in the same volume as phenol/chloroform/iso-amyl alcohol (25:24:1), inverted for 10 min, centrifuged at 2 500×g and precipitated as on the previous day, spooled, then washed and dissolved in 1 ml of HE buffer.

Amplification of the Amylase Sequences by the PCR of the Invention

Prior to characterizing the amylase sequences, the latter were first amplified by the polymerase chain reaction (PCR) which was carried out using HotStar Taq polymerase (Qiagen, Hilden, Germany) and the amylase-specific primers (example 1, table 3). For this purpose, in each case 4 μl of the genomic DNA isolated by the above method were used in the presence of 200 μM dNTPs (each of DATP, dTTP, dCTP, dGTP), 20 pmol of in each case a forward primer and a reverse primer, 2.5 U of HotStar Taq polymerase (Qiagen, Hilden, Germany) and 1 x PCR buffer were used in a total volume of 50 μl. Fragments of from 280 to 500 bp in size were amplified in the following cycles: 1 cycle at 95° C. for 15 min; 40 cycles at, in each case, 95° C. for 30 s, 60° C. for 30 s and 72° C. for 30 s; 1 cycle at 72° C. for 7 min.

The primer pair GEX024/GEX026, in particular, produced constant results for all genomic DNAs with respect to a 300 bp product which exhibited homologies to amylases in the sequencing (see example 1). In some DNA preparations, initially no product was formed. In this case, the influence of inhibitory components in the DNA preparation had to be stopped by diluting (up to 1:100) or by purification via a QIAquick PCR purification column (Qiagen, Hilden, Germany) according to the manufacturer's information.

The bands (approx. 300 bp) obtained with the primer combination GEX024/GEX026 were sequenced by eluting them with the aid of the QIAEX II® kit from Qiagen (Hilden, Germany) and cloning them by means of the TOPO TA® kit (Invitrogen, Groningen, The Netherlands) into the pCR2.1-TOPO® vector, according to the manufacturer's instructions. The ligation products were used to transform E. coli-TOP® 10F (Invitrogen, Groningen, The Netherlands). After blue/white selection of recombinant clones on LB medium containing ampicillin (100 μg/ml), IPTG (100 mM), X-Gal (40 mg/ml), the plasmid DNA of white clones was checked, after mini preparation, for DNA insert in a restriction analysis with EcoRI.

The sequences of plasmids with DNA insert were analyzed in a routine manner in an ABI PRISM® 310 (Perkin Elmer, Weiterstadt, Germany) according to the manufacturer's instructions by means of AmpliTaq-FS-Big-Dye® terminator kit (Perkin Elmer, Weiterstadt, Germany) using 200-500 ng of plasmid DNA, 10 pmol of primer (TopoF: 5′-GCTCGGATCCACTAGTAACG-3′ and TopoR: 5′-CTCTAGATGCATGCTCGAG-3′), 4 μl of pre-mix in a total volume of 20 μl. The DNA sequences obtained were routinely and conceptionally transcribed into an amino acid sequence, compared via the BlastX, BlastN and BlastP algorithms with the GenBank entries available and identified in this way as partial sequences of α-amylase genes. Table 1 indicates for each partial sequence obtained the amylolytic proteins entered in publicly accessible databases, which have in each case the highest homology to the sequences in question. The partial sequences found are listed in the sequence listing under numbers SEQ ID NO. 2, 4 and 34 to 262 and combined in the consensus sequence of FIG. 3 and SEQ ID NO. 263, respectively.

Special mention should be given to the partial sequences which were likewise amplified using the primer pair GEX024/GEX026 and which are depicted in the sequence listing under numbers SEQ ID NO. 1 to SEQ ID NO. 4. The corresponding strains were denoted Streptomyces sp. B327* (SEQ ID NO. 1 and 2) and Streptomyces sp. B400B (SEQ ID NO. 3 and 4). The partial sequence obtained from the Actinomycetales isolate Streptomyces sp. B327* comprises, including the primer regions, 302 base pairs and codes for 100 amino acids. The partial sequence obtained from the Actinomycetales isolate Streptomyces sp. B400B comprises, including the primer regions, 293 base pairs and codes for 97 amino acids.

### Example 3

### Typing of Microbial Pure Culture Isolates and Cloning of α-Amylase Gene Sequences

The DNA sequences of α-amylases have been amplified, as described, from all of the streptomycetes strains referred to in example 2 by a PCR of the invention using the primers GEX024/GEX026 and then cloned and sequenced. Thus, in total, 231 partial sequences of α-amylases were found which are listed in the sequence listing under numbers SEQ ID NO. 2, 4 and 34 to 262. The individual Streptomyces sp. strains referred to therein, for example Streptomyces sp. B327* and Streptomyces sp. B400B, can be characterized due to the fact that it is possible to amplify therefrom fragments having an appropriate size and the partial amylase sequences indicated in the sequence listing via a PCR using the primer pair GEX024/GEX026.

A consensus sequence was derived from said sequences with the aid of the alignment program Clustal X®, Version 1.64 b (standard settings; described in: Thompson, J. D., Higgins, D. G. and Gibson, T. J. (1994), “CLUSTAL W: improving the sensitivity of progressive multiple sequence alignment through sequence weighting, position specific gap penalties and weight matrix choice”, Nucleic Acids Res., volume 22, pp. 4673-4680). They may be regarded, via this common sequence, as representatives of a particular type of amylase. Said consensus sequence is displayed in two alternative representations in FIG. 3 and in the sequence listing under SEQ ID NO. 263. The position of said consensus sequence was defined on the basis of the sequence of the PCR product of strain B327* with the primer pair GEX024/GEX026 (100 amino acids; SEQ ID NO. 2).

The amino acid positions 1 to 8 and 93 to 100 which appear to be practically invariable in the consensus sequence are prescribed by the PCR primer sequences (GEX024/GEX026). Owing to the selectivity of the PCR performed, only slight deviations were found within these regions. More stringent conditions (in particular higher annealing temperature) result in a lower number of positive results, namely those sequences which are in complete agreement with the primers in the primer regions. In contrast, owing to the complexity of the starting DNA, products which are more difficult to separate in agarose gels are increasingly produced under less stringent conditions. Among said products, the proportion of sequences no longer coding for amylases increases.

Otherwise, only the amino acid residues histidine in position 11 and leucine in position 75 are conserved in the sequences listed across all amylase fragments assayed. Some fragments contain in position 28 sequences of a plurality of amino acids rather than a single amino acid; these are also indicated in FIG. 3 and SEQ ID NO. 263 and, respectively, the individual sequences in question. Said individual sequences are the partial sequence TAGGE of the strain Streptomyces sp. B347 (SEQ ID NO. 212), the partial sequence TSGAE of the strain Streptomyces sp. B361 (SEQ ID NO. 204) and the partial sequence YPAWGGGK of the strain Streptomyces sp. B4006B (SEQ ID NO. 246). In addition, some fragments do not contain any amino acid in positions 29, 45, 46, 66, 70, 81, 82, 83 or 91 so that the individual numbering thereof deviates slightly from that of the consensus sequence; these fragments include, for example, those of the strains Streptomyces sp. B347, B361 and B1059.

The sequences shown have, as can be found in table 1 already shown hereinbefore, between about 45% and 97% sequence identity to known α-amylases. These sequences, too, partly exhibit a considerable variance. Thus, the amino acid sequences found of the α-amylases from Streptomyces sp. B327* and B400B are merely 57% identical to one another.

### Example 4

### Detection of Full-Length Gene Sequences Coding for α-Amylase by Means of Activity Screening of Expression Gene Banks

Complete α-amylase genes were cloned and isolated from Actinomycetales by choosing expression cloning in the heterologous host organism Escherichia coli, namely the strain DH 12S. The promoter used was the IPTG (isopropylthiogalactoside)-inducible β-galactosidase promoter of the lac operons (lac promoter). Said promoter was provided on the pUC18 plasmid vector (GenBank (National Center for Biotechnology Information NCBI, National Institutes of Health, Bethesda, Md., USA); accession number L08752; FIG. 4).

Preparation of Expression Gene Banks for the Particular Strains

Purified genomic DNA from the particular Actinomycetales strains, which had been obtained according to example 2, was partially cleaved with the restriction enzyme Aci I, and the size range from 3 to 5 kb was ligated into pUC18 plasmid vectors linearized with the restriction enzyme Acc I (FIG. 4). A desired 5-fold covering of the genome in a gene bank, with an average insert size of 4 kb and an estimated actinomycete genome size of 8 Mb, results in a figure of 20 000 primary clones. Since expression starts from the lac promoter located on the plasmid but the inserts can integrate in each case in two orientations, this figure is increased to at least 40 000 clones, which was the minimum number of clones obtained for each gene bank.

The optimal restriction parameters for the preparative partial digestion of genomic DNA from actinomycetes were determined by first carrying out restriction kinetics using 6 μg of DNA and restriction enzyme (AciI) at concentrations of from 0.1 to 0.4 U per μg of DNA in 1 × buffer, where appropriate according to the manufacturer's instructions with bovine serum albumin (BSA), in a total volume of 15 μl. For this purpose, the reaction mixtures were, initially without enzyme, heated to 37° C. Thereafter, the particular reaction was started by adding restriction endonuclease. At fixed intervals of between 0 and 10 min, in each case 1.5 μl of reaction mixture was added to 1× stop buffer (6×: 10 mM Tris, pH 7.0; 20% glycerol; 0.1% SDS) on ice and analyzed on a 0.7% strength agarose gel, thereby determining individually for each DNA preparation the optimal restriction time for a partial digest, which was on average 4 to 5 min.

The preparative partial digestion was carried out as described above, but with ten times the amount of reaction mixture using 60 μg of chromosomal actinomycete DNA and the previously optimized amount of enzyme for the period which was in each case determined individually. After stopping the reaction by adding 1× stop buffer, the mixture was electrophoretically fractionated on a 0.7% strength agarose gel, the gel region containing DNA of 3-5 kb in size was excised and isolated in dialysis tubes at 4° C. by electroeluting for two hours. The DNA was precipitated with 1/10 volume of 3 M sodium acetate and 2.5 volumes of ethanol, taken up in a smaller volume and then, for further removal of smaller fragments, subjected to a second gel electrophoresis with subsequent electroelution and another concentration.

Ligation with the plasmid vector pUC18 was carried out at 16° C. overnight in a total volume of 20 μl by using 150 ng of Acc I-linearized pUC18 vector which had been dephosphorylated with CIAP (alkaline phosphatase from calf thymus) according to the manufacturer's instructions and 450 ng of partially cleaved genomic DNA, and also an appropriate amount of ligase (here 400 NEB units) in 1× ligase buffer.

Competent E. coli DH 12S cells (Gibco Life Technologies, Karlsruhe, Germany) were transformed via electrotransformation. To this end, 1 μl of ligation mixture and 30 μl of cells were mixed, incubated on ice in an electroporation cell for 1 min and treated in an electroporator (BTX® ECM399, Genetronics Inc., San Diego, Calif., USA) according to the manufacturer's instructions. After immediate transfer to 1 ml of SOC medium (2% bacto tryptone; 0.5% yeast extract; 10 mM NaCl; 2.5 mM KCl; pH 7.0, adjusted with NaOH; autoclaved; supplemented with 10 mM MgSO₄ and MgCl₂ and 20 mM D(+)glucose), there was a resting phase of 1 h at 37° C. prior to plating.

The quality of the gene bank was tested by determining the total number of primary transformants generated and the number of insert-carrying clones via blue/white selection in a test plating. To this end, 1 and 10 μl of the ligation mixture were in each case plated out on LB medium containing ampicillin, IPTG, X-Gal (as described above) and incubated at 37° C. overnight. The actually cloned insert sizes were confirmed by isolating the plasmids of at least 10 white colonies of the test plating and carrying out a suitable restriction digest with subsequent gel-electrophoretic size analysis.

Activity Assay of the Gene Banks

First, the ability of the gene banks to break down starch was determined. To this end, LB agar containing 1% soluble starch (Merck, Darmstadt, Germany, Cat. No. #1252) was used on which clones capable of breaking down starch can be identified by lysis halos. This required the plates to be stored at 4° C. for from two to four weeks, until said plates became cloudy, after which breakdown of starch was visible as a clearing up of said plates around the colonies. Additionally, soluble starch (approx. 0.6% w/v) labeled with Cibacron Brilliant Red® 3B-A (Aldrich, Taufkirchen, Germany) was used (Biely, P., Mislovicova, D., Markovic, O., Kalac, V. (1988) : “A new chromogenic substrate for assay and detection of alpha-amylase”; Anal. Biochem., volume 172 (1), pages 176-179), breakdown of starch being detectable by a fading of the reddish agar in the area of the positive clone. Prior to use, the plates were admixed in both cases with IPTG (100 mM) for inducing the promoter and with ampicillin (100 μg/ml) for exerting selection pressure on the transformants.

According to the titer of the bank generated in each case, a defined volume of the transformation mixture containing approx. 10 000 colonies per plate (14 cm in diameter) was plated out evenly by means of glass beads (primary plating). After incubating at 37° C. for 16 hours, the plates were incubated at 28° C. for amylase expression and release for another 24-48 h. The incubation at 28° C. was usually indispensable for visualizing a breakdown of starch. Improved protein folding and permeabilization of the outer cell membrane may play a part here (Stathopoulos, C., Georgiou, G., Earhart, C. F. (1996): “Characterization of Escherichia coli expressing an Lpp′ OmpA(46-159)-PhoA fusion protein localized in the outer membrane”; Appl. Microbiol. Biotechnol., volume 45 (1-2), pages 112-119). This was thus accompanied by a release of the α-amylase produced in each case, and a cell lysis, to be carried out additionally, for detecting cytoplasmic amylases which had not been exported was not required.

After isolating the colonies from the halo regions of the primary plating by renewed (secondary) plating on the same starch plates, amylase-forming single colonies were selected on the basis of renewed halo formation. For photo-archiving purposes, staining of the starch plates, after securing the clones, with 50% lugol solution (Merck, Germany) for unambiguous visualization of starch-free regions (breakdown halos) has proved useful.

FIG. 7 depicts an appropriately stained, starch-containing LB_{Amp}-agar plate to which in each case suspensions of positive clones of the secondary plating have been applied in drops.

Isolation of Individual Clones

The plasmid DNA of positive clones, obtained after minipreparation (kit from Qiagen, Hilden, Germany), used according to manufacturer's instructions) was studied for insert size in a restriction analysis with Eco R I or Sac I/Hind III and then sequenced. Here, the insert-flanking M13 primers (M13 forward: 5′-GTAAAACGACGGCCAG-3′; M13 reverse: 5′-CAGGAAACAGCTATGAC-3′) were used first in order to obtain finally the entire sequence via “primer walking” as known from the prior art. The principle of this procedure is to derive new sequencing primers in a repeating sequence, starting from determined sequences and to use said primers for sequencing the adjoining, still unknown regions from which in turn the next sequencing primers can be derived.

The complete DNA sequences and amino acid sequences derived therefrom, obtained in this way for the two Actinomycetales isolates Streptomyces sp. B327* and Streptomyces sp. B400B isolates (see example 2) are depicted in the sequence listing under SEQ ID NO. 5 and 6 and, respectively, SEQ ID NO. 7 and 8.

FIG. 5 depicts an alignment of the two complete α-amylases of Streptomyces sp. B 327 and Streptomyces sp. B400B with the amylase sequences most similar thereto, which were found in GenBank (National Center for Biotechnology Information NCBI, National Institutes of Health, Bethesda, USA) on 1.24.2001. The latter sequences are on the one hand Streptomyces albus α-amylase, accession number U51129; this is 80% identical across the entire protein to Streptomyces sp. B327*. On the other hand, Streptomyces sp. α-amylase (GenBank Acc. No. U8602) is 76.8% identical across the entire protein to Streptomyces sp. B400B.

### Example 5

Heterologous Expression of α-Amylases in Prokaryotic Expression Systems

In order to characterize the biochemical properties of the cloned α-amylases, the latter should be produced in larger quantities and in soluble form. Said characterization was carried out for the complete amylase genes of Streptomyces sp. 327*, Streptomyces sp. B400B and the Actinomycetales isolate Streptomyces sp. B327B. The expression host used was Streptomyces lividans TK24; the expression vector used was pAX5a (FIG. 6) in which expression is under the control of the constitutive ermE promoter.

To introduce the Spe I and Eco RI cleavage sites required for cloning and to replace the relatively rare and therefore possibly relatively poorly translated start codons of the amylase genes of B327* (GTG) and B400B (TTG) with ATG, a PCR with appropriately modified primers was carried out. Thus, for example, the forward primer: 5′ aaaactagtAAGGAGAACCCCCACaTGATATCGAG 3′ and the reverse primer: 5′ gaattcgcccttTCAGCAGTTGGCCTTGCCCG 3′ were used for cloning the complete α-amylase of Streptomyces sp. 327*. New, noncomplementary nucleotides are represented in lower case here. In the forward primer, the recognition site for the restriction enzyme Spe I and the start codon are underlined, with the Shine-Dalgarno sequence being highlighted in bold type. In the reverse primer, the recognition site for the restriction enzyme Eco RI is underlined and the stop codon (in reverse orientation) is highlighted in bold type. The genomic clones isolated from the gene banks served as templates.

After subcloning the PCR products generated with Pfx Platinum polymerase in the vector pCR Blunt II TOPO (Invitrogen, Groningen, the Netherlands), directed insertion into the Streptomycete expression vector pAX5a was carried out, using the Spe I and Eco RI restriction cleavage sites. This was followed by protoplast transformation of S. lividans TK 24 according to Kieser et al. (Kieser, T., Bibb, M. J., Buttner, M. J., Chater, K. F., Hopwood, D. A. (2000): “PEG-assisted transformation of Streptomyces protoplasts with plasmid DNA. Rapid small-scale procedure.” in: “Practical Streptomyces Genetics”, The John Innes Foundation (publisher), Crows, Norwich, England) and plating out the transformants on the selective regeneration medium R2YE. Said medium consists of: 103 g of sucrose, 0.25 g of K₂SO₄, 0.12 g of MgCl_{2×6}H₂O, 10 g of glucose, 100 mg of Difco casaminoacids (Difco, Heidelberg, Germany), 10 ml of 0.5% strength (w/v) KH₂PO₄ solution, 80 ml of 3.68% strength (w/v) CaCl₂ solution, 1.5 ml of L-proline, 100 ml of TES buffer (pH 7.2; 2-{[Tris(hydroxymethyl)-methyl]amino}ethanesulfonic acid), 0.2 ml of trace element solution (40 mg/l of ZnCl₂, 200 mg/l of FeCl₃×6 H₂O, 10 mg/l of CuCl₂×2 H₂O, 10 mg/l of MnCl₂×2 H₂O, 10 mg/l of Na₂B₄O₇10 H₂O, 10 mg/l of (NH₄)6 MO₇O₂₄×4 H₂O) and 5 ml of 10% strength (w/v) Difco yeast extract (Difco, Heidelberg, Germany) to 1 l [lacuna] H₂O) containing 75 μg/ml of thiostreptone as selective antibiotic.

After replating to NBSA indicator plates (8 g/l of Nutrient Broth Difco, 15 g/l of Agar Serva, 1.5% (w/v) of soluble starch Merck #1252, pH 8.0; manufacturer: Difco, Heidelberg, Germany; Serva, Heidelberg, Germany; Merck, Darmstadt, Germany) and identification of amylase-positive clones which were recognized on the basis of the developing starch breakdown halos, cultivation in liquid culture to obtain enzymically active supernatants was carried out. For this purpose, a 5-ml preculture and, subsequently, a 50-ml main culture were cultured in 300-ml Erlenmeyer flasks with baffles by using GYM medium (4 g/l glucose, 4 g/l yeast extract, 10 g/l malt extract, 2 g/l CaCO₃) containing 75 μg/ml thiostreptone for 16 h and 4 days, respectively. The amylase-containing culture supernatant was obtained by removing the cell mass by centrifugation at 2 500 g for 15 min and subsequent filtration (0.45 μμm) and immediately subjected to measurement or stored, after shock-freezing in liquid nitrogen, in a freezer at −20° C.

### Example 6

### Characterization of the Amylolytic Activity of α-Amylase Representatives

The detection in principle of the amylolytic activity of the two α-amylases of the invention from Streptomyces sp. B327* and B400B has already been described in example 4. Here, the enzymic properties of enzymes prepared heterologously according to example 5 will be studied in more detail.

Characterization of the Enzymic Properties

The characterization of the enzymic properties, which may be the basis for application-related selection of enzymes, is carried out with regard to the large variety of fields of application of α-amylases. The enzymes may be prepared here by cloning and heterologous expression of the gene sequences according to examples 4 and 5 and the procedure illustrated above.

The amylolytic activity was determined by means of the dinitrosalicylic acid assay (DNSA assay). Here, hydrolysis of a complex starch substrate is determined on the basis of the increase in reducing ends of the polysaccharide, the absorption at 540 nm of unreacted DNSA reagent serving as measure for the hydrolytic activity.

The assay was carried out by introducing into the sample and blank wells in a 96-well plate for PCR thermocyclers (0.2 ml “thin-wall plate” #3416, MPB (Molecular BioProducts, San Diego, USA) in each case 25 μl of substrate solution (1% soluble starch Merck p.a. in 180 mM Tris-maleate buffer, pH 8.6 or in 180 mM corresponding assay buffer) and preincubating at 50° C., or at the particular assay temperature, in a thermocycler block for 5 min. After adding 20 μl of enzyme solution per well, the substrate was converted at 50° C., or at the particular assay temperature, for 15 min. After adding 65 μl of DNSA reagent (8.8 g of dinitrosalicylic acid, 250 g of potassium sodium tartrate, 6.3 g of sodium disulfite, 334 ml of 4.5% (w/v) NaOH, 915 ml of H₂O) to the sample wells and blank wells and adding 20 μl of enzyme solution to the blank wells, the assay plate was immediately transferred to a thermoblock preheated to 100° C. and incubated at 100° C. for 20 min. The samples were measured by transferring in each case 60 μl of the assay mixture to 200 μl of H₂O which had previously been introduced to a 96-well measuring plate (PS microplate, 96 well #655101, Greiner) and subsequently determining absorption at 540 nm by means of a microtiter-plate spectrophotometer (Spectramax 190, Molecular Devices, Sunnivale, USA), using H₂O as reference. In each case three measurements were carried out; the evaluation was carried out by subtracting the average of the 3 blank wells from the average of the 3 sample wells.

Temperature Stability and Temperature Profile

The temperature stability was determined by preincubating the solutions of recombinantly obtained α-amylases at various temperatures for 15 min and subsequently determining the remaining activity according to the above-described procedure at 50° C. and 100 mM Tris-maleate, pH 8.6. The maximum stability of α-amylase from Streptomyces sp. B327B was found to be at 45° C., but the effect of elevated temperatures up to 61° C. was relatively moderate, with remaining activities of more than 80%. The other results are compiled in table 6, with the optimum of 45° C. set to 100%.

TABLE 6
Temperature stability of α-amylase from Streptomyces sp. B327*

| | Remaining activity, |
|---|---|
| Preincubation temperature | relative to that of 45° C. |
| [° C.] | [%] |
| 45 | 100 |
| 50.4 | 94 |
| 55.8 | 86 |
| 61 | 84 |
| 64.7 | 46 |

The temperature profile was determined by carrying out the reaction according to the above-described procedure at various temperatures and in 100 mM Tris-maleate buffer, pH 8.6. The maximum activity of α-amylase from Streptomyces sp. B327* was at 41.3° C. Higher temperatures resulted in distinct losses of activity in this enzyme (see table 7). In contrast, the activity of Streptomyces sp. B327B amylase was relatively constant over the temperature range assayed (see table 8).

TABLE 7
Temperature profile of α-amylase from Streptomyces sp. B327*

| | Activity, relative to that |
|---|---|
| Temperature [° C.] | of 41.3° C. [%] |
| 40 | 87 |
| 41.3 | 100 |
| 50.7 | 38 |
| 56 | 38 |
| 59.8 | 35 |

TABLE 8
Temperature profile of α-amylase from Streptomyces sp. B327B

| | Activity, relative to that |
|---|---|
| Temperature [° C.] | of 41.3° C. [%] |
| 40 | 103 |
| 41.3 | 100 |
| 50.7 | 117 |
| 56 | 92 |
| 59.8 | 83 |

It is evident that the α-amylase of Streptomyces sp. B327B is more stable than that of Streptomyces sp. B327* over a broader temperature range and up to higher temperatures. The amylolytic enzymes provided by the present invention thus distinguish themselves by a considerable viability with respect to their temperature sensitivity.

Stability to pH Fluctuations

The activity of the α-amylase amylases was determined under various pH conditions by mixing the starch substrate solution in a pH range from 6.0 to 8.6 with the correspondingly adjusted 180 mM Tris-maleate buffer and, for pH 8.6 and higher, with 180 mM glycine-NaOH buffer. After diluting the substrate solution, the desired pH values and buffer concentrations of 100 mM Tris-maleate buffer and 100 mM glycine-NaOH buffer, respectively were obtained under assay conditions. Influences of the buffer system were canceled out by measuring at pH 8.6 in both buffer systems. The amylolytic activity at the particular pH values was determined as indicated above at 50° C. The results obtained are compiled in table 9 below. The activities of the two α-amylases from Streptomyces sp. B327* and B327 at pH 6.5 were set to 100% in each case; the respective values below relate to the latter.

TABLE 9
Amylolytic activity of the α-amylases from Streptomyces sp. B327*
and the Actinomycetales isolate Streptomyces sp. B327B at
alkaline pH values

| | Relative activity | Relative activity |
|---|---|---|
| | of St. sp. B327* | of St. sp. B327B |
| | α-amylase | α-amylase |
| Preincubation pH | [%] | [%] |
| 6.5 | 100 | 100 |
| 8.6 | 38 | 129 |
| 10 | 21 | 84 |
| 12 | 13 | 43 |

Compared to the α-amylase of Streptomyces sp. B327*, that of Streptomyces sp. B327 is more stable in a more alkaline medium and also has higher stability and higher amylolytic activity up to very high pH values.

Stability to Surfactants

The stability to surfactants was determined by carrying out two series of activity measurements. In the first, α-amylase was preincubated as described above with a starch substrate solution containing 0.018% (w/v) sodium dodecyl sulfate (SDS), thus resulting in a concentration of 0.01% SDS in the assay mixture after dilution. The activity was again determined at 50° C. and in 100 mM Tris-maleate buffer, pH 8.6. If these values are set in each case to 100%, both α-amylases studied have lower activities in the absence of SDS: that of the α-amylase of Streptomyces sp. B327* has an activity of 94.2% without SDS and that of Streptomyces sp. B327B has one of 89%. This shows that randomly selected representatives of the new group of α-amylases are sufficiently stable in the presence of a surfactant concentration typical for many applications.

The enzymic activity in the presence of potentially stabilizing divalent ions and, respectively, in the presence of chelators was characterized by incubating randomly selected enzymes with 3.6 mM CaCl₂ containing starch substrate solution (1% starch) or with a starch substrate solution containing 1.8 mM EDTA, thus resulting in final concentrations of 2 mM Ca²⁺ and 1 mM EDTA, respectively, in the assay carried out as described above. The remaining activity was again determined at 50° C. and 100 mM Tris-maleate buffer, pH 8.6.

If the activities of both enzymes at 2 mM CaCl₂ are set in each case to 100%, the activity of the α-amylase of Streptomyces sp. B327* decreased to 91% in the presence of 1 mM EDTA. The activity of the α-amylase of Streptomyces sp. B327B decreases to 57% in the presence of 1 mM EDTA. This shows that both enzymes have remarkably high remaining activities in the presence of surfactants and chelators. However, both enzymes react to these influences in a distinctly different and diverse manner, as indicated by the different activities in the presence of chelators.

The results of this example indicate firstly that the inventive enzymes found are not only to be regarded as α-amylases owing to their DNA and protein sequences, but indeed also have a starch-cleaving activity. The deviations of both enzymes studied with respect to their requirements on reaction conditions can be regarded as evidence of the fact that the present invention provides a broad spectrum of amylolytic enzymes with individual differences, in addition to the basic homology.

### DESCRIPTION OF THE FIGURES

FIG. 1: Position of the potentially conserved amino acid sequence blocks (“sequence anchor” A-E) in α-amylases (glycosyl hydrolase family 13, E.C. 3.2.1.1), derived from Actinomycetales α-amylase sequences, and relative positions of the PCR primers important to the invention which are listed in table 3 and indicated in the sequence listing (example 1).

The amino acid and DNA sequences comprising 567 amino acids and 1701 bp, respectively, of Streptomyces griseus α-amylase (GenBank (National Center for Biotechnology Information NCBI, National Institutes of Health, Bethesda, Md., USA), accession number X57568), having the conserved domains A to E which extend over the following amino acid positions: A: 58-91, B: 94-141, C: 155-207, D: 295-345, E: 392-427 are depicted by way of example.

FIG. 2: Size analysis of the reaction products of the inventive PCR of primer combinations GEX024/GEX026 (FIG. 2A) and GEX29/GEX031 (FIG. 2B) on streptomycete DNA (example 1).

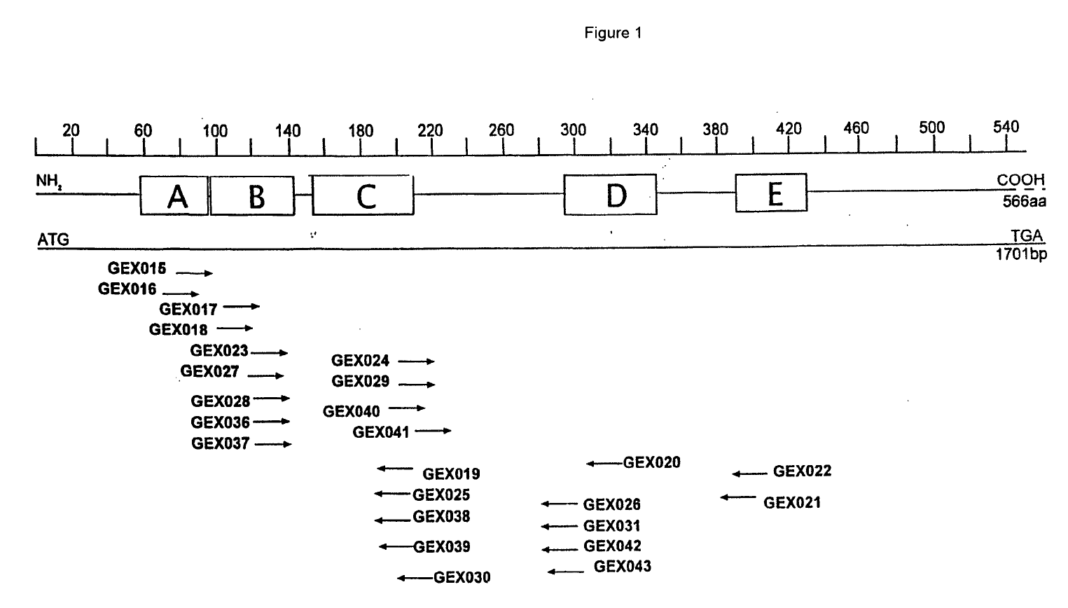

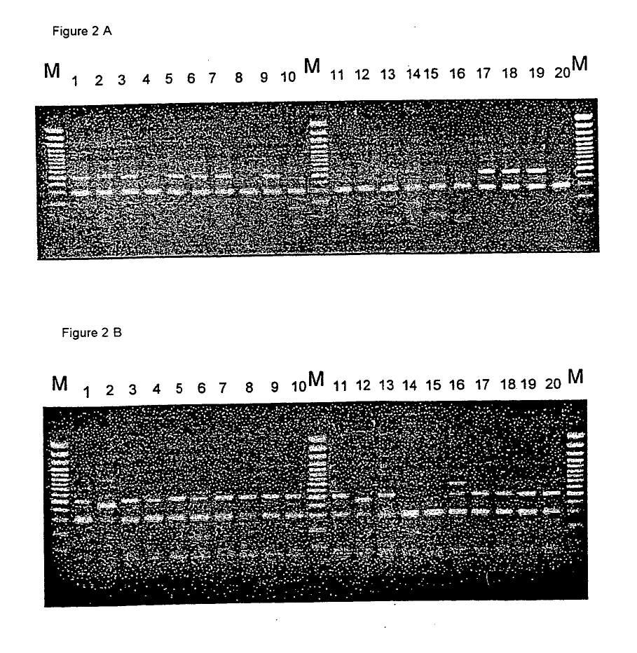

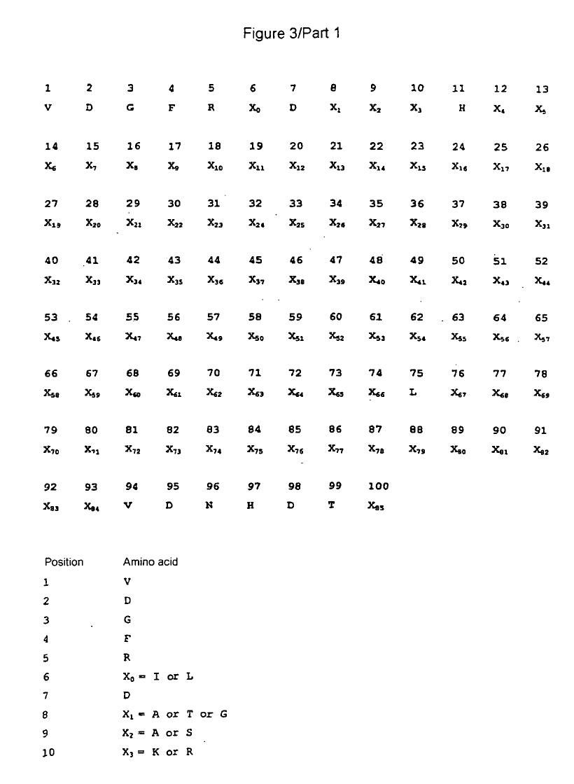

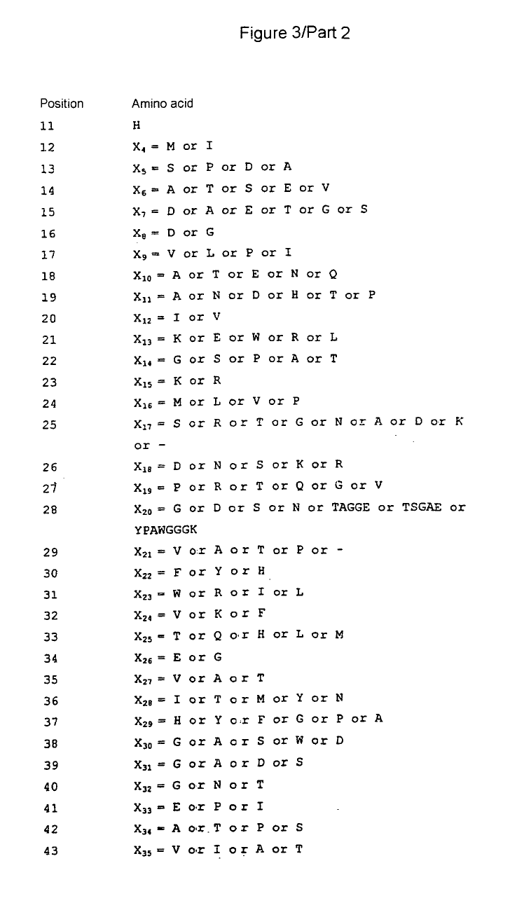

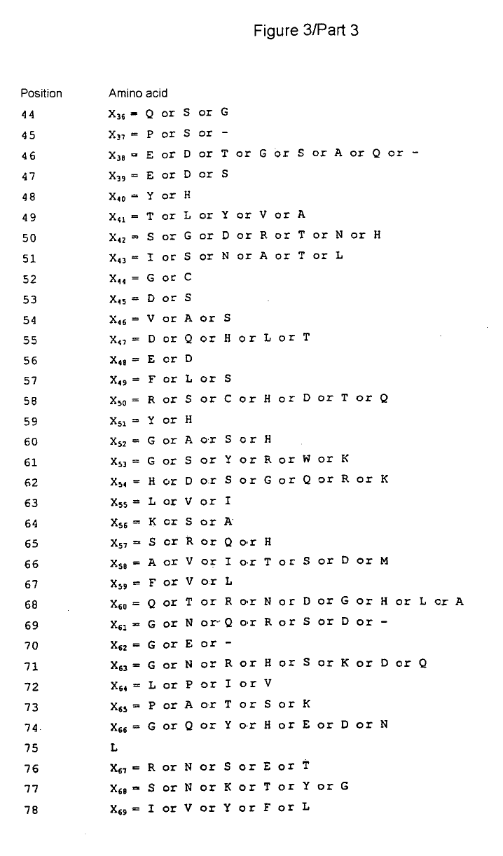

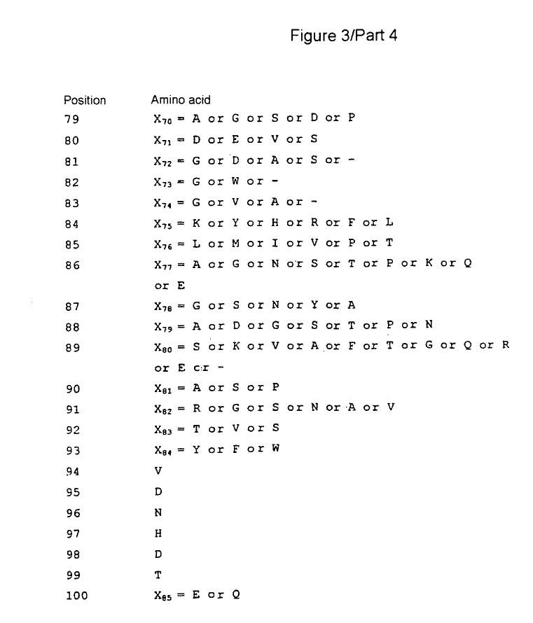

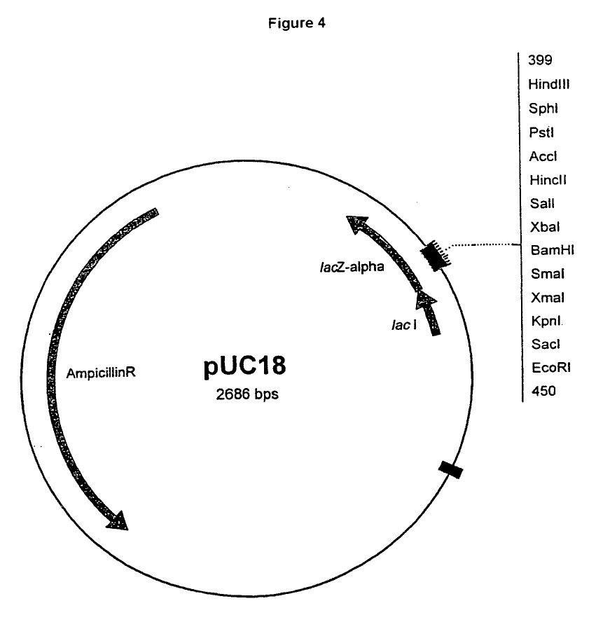

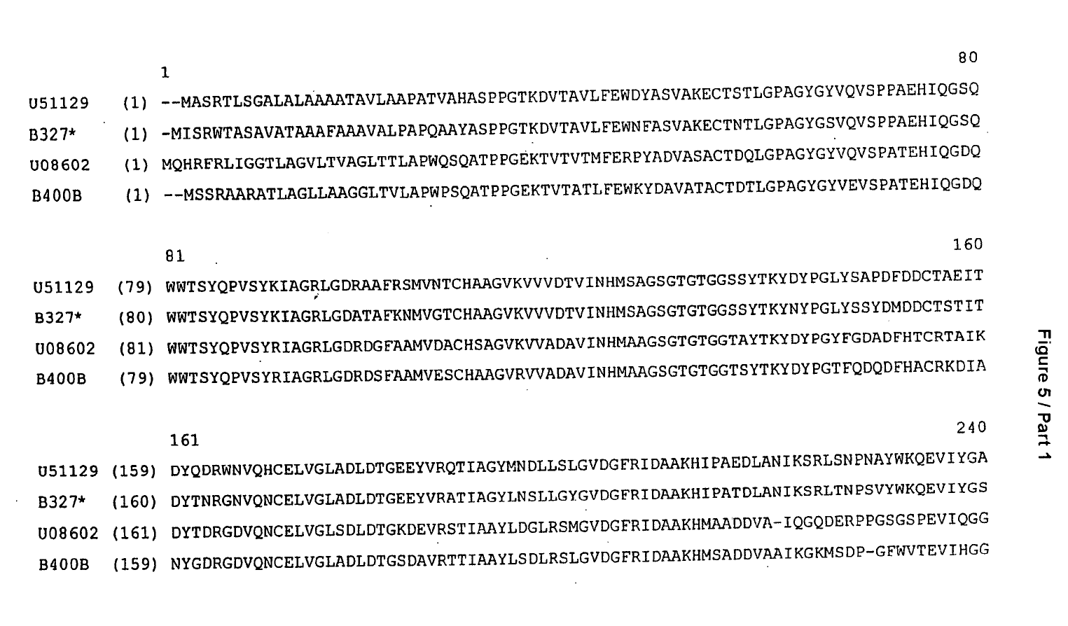

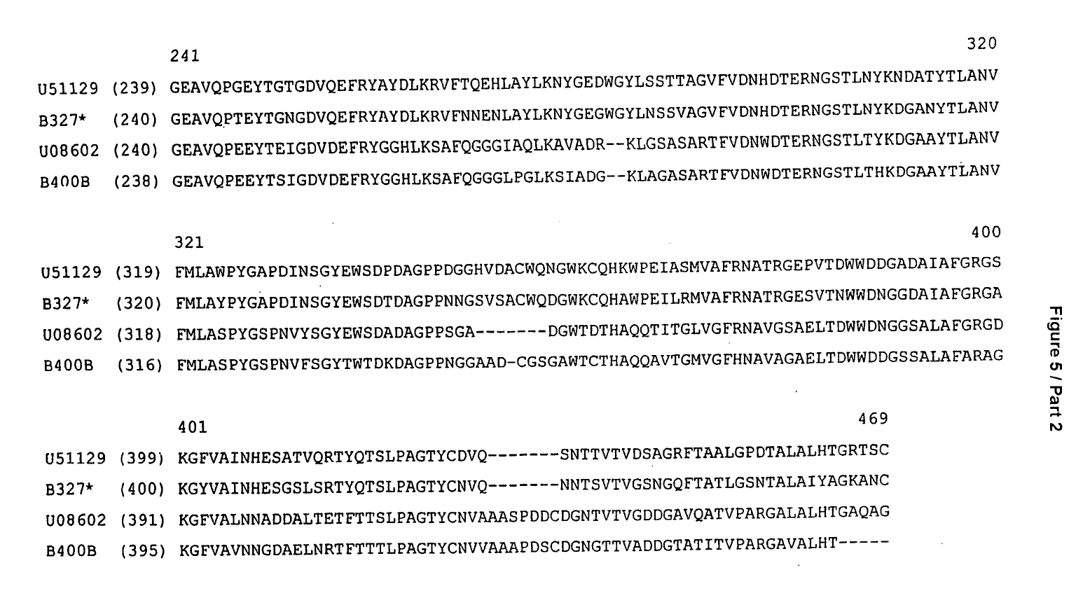

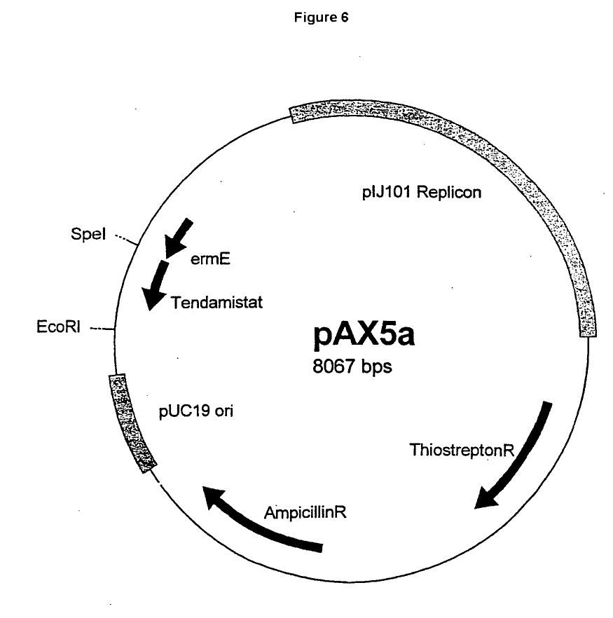

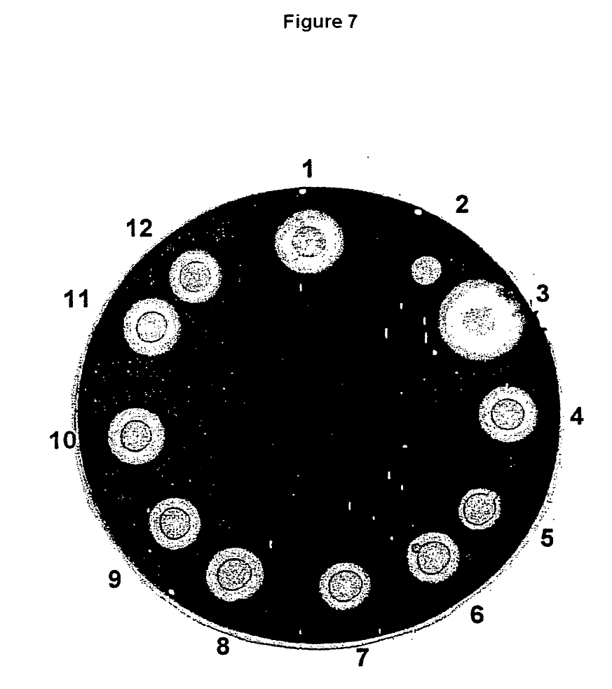

The templates used here were preparations of genomic DNA of the following 20 randomly selected Streptomyces sp. Strains:

1: B101A, 2: B114C, 3: B134, 4: B135A, 5: B138A, 6: B152A, 7: B153B, 8: B161A, 9: B156B1, 10: B157C, 11: B158A, 12: B160B, 13: B161A, 14: B373, 15: B375, 16: B380, 17: B390, 18: B392A, 19: B392A, 20: B394.

The reaction products (1/10 of reaction volume) were fractionated in each case in a 2.5% strength agarose gel; both partially present products of approx. 300 bp and approx. 500 bp in size were identified as amylase sequences by sequencing.

Markers (M) : 100 bp DNA ladder (in bp, from top to bottom: 3000, 2000, 1500, 1200, 1031, 900, 800, 700, 600, 500, 400, 300, 200, 100).

FIG. 3: Consensus sequence of α-amylases from 231 strains of the genus Streptomyces (SEQ ID NO. 2, 4 and 34 to 262) with corresponding variants for each position (example 3).

The consensus sequence is based on an alignment using the program Clustal X®, Version 1.64b (standard settings; described in: Thompson, J. D., Higgins, D. G. and Gibson, T. J. (1994), “CLUSTAL W: improving the sensitivity of progressive multiple sequence alignment through sequence weighting, position specific gap penalties and weight matrix choice”, Nucleic Acids Res., volume 22, pages 4673-4680). The amino acid positions 1-7 and 94-100 are substantially prescribed by the PCR primer sequences (GEX024/GEX026 and GEX029/031), depending on the selectivity of the PCR carried out.

This is an alternative representation of the consensus sequence indicated in SEQ ID NO. 263.

FIG. 4: Diagrammatic representation of the plasmid vector pUC18 used for expression gene banks (GenBank (National Center for Biotechnology Information NCBI, National Institutes of Health, Bethesda, Md., USA), accession number L08752; example 4).

The vector was in each case linearized with the restriction enzyme Acc I in order to receive Aci I-fragmented, genomic streptomycete DNA.

ORI: origin of replication; laci: lac-repressor gene; lacZ-alpha: gene for α-peptide of β-galactosidase; AmpicillinR: ampicillin resistance gene of 62 -lactamase.

FIG. 5: Alignment of the two complete α-amylases of the invention of Streptomyces sp. B 327* and Streptomyces sp. B400B with the most similar α-amylase sequences found in GenBank (National Center for Biotechnology Information NCBI, National Institutes of Health, Bethesda, Md., USA) on Jan. 1, 2001 (example 4).

The latter α-amylases are Streptomyces albus α-amylase (U51129) which is 80% identical to the amino acid sequence of B327 α-amylase across the length of the complete protein and Streptomyces sp. α-amylase (U08602) which correspondingly is 76.8% identical to that of Streptomyces sp. B400B (cf. tables 2 and 3).

FIG. 6: The plasmid vector pAX5a used for expressing amylases in Streptomyces lividans TK 24 (example 5).

Where:

pUC19 ori: origin of replication in E. coli; AmpicillinR, ampicillin resistance gene (beta-lactamase); ThiostreptonR, thiostreptone resistance gene; pIJ101, Replicon origin of replication in Streptomyces; ermE, ermE up promoter of S. erythrea (described in: Faβ S. H., Engels, J. W. 1996, “Influence of Specific Signal Peptide Mutations on the Expression and Secretion of the alpha-Amylase Inhibitor Tendamistat in Streptomyces lividans”, J. Biol. Chem., volume 271 (number 25), pages 15244-15252). By replacing the tendamistat sequence, genes to be cloned can be inserted via SpeI (5′ end) and EcoRI (3′ end) recognition sites into pAX5a and be expressed therein, starting from the constitutive ermE promoter.

FIG. 7: Detection of the amylolytic activity of the inventive α-amylases of Streptomyces sp. B 327* and Streptomyces sp. B400B after heterologous expression thereof in Escherichia coli.

After heterologously expressing the corresponding gene sequences in Escherichia coli (example 4), applying the transformed host cells to LB_{Amp}-agar plates containing 1% soluble starch and subsequently staining the plates by means of Lugol's solution, the colonies whose cells express the imported gene sequences can be recognized by the breakdown halos.

For this figure, samples of clones derived by isolation from the transformation mixtures were applied:

1. Positive control: expression strain containing Streptomyces griseus α-amylase in pUC18);

2. Negative control (pUC18 without DNA insert);

3. Sample of an amylase-positive clone containing genomic DNA of Streptomyces sp. B400B;

4. and 5. Samples of two genomic clones of Streptomyces sp. B327*, obtained from the same transformation;

6. to 8. Samples of three genomic clones of Streptomyces sp. B327, obtained from the same transformation;

9. to 12. Samples of four genomic clones of Streptomyces griseus, obtained from the same transformation.

1-87. (Canceled)

88. An isolated amylolytic protein comprising an amino acid sequence having at least 98% amino acid sequence identity with the consensus sequence set forth in SEQ ID NO:263.

89. The protein of claim 88 comprising an amino acid sequence having 100% amino acid sequence identity with the consensus sequence set forth in SEQ ID NO:263.

90. The protein of claim 88 comprising an amino acid sequence having at least 98% amino acid sequence identity with the subregion corresponding to positions 8 to 93 of the consensus sequence of SEQ ID NO:263.

91. The protein of claim 88 comprising an amino acid sequence having 100% amino acid sequence identity with the subregion of the consensus sequence of SEQ ID NO:263 corresponding to positions 8 to 93.

92. An isolated amylolytic protein comprising an amino acid sequence having at least 98% amino acid identity with at least one sequence selected from the group consisting of SEQ ID NOs:34-262.

93. The protein of claim 92 comprising an amino acid sequence having 100% amino acid identity with at least one sequence selected from the group consisting of SEQ ID NOs:34-262.

94. The protein of claim 92 wherein the amino acid sequence comprises the subregion corresponding to positions 8 to 93 of the consensus sequence of SEQ ID NO:263.

95. An isolated amylolytic protein comprising an amino acid sequence having at least 98% amino acid identity with at least one of the sequences indicated in SEQ ID NO: 45, 83, 97, 98, 101, 108, 109, 111, 112, 113, 115, 116, 232, 234, 236, 238, 239, or 241.

96. The protein of claim 95 comprising an amino acid sequence having 100% amino acid identity with at least one of the sequences indicated in SEQ ID NO: 45, 83, 97, 98, 101, 108, 109, 111, 112, 113, 115, 116, 232, 234, 236, 238, 239, or 241.

97. The protein of claim 95 wherein the amino acid sequence comprises the subregion corresponding to positions 8 to 93 of the consensus sequence of SEQ ID NO:263.

98. An isolated amylolytic protein comprising an amino acid sequence having at least 95% amino acid identity with at least one of the sequences indicated in SEQ ID NO: 2, 4, or 208.

99. The protein of claim 98 comprising an amino acid sequence having 100% amino acid identity with at least one of the sequences indicated in SEQ ID NO: 2, 4, or 208.

100. The protein of claim 98 wherein the amino acid sequence comprises the subregion corresponding to positions 8 to 93 of the consensus sequence of SEQ ID NO:263.

101. An isolated amylolytic protein comprising an amino acid sequence having at least 85% amino acid identity with the sequence of SEQ ID NO:6.

102. The protein of claim 101 comprising an amino acid having 100% identity with the sequence of SEQ ID NO:6.

103. An isolated amylolytic protein comprising an amino acid sequence having at least 80% amino acid identity with the sequence of SEQ ID NO:8.

104. The protein of claim 103 comprising an amino acid sequence having 100% identity with the sequence of SEQ ID NO:8.

105. An isolated amylolytic derivative of the protein of claim 88.

106. An isolated amylolytic protein or derivative thereof which shares at least one antigenic determinant with the protein of claim 88.

107. An isolated nucleic acid molecule comprising a nucleotide sequence encoding an amylolytic protein whose amino acid sequence comprises a region having at least 98% identity to the consensus sequence of SEQ ID NO:263.

108. The nucleic acid molecule of claim 10 comprising a nucleotide sequence encoding an amino acid sequence comprising a region having at least 98% identity to the subregion corresponding to positions 8 to 93 of the consensus sequence of SEQ ID NO:263.

109. An isolated nucleic acid molecule comprising a nucleotide sequence encoding an amylolytic protein whose amino acid sequence comprises a region having at least 98% identity to at least one of the amino acid sequences indicated in SEQ ID NO:34 to SEQ ID NO:262.

110. An isolated nucleic acid molecule comprising a nucleotide sequence encoding an amylolytic protein whose amino acid sequence comprises a region having at least 98% identity to at least one of the amino acid sequences indicated in SEQ ID NO: 45, 83, 97, 98, 101, 108, 109, 111, 112, 113, 115, 116, 232, 234, 236, 238, 239, or 241.

111. An isolated nucleic acid molecule comprising a nucleotide sequence encoding an amylolytic protein whose amino acid sequence comprises a region having at least 95% identity with at least one of the amino acid sequences indicated in SEQ ID NO: 2, 4, or 208.

112. An isolated nucleic acid molecule comprising a nucleotide sequence as indicated in at least one of SEQ ID NO: 1 or SEQ ID NO:3.

113. An isolated nucleic acid molecule comprising a nucleotide sequence having at least 92.5% identity with the nucleic acid sequence of SEQ ID NO:5.

114. The nucleic acid of claim 113 comprising a nucleotide sequence having 100% identity with the sequence of SEQ ID NO:5.

115. An isolated nucleic acid molecule comprising a nucleotide sequence having at least 85% identity with the nucleic acid sequence of SEQ ID NO:7.

116. The nucleic acid molecule of claim 115 comprising a nucleotide sequence having 100% identity with the nucleic acid sequence of SEQ ID NO:7.

117. An oligonucleotide comprising the nucleic acid sequence of at least one of SEQ ID NO:9 through 33.

118. A microorganism comprising the nucleic acid molecule of claim 107.

119. A PCR-based method for identifying new amylases from a plurality of organisms or nucleic acids, comprising the step of performing PCR on template material using at least one pair of primers, the pair comprising a first primer comprising a variable 3′ region and a second primer comprising a 5′ region homologous to a region of a known amylase.

120. The method of claim 119 wherein the second primer comprises a 5′ region derived from a sequence corresponding to a conserved amylase domain.

121. The method of claim 120 wherein the conserved amylase domain corresponds to at least one of amino acid positions (A) 58-91, (B) 94-141, (C) 155-207, (D) 295-345, or (E) 392-427 of Streptomyces griseus α-amylase.

122. The method of claim 120 wherein the conserved amylase domain corresponds to the β4 domain or the β7 domain of the (αβ)₈ barrel structure.

123. The method of claim 119 wherein at least one primer comprises a sequence according to one of SEQ ID NO:9-33.

124. The method of claim 123 wherein at least one primer comprises SEQ ID NO:9 and at least one primer comprises SEQ ID NO:10.

125. The method of claim 123 wherein at least one primer comprises SEQ ID NO:11 and at least one primer comprises SEQ ID NO:12.

126. The method of claim 119 wherein the template material comprises isolated nucleic acids.

127. The method of claim 126 wherein the nucleic acids are isolated from: organisms, cell cultures, strain isolates, cultured individual strains, or suitable mixtures thereof.

128. The method of claim 127 wherein the nucleic acids are isolated from microorganisms.

129. The method of claim 128 wherein the microorganisms are bacteria.

130. The method of claim 119 further comprising the steps of: obtaining at least one PCR product; introducing the PCR product into an expression gene bank; and assaying the expression gene bank for amylolytic proteins or fragments thereof by at least one of: nucleic acid hybridization, an immunochemical method or an activity assay.

131. The method of claim 130 wherein the activity assay further comprises the following steps:
a) screening a gene bank with the PCR product or a probe derived therefrom;
b) identifying at least one gene or gene fragment;
c) expressing the genes or gene fragments identified in step b);
d) obtaining at least one protein or protein fragment; and
e) measuring the contribution to an amylolytic activity assay of the proteins or protein fragments.

132. The method of claim 119 wherein PCR is carried out on a plurality of templates such that at least two different amino acid sequences are obtained having a common consensus sequence.

133. A vector comprising the nucleic acid molecule of claim 107.

134. A host cell capable of expressing the protein of claim 88 or a derivative thereof.

135. A composition comprising a detergent or cleaning agent, and the protein of claim 88 or a derivative thereof.

136. A method for the manufacture of textiles, comprising treating raw materials or intermediates previously coated with sizing with the protein of claim 88 or a derivative thereof, wherein the sizing is removed.

137. A method for starch liquefaction, wherein starch soaked in water or buffer is incubated with the protein of claim 88 or a derivative thereof, wherein the starch is cleaved into smaller molecules.

138. A method for preparing linear or short-chain oligosaccharides comprising incubating starch-like polymers with the protein of claim 88 or a derivative thereof.

139. A method for hydrolyzing cyclodextrins comprising contacting a cyclodextrin with the protein of claim 88 or a derivative thereof.

140. A method of dissolving starch-containing adhesive bonds comprising contacting the adhesive with the protein of claim 88 or a derivative thereof.

141. A method for temporary bonding comprising adding the protein of claim 88 or a derivative thereof to an adhesive, and applying the adhesive to at least one of the materials to be bonded.


SEQUENCE LISTING (273 sequences)

SEQ ID NO: 1 - DNA, 302 bp, Streptomyces sp. B327*
  CDS (2)..(301)
cgtcgacggcttccgcatcgacacggccaagcacatcccggcgaccgacctcgccaacatcaagtcgcgtctgacgaacccctccgtctactggaagcaggaggtcatctacggcagcggggaggccgtccagcccaccgagtacacgggcaacggggacgtccaggagttccgctacgcctacgacctcaagcgcgtcttcaacaacgagaacctcgcctatctgaagaactacggcgagggctgggggtacctgaacagctcggtggccggcgtctacgtcgacaaccacgacaccgagc

SEQ ID NO: 2 - protein, 100 aa, Streptomyces sp. B327*
VDGFRIDTAKHIPATDLANIKSRLTNPSVYWKQEVIYGSGEAVQPTEYTGNGDVQEFRYAYDLKRVFNNENLAYLKNYGEGWGYLNSSVAGVYVDNHDTE

SEQ ID NO: 3 - DNA, 293 bp, Streptomyces sp. B400B
  CDS (2)..(292)
cgtcgacggcttccgcatcgacgccgccaagcacatgtccgccgacgacgtcgccgcgatcaaggggaagatgagcgacccggggttctgggtcaccgaggtcatccacggcgggggagaggcggtccagccggaggagtacacctcgatcggggacgccgacgagttccgttacggcggccacctcaagtccgccttccagggcggcggcctgcccggcctgaagtcgatcgccgacggcaaactggccggcgcctccgcccgtacgtacgtggacaaccacgacaccgagc

SEQ ID NO: 4 - protein, 97 aa, Streptomyces sp. B400B
VDGFRIDAAKHMSADDVAAIKGKMSDPGFWVTEVIHGGGEAVQPEEYTSIGDADEFRYGGHLKSAFQGGGLPGLKSIADGKLAGASARTYVDNHDTE

SEQ ID NO: 5 - DNA, 1386 bp, Streptomyces sp. B327*
  CDS (1)..(1383)
  misc_feature (1)..(3): INIT_MET
gtgatatcgagatggaccgcatccgctgtcgccacggccgccgcgttcgccgccgcggtggccctgccggccccccaggccgcctacgcctccccgcccggtaccaaggacgtcaccgccgtcctcttcgagtggaacttcgcctccgtcgccaaggagtgcaccaacaccctcggccccgccggatacggctccgtgcaggtctccccgcccgccgagcacatccagggctcacagtggtggacctcgtaccagccggtcagctacaagatcgcggggcgcctcggtgacgccaccgccttcaagaacatggtcggcacctgccacgcggccggggtgaaggtcgtcgtcgacaccgtcatcaaccacatgtccgcgggcagcggcaccggtaccggcggatcgtcgtacacgaagtacaactaccccggcctgtactcctcgtacgacatggacgactgcacgtcgaccatcaccgactacaccaaccgcggcaacgtccagaactgcgaactcgtcggcctcgccgacctggacaccggcgaggagtacgtccgcgccaccatcgccggctacctgaactcgctgctcggctacggcgtcgacggcttccgcatcgacgcggccaagcacatcccggcgaccgacctcgccaacatcaagtcgcgtctgacgaacccctccgtctactggaagcaggaggtcatctacggcagcggggaggccgtccagcccaccgagtacacgggcaacggggacgtccaggagttccgctacgcctacgacctcaagcgcgtcttcaacaacgagaacctcgcctatctgaagaactacggcgagggctgggggtacctgaacagctcggtggccggcgtcttcgtcgacaaccacgacaccgagcgcaacggctccacgctcaactacaaggacggcgcgaactacaccctcgccaacgtcttcatgctcgcctacccgtacggcgccccggacatcaactcaggctacgagtggtccgacacggacgcgggcccgcccaacaacggctccgtgagcgcctgttggcaggacggctggaagtgtcagcacgcctggccggagatcctccgcatggtcgccttccgcaacgccacccgcggtgagtcggtcaccaactggtgggacaacggcggcgacgcgatcgccttcgggcggggcgccaagggctatgtcgccatcaaccacgagtccggcagcctgagccggacctaccagacctcgctgcccgccggcacgtactgcaacgtgcagaacaacacgagcgtgacggtcggctccaacggtcagttcacggccacgctcgggtccaacacggcactggcgatctacgcgggcaaggccaactgctga

SEQ ID NO: 6 - protein, 461 aa, Streptomyces sp. B327*
VISRWTASAVATAAAFAAAVALPAPQAAYASPPGTKDVTAVLFEWNFASVAKECTNTLGPAGYGSVQVSPPAEHIQGSQWWTSYQPVSYKIAGRLGDATAFKNMVGTCHAAGVKVVVDTVINHMSAGSGTGTGGSSYTKYNYPGLYSSYDMDDCTSTITDYTNRGNVQNCELVGLADLDTGEEYVRATIAGYLNSLLGYGVDGFRIDAAKHIPATDLANIKSRLTNPSVYWKQEVIYGSGEAVQPTEYTGNGDVQEFRYAYDLKRVFNNENLAYLKNYGEGWGYLNSSVAGVFVDNHDTERNGSTLNYKDGANYTLANVFMLAYPYGAPDINSGYEWSDTDAGPPNNGSVSACWQDGWKCQHAWPEILRMVAFRNATRGESVTNWWDNGGDAIAFGRGAKGYVAINHESGSLSRTYQTSLPAGTYCNVQNNTSVTVGSNGQFTATLGSNTALAIYAGKANC

SEQ ID NO: 7 - DNA, 1377 bp, Streptomyces sp. B400B
  CDS (1)..(1374)
  misc_feature (1)..(3): INIT_MET
ttgagttcccgcgccgcacgcgcaaccctggcgggcctgctcgccgccggtggcctcacggtcctcgccccctggccttcccaggcgaccccaccgggcgagaagaccgtcaccgccaccctcttcgagtggaagtacgacgcggtggccaccgcctgcaccgacaccctgggcccggccggctacggctacgtcgaggtctcgcccgccaccgagcacatccagggcgaccagtggtggacctcgtaccagccggtcagctaccggatcgcgggccgtctcggtgaccgggactccttcgccgcgatggtggagagctgccacgcggccggggtccgggtcgtggcggacgccgtgatcaaccacatggccgccggctccgggaccgggacgggcggcacgtcgtacaccaagtacgactaccccggcacgttccaggaccaggacttccacgcctgccgcaaggacatcgcgaactacggcgaccgcggcgacgtccagaactgcgaactggtcggcctcgcggacctggacaccggcagcgacgccgtacgcacgacgatcgccgcctacctctccgacctccgctccctgggcgtcgacggcttccgcatcgacgccgccaagcacatgtccgccgacgacgtcgccgcgatcaaggggaagatgagcgacccggggttctgggtcaccgaggtcatccacggcgggggagaggcggtccagccggaggagtacacctcgatcggggacgtcgacgagttccgttacggcggccacctcaagtccgccttccagggcggcggcctgcccggcctgaagtcgatcgccgacggcaaactggccggcgcctccgcccgtacgttcgtcgacaactgggacaccgagcgcaacggctccaccctcacccacaaggacggcgccgcctacaccctggccaacgtcttcatgctggcctcgccctacggctccccgaacgtcttctccggctacacctggaccgacaaggacgccggcccgccgaacggcggcgcggccgactgcggctccggcgcgtggacctgcacgcacgcccagcaggcggtcaccggcatggtcggcttccacaacgccgtcgcgggcgcggagctgaccgactggtgggacgacggctcctccgccctcgccttcgcccgcgcgggcaagggctttgtcgccgtcaacaacggcgacgcggagctgaaccgcaccttcacgacgacgctgccggcgggcacgtactgcaatgtggtcgccgcggcccccgactcctgcgacggcaacgggaccacggtcgcggacgacggcacggcgacgatcacggtcccggcgcgcggagcggtggcgctgcacacctag

SEQ ID NO: 8 - protein, 458 aa, Streptomyces sp. B400B
LSSRAARATLAGLLAAGGLTVLAPWPSQATPPGEKTVTATLFEWKYDAVATACTDTLGPAGYGYVEVSPATEHIQGDQWWTSYQPVSYRIAGRLGDRDSFAAMVESCHAAGVRVVADAVINHMAAGSGTGTGGTSYTKYDYPGTFQDQDFHACRKDIANYGDRGDVQNCELVGLADLDTGSDAVRTTIAAYLSDLRSLGVDGFRIDAAKHMSADDVAAIKGKMSDPGFWVTEVIHGGGEAVQPEEYTSIGDVDEFRYGGHLKSAFQGGGLPGLKSIADGKLAGASARTFVDNWDTERNGSTLTHKDGAAYTLANVFMLASPYGSPNVFSGYTWTDKDAGPPNGGAADCGSGAWTCTHAQQAVTGMVGFHNAVAGAELTDWWDDGSSALAFARAGKGFVAVNNGDAELNRTFTTTLPAGTYCNVVAAAPDSCDGNGTTVADDGTATITVPARGAVALHT

SEQ ID NO: 9 - DNA, 24 bp, Artificial Sequence
  PCR-Primer (GEX024)
cgtcgacggcttccgsatcgacrc

SEQ ID NO: 10 - DNA, 25 bp, Artificial Sequence
  PCR-Primer (GEX026)
gctcggtgtcgtggttgtcsacgwa

SEQ ID NO: 11 - DNA, 24 bp, Artificial Sequence
  PCR-Primer (GEX029)
cggcgtcgacggctkscgbntsga

SEQ ID NO: 12 - DNA, 25 bp, Artificial Sequence
  PCR-Primer (GEX031)
gctgggtgtcgtggttgtcsacsma

SEQ ID NO: 13 - DNA, 22 bp, Artificial Sequence
  PCR-Primer (GEX015)
ggtggacgtcctaccagccsgt

SEQ ID NO: 14 - DNA, 23 bp, Artificial Sequence
  PCR-Primer (GEX016)
gcacccgcaggagcacrycsagg

SEQ ID NO: 15 - DNA, 18 bp, Artificial Sequence
  PCR-Primer (GEX017)
cgcggccggcgtsaagrt

SEQ ID NO: 16 - DNA, 22 bp, Artificial Sequence
  PCR Primer (GEX018)
tggtcaacacctgccacgmsgc

SEQ ID NO: 17 - DNA, 24 bp, Artificial Sequence
  PCR-Primer (GEX019)
cgcggcgtcgatgckgaagmmgtc

SEQ ID NO: 18 - DNA, 26 bp, Artificial Sequence
  PCR-Primer (GEX020)
ggagccgtacggccasgcsagcatga

SEQ ID NO: 19 - DNA, 23 bp, Artificial Sequence
  PCR-Primer (GEX021)
cccttgtcgccgcgsscgaasgc

SEQ ID NO: 20 - DNA, 24 bp, Artificial Sequence
  PCR-Primer (GEX022)
ggcgtcctcgtggttgawsgcsac

SEQ ID NO: 21 - DNA, 27 bp, Artificial Sequence
  PCR-Primer (GEX023)
ggtctacgccgacgtcgtswwcaacca

SEQ ID NO: 22 - DNA, 21 bp, Artificial Sequence
  PCR-Primer (GEX025)
ggcgtcgatgcggaasccgtc

SEQ ID NO: 23 - DNA, 31 bp, Artificial Sequence
  PCR-Primer (GEX027)
gcgtccaggtctacgtcgacrysgtshtsaa

SEQ ID NO: 24 - DNA, 29 bp, Artificial Sequence
  PCR-Primer (GEX028)
caggtctacgtcgacgtcgtshtsaacca

SEQ ID NO: 25 - DNA, 27 bp, Artificial Sequence
  PCR-Primer (GEX030)
cggcggggatgtgctksrcsrmgtcsa

SEQ ID NO: 26 - DNA, 24 bp, Artificial Sequence
  PCR-Primer (GEX036)
gtacgccgacgccgtnwthaayca

SEQ ID NO: 27 - DNA, 26 bp, Artificial Sequence
  PCR-Primer (GEX037)
gtacgccgacgccgtnwthaaycaya

SEQ ID NO: 28 - DNA, 24 bp, Artificial Sequence
  PCR-Primer (GEX038)
ggcggcgtcgatcckraanccrtc

SEQ ID NO: 29 - DNA, 24 bp, Artificial Sequence
  PCR-Primer (GEX039)
cttggcggcgtcgatnckraancc

SEQ ID NO: 30 - DNA, 25 bp, Artificial Sequence
  PCR-Primer (GEX040)
tcttgctcggcgtggayggnttymg

SEQ ID NO: 31 - DNA, 24 bp, Artificial Sequence
  PCR-Primer (GEX041)
ccggatcgacgccgynaarcayat

SEQ ID NO: 32 - DNA, 26 bp, Artificial Sequence
  PCR-Primer (GEX042)
cgctcggtgtcgtggttntcnacvma

SEQ ID NO: 33 - DNA, 28 bp, Artificial Sequence
  PCR-Primer (GEX043)
cgttccgctcggtgtcgyrrttntcnac

SEQ ID NO: 34 - protein, 100 aa, Streptomyces sp. B1002
VDGFRIDAAKHMPAADLANIKSRLGDPNVYWKQEAIYGAGEAVSPDEYTGTGDVQEFRYARGLKQVFNNENLAYLKNYGEGWGFMPSSKAAVYVDNHDTE

SEQ ID NO: 35 - protein, 100 aa, Streptomyces sp. B1003B
VDGFRIDTAKHMPAADLANIKSRLGDPNVYWKQEAIYGAGEAVSPDEYTGTGDVQEFRYARGLKQVFNNENLAYLKNYGEGWGFMPSSKAAVYVDNHDTE

SEQ ID NO: 36 - protein, 97 aa, Streptomyces sp. B1006
VDGFRIDAAKHMSAADVAAVKGKMRDPGYWVQEVIYGAGEAVQPDEYTGIGDVDEFRYGSHLKSAFQGGNIAQLKSVADGKLGSDKARTYVDNHDTE

SEQ ID NO: 37 - protein, 100 aa, Streptomyces sp. B1008A1
VDGFRIDAAKHIPEDDLAAIKSRLSNPGVYWKQETIYGAGEAVQPTEYLTTGDAQEFRYSWDLKRVFTSEKLAYLKNFGEGWGYMAGGKASVYVDNHDTE

SEQ ID NO: 38 - protein, 100 aa, Streptomyces sp. B1009A
VDGFRIDAAKHIPAADLANIKSRLSNPGVYWKHEVIYGAGEAVQPTEYTGSGDVQEFRYAYDLKRVFTNENLAYLKNYGEGWGYLNSGVSGVYVDNHDTE

SEQ ID NO: 39 - protein, 100 aa, Streptomyces sp. B1010; identical to SEQ ID NO: 38

SEQ ID NO: 40 - protein, 100 aa, Streptomyces sp. B1011
VDGFRIDAAKHIPAADLANIKSRLTKPDVYWKQEVIYGAGEAVQPTEYTGNGDVQEFRYAYDLKRVFRNERLAYLKNYGEGWGYMNSSVAGVYVDNHDTE

SEQ ID NO: 41 - protein, 100 aa, Streptomyces sp. B1012B
VDGFRIDTAKHIPAADLANIKSRLTKPDVYWKQEVIYGAGEAVQPTEYTGNGDVQEFRYAYDLKRVFRNERLAYLKNYGEGWGYMNSSVAGVYVDNHDTE

SEQ ID NO: 42 - protein, 100 aa, Streptomyces sp. B1014A1
VDGFRIDAAKHIPAADLANIKSRLTKPDVYWKQEVIYGAGEAVQPTEYTGNGDVQEFRYAYDLKRVFRNERLAYLKNYGEGWGYMNSSVAGVFVDNHDTE

SEQ ID NO: 43 - protein, 100 aa, Streptomyces sp. B1017C; identical to SEQ ID NO: 42

SEQ ID NO: 44 - protein, 100 aa, Streptomyces sp. B1019
VDGFRIDAAKHIPAADLANIKSRLTKPDVYWKQEVIYGAGEAVQPTEYTGNGDVQEFRYAYDLKRVFRNEKLAYLKNYGEGWGYMNSSVAGVYVDNHDTE

SEQ ID NO: 45 - protein, 100 aa, Streptomyces sp. B101A
VDGFRIDTAKHMAAADLADIKSRLSDPDVYWKHEAIHGAGEAVSPSEYLGSGDVQEFRYGRDLKRIFTGENLAYLKNVGEAWGYMPSDKSNVFVDNHDTE

SEQ ID NO: 46 - protein, 100 aa, Streptomyces sp. B101B
VDGFRIDAAKHMPAADLANIKSRLSNPSAYWKQEVIFGAGEAVQPTEYTGNGDVQEFRYAYDLKRVFTNENLAYLKNYGEGWGYMNSSVSGVYVDNHDTE

SEQ ID NO: 47 - protein, 84 aa, Streptomyces sp. B102
GSKHMPAADIAAIKAKLNRSAYLVQEVNYGAGEPIQPTEYTGNGDVHEFRYGKDLARMFNNERLAYLRNFGESWGYLSSAKAVV

SEQ ID NO: 48 - protein, 100 aa, Streptomyces sp. B1020C
VDGFRIDAAKHIPAEDLANIKSRLSNPNAYWKQEVIYGAGEAVQPGEYTGTGDVQEFRYAYDLKRVFTNENLAYLKNYGEGWGYLNSTTAGVFVDNHDTE

SEQ ID NO: 49 - protein, 100 aa, Streptomyces sp. B1022A
VDGFRIDAAKHMAAGDLAPIKSRLSNTNVYWKQEVIHGAGEAVQPGEYTGNGDVQEFRYAYDLKRVFNNENLAYLKNYSEGWGYLNSSVSGVYVDNHDTE

SEQ ID NO: 50 - protein, 100 aa, Streptomyces sp. B1028
VDGFRIDAAKHIATEDLAAVKAKLSKPDVYWKQETIYSAGEAVQPQEYLGNGDVQEFRYARDLKRVFQSERLAYLKNYGEGWGYLPGDRASVYVDNHDTE

SEQ ID NO: 51 - protein, 100 aa, Streptomyces sp. B1029
VDGFRIDTAKHMPAGDLANIKSRLSNPGVYWKQEAIYGAGEAVSPDEYAGNGDVQEFRYARSLKQVFNNENLANLKNFGEGWGFMPSSKAAVYVDNHDTE

SEQ ID NO: 52 - protein, 100 aa, Streptomyces sp. B1030A
VDGFRIDAAKHMPAGDLANIKSRLSNPGVYWKQEAIYGAGEAVSPDEYAGNGDVQEFRYARSLKQVFNNENLANLKNFGEGWGFMPSSKAAVYVDNHDTE

SEQ ID NO: 53 - protein, 100 aa, Streptomyces sp. B1035B
VDGFRIDAAKHIAEDDLANIKSRLTDPGVYWKQETIGAAGEAVQPSEYYNTGDVQEFHYAYDLKRVFTGEKLAELKNFGEAWGYVPSGKASVFVDNHDTE

SEQ ID NO: 54 - protein, 100 aa, Streptomyces sp. B1036
VDGFRIDAAKHIAAADLANIKSRLTNPNAYWKQEVIYGAGEAVQPTEYTGNGDVQEFRYGYDLKRVFNSEKLAYLNNFGEGWGYLPGNVAGVYVDNHDTE

SEQ ID NO: 55 - protein, 100 aa, Streptomyces sp. B1037A
VDGFRIDTAKHIAAADLANIKSRLSNPNAYWKQEAIYGAGEAVSPSEYLGTGDVQEFRYARDLKRVFQNENLAYLKNYGEGWGYMASGQSAVFVDNHDTE

SEQ ID NO: 56 - protein, 100 aa, Streptomyces sp. B1039A
VDGFRIDAAKHMAAGDLAAIKSRLTNPNVYWKHEAIYGAGEAVSPSEYLGSGDVQEFRYARDLKRVFGSENLAYLKNFGESWGYMPSGQSAVYVDNHDTE

SEQ ID NO: 57 - protein, 100 aa, Streptomyces sp. B103A
VDGFRIDAAKHMPAADLANIKSRLSNPGVYWKHEAIYGAGEAVSPSEYLGSGDVQEFRYGRGLKQVFNNENLAHLKNFGEGWGFMESGKSAVFVDNHDTE

SEQ ID NO: 58 - protein, 100 aa, Streptomyces sp. B1041A1; identical to SEQ ID NO: 42

SEQ ID NO: 59 - protein, 100 aa, Streptomyces sp. B1043A
VDGFRIDAAKHMAAADLAAIKSRLSNPNVYWKHEAIYGAGETVSPTEYVGSGDVQEFRYARDLKRVFNGENLAYLKNFGEAWGHLPSDEAAVYVDNHDTE

SEQ ID NO: 60 - protein, 100 aa, Streptomyces sp. B1044C
VDGFRIDAAKHIATEDLAAVKAKLSKPDVYWKQETIYGAGEAVQPQEYLGNGDVQEFRYARDLKRVFQSERLAYLKNYGEGWGYLPGDRASVYVDNHDTE

SEQ ID NO: 61 - protein, 100 aa, Streptomyces sp. B1045
VDGFRIDTAKHIPATDLANIKSRLTNPSVYWKQEVIYGSGEAVQPTEYTGNGDVQEFRYAYDLKRVFNNENLAYLKNYGEGWGYMSSSVAGVFVDNHDTE

SEQ ID NO: 62 - protein, 100 aa, Streptomyces sp. B1046A
VDGFRIDAAKHMAAADLANIKSRLTDPNVYWKHEAIYGAGEAVSPAEYLGSGDVQEFRYARDLKRVFNNEDLAYLKNFGEAWGYLPSDQAAVFVDNHDTE

SEQ ID NO: 63 - protein, 100 aa, Streptomyces sp. B1047A1
VDGFRIDAAKHMAAADLAAIKSRLSNPNVYWKHEAMYGAGEAVSPTEYVGSGDVQEFRYARDLKRVFNGENLAYLKNFGEAWGHLPSDEAAVYVDNHDTE

SEQ ID NO: 64 - protein, 100 aa, Streptomyces sp. B1048A
  misc_feature (13)..(13): Xaa can be any naturally occurring amino acid
VDGFRIDTAKHMXAADLAAIKSRLSNPNVYWKHEAMYGAGEAVSPTEYVGSGDVQEFRYARDLKRVFNGENLAYLKNFGEAWGHLPSDEAAVFVDNHDTE

SEQ ID NO: 65 - protein, 100 aa, Streptomyces sp. B1049A; identical to SEQ ID NO: 56

SEQ ID NO: 66 - protein, 100 aa, Streptomyces sp. B1050A
VDGFRIDTAKHMAAGDLAAIKSRLTNPNVYWKHEAIYGAGEAVSPSEYLGSGDVQEFRYARDLKRVFGSENLAYLKNFGESWGYMPSGQSAVYVDNHDTE

SEQ ID NO: 67 - protein, 100 aa, Streptomyces sp. B1052A2
VDGFRIDTAKHMAAGDLANIKSRLTNPNVYWKHEAIYGAGEAVSPAEYLGSGDVQEFRYARDLKRVFNNENLAYLKNFGESWGYLPSDQAAVYVDNHDTE

SEQ ID NO: 68 - protein, 100 aa, Streptomyces sp. B1053
  misc_feature (77)..(77): Xaa can be any naturally occurring amino acid
VDGFRIDTAKHMPAADLTAIKAKVGNGSTYWKQEAIHGAGEAVQPSEYLGTGDVQEFRYARDLKRVFQNENLAHLKXFGEDWGYMASGKSAVYVDNHDTE

SEQ ID NO: 69 - protein, 97 aa, Streptomyces sp. B1059
VDGFRIDAAKHMSADDVAAIKGKMSDPGFWVTEVIHGGGEAVQPEEYTSIGDVDEFRYGGHLKSAFQGGGLPGLKSIADGKLAGASARTYVDNHDTE

SEQ ID NO: 70 - protein, 100 aa, Streptomyces sp. B1060
VDGFRIDAAKHMPAGDLANIKSRLSNPNVYWKHEAIFGAGEAVSPSEYLGSGDVQEFRYGRSLKQVFLNENLAHLKNFGEGWGFMESGKSAVYVDNHDTE

SEQ ID NO: 71 - protein, 100 aa, Streptomyces sp. B1061B
VDGFRIDTAKHMPAADLANIKSRPSNPGVYWKHEAIYGAGEAVSPSEYLGSGDVQEFRYGRGLKQVFNNENLAYLKNFGEGWGFMESGRSAVYVDNHDTE

SEQ ID NO: 72 - protein, 100 aa, Streptomyces sp. B1065
VDGFRIDTAKHMPAGDLANIKSRLSNPNVYWKHEAIFGAGEAVSPSEYLGSGDVQEFRYGRSLKQVFLNENLAHLKNFGEGWGFMESGKSAVYVDNHDTE

SEQ ID NO: 73 - protein, 100 aa, Streptomyces sp. B1067A
VDGFRIDTAKHMPAADLANIKSRLSNPGVYWKHEAIYGAGEAVSPSEYLGSGDVQEFRYGRGLKQVFNNENLAYLKNFGEGWGFMESGRSAVYVDNHDTE

SEQ ID NO: 74 - protein, 100 aa, Streptomyces sp. B1068; identical to SEQ ID NO: 72

SEQ ID NO: 75 - protein, 100 aa, Streptomyces sp. B1069B
VDGFRIDTAKHMPAADLTAIKAKVGDGGTYWKQEAIHGAGEAVQPSEYLGTGDVQEFRYARDLKRVFQNENLAHLKNFDEDWGHMQSGRSAVYVDNHDTE

SEQ ID NO: 76 - protein, 100 aa, Streptomyces sp. B106C
VDGFRIDAAKHMPAADLANIKSRLTDPGVYWKQEVIFGAGEAVQPTEYTGNGDVQEFRYAYDLKRVFNNENLAYLKNYGEGWGYLKSSSAGVYVDNHDTE

SEQ ID NO: 77 - protein, 100 aa, Streptomyces sp. B107
VDGFRIDAAKHMPAADLANIKPRLTNPSAYWKQEVIYGAGEAVQPGEYTGTGDVQEFRYAYDLKRVFNNESLAYLKNYGEGWGYLKSSSAGVYVDNHDTE

SEQ ID NO: 78 - protein, 100 aa, Streptomyces sp. B1070A
VDGFRIDTAKHIPAADLANIKSRLTNPSVYWKQEVIYGAGEAVQPTEYTGNGDVQEFRYAYDLKRVFNNENLAYLKNYGEGWGYMSSSVAGVYVDNHDTE

SEQ ID NO: 79 - protein, 100 aa, Streptomyces sp. B1071; identical to SEQ ID NO: 72

SEQ ID NO: 80 - protein, 100 aa, Streptomyces sp. B1072A
VDGFRIDAAKHIPAADLANIKSRLTNPSVYWKQEVIYGAGEAVQPTEYTGNGDVQEFRYAYDLKRVFNNENLAYLKNYGEGWGYMSSSVAGVFVDNHDTE

SEQ ID NO: 81 - protein, 100 aa, Streptomyces sp. B108
VDGFRIDAAKHMPAADLANIKSRLTNPSVFWKMEAIHGAGEAVSPSEYLGSGDVQEFRYARDLKRVLQNEKLAHLKNFGESWGYMPSGQSGVYVDNHDTE

SEQ ID NO: 82 - protein, 100 aa, Streptomyces sp. B109A
VDGFRIDAAKHMPAADLANIKSRLTKPGVYWKQEAIYGAGEAVQPSEYTGTGDVQEFRYAYDLKRVFNNENLAYLKNYGEGWGYMNSGVSGVYVDNHDTE

SEQ ID NO: 83 - protein, 100 aa, Streptomyces sp. B114C; identical to SEQ ID NO: 38

SEQ ID NO: 84 - protein, 100 aa, Streptomyces sp. B115
VDGFRIDAAKHIPAADLADIKSRLTNPSAYWKQEVIFGAGEAVQPTEYTGAGDVQEFRYAYDLKRVFNNENLAYLKNYGEGWGYLNSGSAGVFVDNHDTE

SEQ ID NO: 85 - protein, 100 aa, Streptomyces sp. B117A1
VDGFRIDAAKHIPAADLANIKSRLSNPGVYWKHEVIYGAGEAVQPTEYTGSGDVQEFSYAYDLKRVFTNENLAYLKNYGEGWGYLNSGVSGVYVDNHDTE

SEQ ID NO: 86 - protein, 100 aa, Streptomyces sp. B118
VDGFRIDAAKHMAVGDLADIKSRLGNPDVHWKHEAIHGAGEAVSPSEYLGSGDVQEFRYARDLKRVLTGGSLAHLRNFGEGWGYMASDRSNVFVDNHDTE

SEQ ID NO: 87 - protein, 100 aa, Streptomyces sp. B119E
VDGFRIDAAKHIPEDDLAAIKSRLSNQGVYWKQETIYGAGEAVQPTEYLTTGDAQELRYSWDLKRVFTSEKLAYLKNFGEGWGYMAGGKASVYVDNHDTE

SEQ ID NO: 88 - protein, 100 aa, Streptomyces sp. B120alt
VDGFRIDAAKHIPAADLANIKSRLTNPSAYWKHEVIFGAGEAVQPTEYTGTGDVQEFRYAYDLKRVFNNENLAYLKNYGEGWGYLTSGSAGVYVDNHDTE

SEQ ID NO: 89 - protein, 100 aa, Streptomyces sp. B123
  misc_feature (23)..(23): Xaa can be any naturally occurring amino acid
VDGFRIDTAKHMPAADLTAIKAXVGDGGTYWKQEAIHGAGEAVQPSEYLGTCDVQEFRYARDLKRVFQNENLAHLKNFGEDWGHMQSGRSAVFVDNHDTE

SEQ ID NO: 90 - protein, 100 aa, Streptomyces sp. B124
VDGFRIDAAKHIPAADLANIKSRLSNPGVYWKHEVIYGAGEAVQPTEYTGSGDVQEFRYAYDLKRVFTNENLAYLKNYGEGWGYLNSGVSGVFVDNHDTE

SEQ ID NO: 91 - protein, 100 aa, Streptomyces sp. B125C
VDGFRIDAAKHMAAGDLANIKSRLTNPNVYWKHEAIYGAGEAVSPAEYLGSGDVQEFRYARDLKRVFNNENLAYLKNFGESWGYLPSDQAAVYVDNHDTE

SEQ ID NO: 92 - protein, 100 aa, Streptomyces sp. B126A
VDGFRIDTAKHIPAADLADIKSRLTNPSAYWKHEVIFGAGEAVQPTEYTGAGDVQEFRYAYDLKRVFNNENLTYLKNYGEGWGYLNSGFAGVYVDNHDTE

SEQ ID NO: 93 - protein, 100 aa, Streptomyces sp. B127A
VDGFRIDTAKHMPAADLANIKSRLTKPGVYWKQEAIYGAGEAVQPSEYTGTGDVQEFRYAYDLKRVFNNENLAYLKNYGEGWGYMNSGVSGVYVDNHDTE

SEQ ID NO: 94 - protein, 100 aa, Streptomyces sp. B128B
  misc_feature (69)..(69): Xaa can be any naturally occurring amino acid
VDGFRIDTAKHIPAADLANIKSRLTNPSAYWKHEVIFGAGEAVQPTEYTGTGDVQEFRYAYDLKRVFNXENLAYLKNYGEGWGYLTSGSAGVYVDNHDTE

SEQ ID NO: 95 - protein, 100 aa, Streptomyces sp. B130B
VDGFRIDAAKHMPAADLANIKSRLTNPNAYWKLEAIHGAGEAVSPSEYLGSGDVQEFRYARDLKRVLQSEKLAHLKNFGEAWGYMPSAQSGVFVDNHDTE

SEQ ID NO: 96 - protein, 100 aa, Streptomyces sp. B131; identical to SEQ ID NO: 81

SEQ ID NO: 97 - protein, 100 aa, Streptomyces sp. B134
VDGFRIDAAKHMPAADLANIKSRLTNPNAYWKLEAIHGAGEAVSPSEYLGSGDVQEFRYARDLKRIVQSEKLAYLKNFGEAWGYMPSGQSGVYVDNHDTE

SEQ ID NO: 98 - protein, 100 aa, Streptomyces sp. B135A
VDGFRIDAAKHIPAADLANIKSRLSDPGVYWKHEVIHGAGEAVQPTEYTGSGDVQEFRYAYDLKRVFNNENLAYLKNYGEGWGYLSSGAAGVYVDNHDTE

SEQ ID NO: 99 - protein, 100 aa, Streptomyces sp. B137
VDGFRIDAAKHMPAGDPANIKSRLSNPNVYWKHEAIFGAGEAVSPSEHLGSGDVQEFRYGRSLKQVFLNENLAHLKNFGEGWGFMESGKSAVYVDNHDTE

SEQ ID NO: 100 - protein, 100 aa, Streptomyces sp. B138
VDGFRIDTAKHMPAADLANIKSRLSNPDAYWKQEVIHGAGEAVQPGEYTGNGDVQEFRYAYDLKRVFHNENLAYLKNYGEGWGYLKNSSAGVYVDNHDTE

SEQ ID NO: 101 - protein, 100 aa, Streptomyces sp. B138A; identical to SEQ ID NO: 100

SEQ ID NO: 102 - protein, 100 aa, Streptomyces sp. B138A2
VDGFRIDAAKHMPAADLANIKSRLSNPDAYWKQEVIHGAGEAVQPGEYTGNGDVQEFRYAYDLKRVFHNENLAYLKNYGEGWGYLKNSSAGVYVDNHDTE

SEQ ID NO: 103 - protein, 100 aa, Streptomyces sp. B140; identical to SEQ ID NO: 97

SEQ ID NO: 104 - protein, 100 aa, Streptomyces sp. B141; identical to SEQ ID NO: 97

SEQ ID NO: 105 - protein, 100 aa, Streptomyces sp. B142
VDGFRIDASKHMPADDLAAIKGKLANPGVYWKHEAIHGAGEAVSPSEYLGSGDVQEFRYGRGLKQVFTGGSLAHLKNFGEGWGFMESGKSAVYVDNHDTE

SEQ ID NO: 106 - protein, 100 aa, Streptomyces sp. B143
VDGFRIDAAKHMPAAGLANIKSRLTNPNAYWKLEAIHGAGEAVSPSEYLGSGDVQEFRYARDLKRIVQSEKLAYLKNFGEAWGYMPSGQSGVFVDNHDTE

SEQ ID NO: 107 - protein, 100 aa, Streptomyces sp. B148A
  misc_feature (37)..(37): Xaa can be any naturally occurring amino acid
VDGFRIDTAKHIPAADLADIKSRLTNPSAYWKQEVIXGAGEAVQPTEYTGAGDVQEFRYAYDLKRVFNNENLAYLKNYGEGWGYLNSGFAGVYVDNHDTE

SEQ ID NO: 108 - protein, 100 aa, Streptomyces sp. B152A
VDGFRIDTAKHMPAADLANIKSRLSNPGVYWKQEAIHGAGEAVSPSEYLGNGDVQEFRYARDLKRIFGSEKLSHLSTFGESWGYMASGRSGVYVDNHDTE

SEQ ID NO: 109 - protein, 100 aa, Streptomyces sp. B153 (B)
VDGFRIDTAKHMAAADLANIKSRLTDPNVYWKHEAIYGAGEAVSPAEYLGSGDVQEFRYARDLKRVFNNENLAYLKNFGEAWGYLPSDQAAVYVDNHDTE

SEQ ID NO: 110 - protein, 100 aa, Streptomyces sp. B154A
VDGFRIDAAKHMDAADLADIKSRLSNPSVYWKQEVIYGSGEAVQPTEYTSNGDVQEFRYAYDLKRVFNNENLAYLKNYGEGWGYMSSGVSGVYVDNHDTE

SEQ ID NO: 111 - protein, 100 aa, Streptomyces sp. B156B; identical to SEQ ID NO: 109

SEQ ID NO: 112 - protein, 100 aa, Streptomyces sp. B157C
VDGFRIDAAKHMDAADLADIKSRLSNPSVYWKQEVIYGSGEAVQPTEYTSNGDVQEFRYAYDLKRVFNNENLAYLKNYGEGWGYMSSGVSGVFVDNHDTE

SEQ ID NO: 113 - protein, 100 aa, Streptomyces sp. B158A
VDGFRIDTAKHMPAADLANIKSRLSNPGVYWKQEVIYGAGEAVQPTEYTGNGDVQEFRYAYDLKRVFNNENLAYLKNYGEGWGYLNSGVSGVYVDNHDTE

SEQ ID NO: 114 - protein, 100 aa, Streptomyces sp. B159
VDGFRIDAAKHIPAADLANIKSRLSNPGVYWKHEVIYGAGEAVQPTEYTGSGDVQEFRYAYDLKRVFTNENLAYLKNYGEGWGYLNSGVAGVYVDNHDTE

SEQ ID NO: 115 - protein, 100 aa, Streptomyces sp. B160B; identical to SEQ ID NO: 38

SEQ ID NO: 116 - protein, 100 aa, Streptomyces sp. B161A
VDGFRIDTAKHMAAADLANIKSRLTDPNVYWKHEAIYGAGEAVQPSEYLGTGDVQEFRYARDLKRVFQNENLAHLKNFGEDWGYMASGKSAVFVDNHDTE

SEQ ID NO: 117 - protein, 100 aa, Streptomyces sp. B166B
VDGFRIDAAKHIPAADLANIKSRLNNPSAYWKQEVIYGAGEAVQPGEYTGTGDVQEFRYAYDLKRVFNNENLAYLKNYGEGWGYLKSGSAGVYVDNHDTE

SEQ ID NO: 118 - protein, 100 aa, Streptomyces sp. B168; identical to SEQ ID NO: 38

SEQ ID NO: 119 - protein, 100 aa, Streptomyces sp. B179
VDGFRIDAAKHMPAADLANIKSRLTNPNVYWKHEAIYGAGEAVSPSEYLGSGDVQEFRYGRSLKQVFLNENLAHLKNFGEGWGFMESGKSAVYVDNHDTE

SEQ ID NO: 120 - protein, 100 aa, Streptomyces sp. B181C; identical to SEQ ID NO: 90

SEQ ID NO: 121 - protein, 100 aa, Streptomyces sp. B183B
VDGFRIDAAKHMPAADLTAIKAKVGNGSTYWKQEAIHGAGEAVQPSEYLGTGDVQEFRYARDLKRVFQNENLAYLKNFGEGWGYMASGKSAVYVDNHDTE

SEQ ID NO: 122 - protein, 100 aa, Streptomyces sp. B184
VDGFRIDAAKHMPAADLTAIKAKVGNGSTYWKQEAIHGAGEAVQPSEYLGTGDVQEFRYARDLKRVFQNGNLAYLKNFGEGWGYMASGKSAVYVDNHDTE

SEQ ID NO: 123 - protein, 100 aa, Streptomyces sp. B185 (B); identical to SEQ ID NO: 38

SEQ ID NO: 124 - protein, 100 aa, Streptomyces sp. B186A
VDGFRIDAAKHMPAADLANIKSRLTNPGVYWKHEAIYGAGEAVSPAEYLGSGDVQEFRHARDLKRVFNNENLAYLKNYGEGWGYLNSGSAGVYVDNHDTE

SEQ ID NO: 125 - protein, 100 aa, Streptomyces sp. B187A; identical to SEQ ID NO: 67

SEQ ID NO: 126 - protein, 100 aa, Streptomyces sp. B187A2; identical to SEQ ID NO: 67

SEQ ID NO: 127 - protein, 97 aa, Streptomyces sp. B194A
VDGFRIDAAKHIPASDLEAIKARMSNPNVFWVHEVIGAAGEPIQPSEYLGSGDSHEFDYARQLKRDFDGQIKNLRYIGDGKLPYDRAGVFVDNHDTE

SEQ ID NO: 128 - protein, 100 aa, Streptomyces sp. B194B1
VDGFRIDTAKHIPAADLANIKSRLSNPGVYWKHEVIYGAGEAVQPTEYTGSGDVQEFRYAYDLKRVFTNENLAYLKNYGEGWGYLNSGVSGVYVDNHDTE

SEQ ID NO: 129 - protein, 100 aa, Streptomyces sp. B196A2C
VDGFRIDAAKHIPAADLANIKSRLSNPGVYWKHEVIYGAGEAVQPTEYTGSGDVQEFRYAYDLKRVFTNENLAYLKNYGDGWGYLNSGVSGVYVDNHDTE

SEQ ID NO: 130 - protein, 100 aa, Streptomyces sp. B196B; identical to SEQ ID NO: 129

SEQ ID NO: 131 - protein, 100 aa, Streptomyces sp. B197B
  misc_feature (47)..(47): Xaa can be any naturally occurring amino acid
VDGFRIDAAKHIPAADLANIKSRLSNPGVYWKHEVIYGAGEAVQPTXYTGSGDVQEFRYAYDLKRVFTNENLAYLKNYGDGWGYLNSGVSGVFVDNHDTE

SEQ ID NO: 132 - protein, 100 aa, Streptomyces sp. B198C2
VDGFRIDAAKHIPAADLANIKSRLTNPSAYWKQEVIYGAGEAVQPTEYTGAGDVQEFRYAYDLKRVFNNENLAYLKNYGEGWGYLNSGSAGVFVDNHDTE

SEQ ID NO: 133 - protein, 100 aa, Streptomyces sp. B200B; identical to SEQ ID NO: 38

SEQ ID NO: 134 - protein, 100 aa, Streptomyces sp. B201A
VDGFRIDAAKHIPAADLADIKSRLSDPGVYWKHEVIYGAGEAVQPTEYTGSGDVQEFRYAYDLKRVFNNENLAYLKNYGEGWGYLSSGAAGVYVDNHDTE

SEQ ID NO: 135 - protein, 100 aa, Streptomyces sp. B202A
  misc_feature (55)..(55): Xaa can be any naturally occurring amino acid
VDGFRIDAAKHIPAADLANIKSRLSNPGVYWKHEVIYGAGEAVQPTEYTGSGDVXEFRYAYDLKRVFTNENLAYLKNYGEGWGYLNSGVSGVYVDNHDTE

SEQ ID NO: 136 - protein, 100 aa, Streptomyces sp. B202B; identical to SEQ ID NO: 38

SEQ ID NO: 137 - protein, 100 aa, Streptomyces sp. B206A
  misc_feature (94)..(94): Xaa can be any naturally occurring amino acid
VDGFRIDAAKHIPAADLANIKSRLSNPGVYWKHEVIYGAGEAVQPTEYTGSGDVQEFRYAYDLKRVFTNENLAYLKNYGEGWGYLNSGVSGVYXDNHDTE

SEQ ID NO: 138 - protein, 100 aa, Streptomyces sp. B207
VDGFRIDAAKHIPAADLANIKSRLSNPGVYWKHEVIYGAGEAVQPTEYTGSGDVQEFRYAYDLKRVFTNENLAYLKNYGEGWGYLNSGVAGVFVDNHDTE

SEQ ID NO: 139 - protein, 100 aa, Streptomyces sp. B208B; identical to SEQ ID NO: 66

SEQ ID NO: 140 - protein, 100 aa, Streptomyces sp. B209B; identical to SEQ ID NO: 128

SEQ ID NO: 141 - protein, 100 aa, Streptomyces sp. B210; identical to SEQ ID NO: 91

SEQ ID NO: 142 - protein, 100 aa, Streptomyces sp. B211A; identical to SEQ ID NO: 38

SEQ ID NO: 143 - protein, 100 aa, Streptomyces sp. B212B1; identical to SEQ ID NO: 38

SEQ ID NO: 144 - protein, 100 aa, Streptomyces sp. B213
VDGFRIDAAKHIPAADLANIKSRLSNRGVYWKHEVTYGAGEAVQPTEYTGSGDVQEFRYAYDLKRVFTNENLAYLKNYGEGWGYLNSGVSGVYVDNHDTE

SEQ ID NO: 145 - protein, 100 aa, Streptomyces sp. B214B
  misc_feature (13)..(13): Xaa can be any naturally occurring amino acid
VDGFRIDTAKHMXAADLANIKSRLTNPNVYWKHEAIYWAGEAVSPSEYLGSGDVQEFRYGRSLKQVFLNENLAHLKNFGEGWGFMESGKSAVFVDNHDTE

SEQ ID NO: 146 - protein, 100 aa, Streptomyces sp. B214C
VDGFRIDAAKHMPAADLANIKSRLTNPNVYWKHEAIYGAGEAVSPSEYLGSGDVQEFRYGRSLKQVFLNENLAHLKNFGEGWGFMESGKSAVFVDNHDTE

SEQ ID NO: 147 - protein, 100 aa, Streptomyces sp. B215
VDGFRIDTAKHIPAADLANIKSRLSNPGVYWKHEVIYGAGEAVQPTEYTGSGDVQEFRYAYDLKRVFTNENLAYLKNYGEGWGYLNSGVSGVFVDNHDTE

SEQ ID NO: 148 - protein, 100 aa, Streptomyces sp. B218D2
VDGFRIDAAKHIPAADLANIKSRLSNPGVYWKHEVIYGAGEAVQPTEYTGSGDVQEFRYAYDLKRVFTNENLAYLKNYGVGWGYLNSGVSGVYVDNHDTE

SEQ ID NO: 149 - protein, 100 aa, Streptomyces sp. B219A; identical to SEQ ID NO: 147

SEQ ID NO: 150 - protein, 100 aa, Streptomyces sp. B220B; identical to SEQ ID NO: 38

SEQ ID NO: 151 - protein, 100 aa, Streptomyces sp. B221A
VDGFRIDAAKHIPAADLANIKSRLSNPGVYWKHEVIYGAGEAVQPTEYTDSGDVQEFRYAYDLKRVFTNENLAYLKNYGEGWGYLNSGVSGVFVDNHDTE

SEQ ID NO: 152 - protein, 100 aa, Streptomyces sp. B222 (B)
  misc_feature (74)..(74): Xaa can be any naturally occurring amino acid
VDGFRIDAAKHMAAGDLANIKSRLTNPNVYWKHEAIYGAGEAVSPAEYLGSGDVQEFRYARDLKRVFNNENLAXLKNFGESWGYLPSDQAAVYVDNHDTE

SEQ ID NO: 153 - protein, 100 aa, Streptomyces sp. B223A
VDGFRIDAAKHMPAADLANIKSRLTNPSAYWKQEVIYGAGEAVQPGEYTGTGDVQEFRYAYDLKRVFNNENLAYLKNYGEGWGYLKSSSAGVYVDNHDTE

SEQ ID NO: 154 - protein, 100 aa, Streptomyces sp. B224 (A)
VDGFRIDAAKHIPAADLANIKSRLSSPGVYWKHEVIYGAGEAVQPTEYTGSGDVQEFRYAYDLKRVFTNENLAYLKNYGEGWGYLNSGVSGVYVDNHDTE

SEQ ID NO: 155 - protein, 100 aa, Streptomyces sp. B225B
VDGFRIDTAKHIATEDLAAVKAKLSKPDVYWKQETIYGAGEAVQSQEYLGNGDVQEFRYARDLKRVFQSERLAYLKNYGEGWGYLPGDRASVYVDNHDTE

SEQ ID NO: 156 - protein, 100 aa, Streptomyces sp. B226B; identical to SEQ ID NO: 67

SEQ ID NO: 157 - protein, 100 aa, Streptomyces sp. B227B2
VDGFRIDAAKHIATEDLAAVKAKLSKPDVYWKQETIYGAGEAVQPQEYLGNGDVQEFRYARDLKRVFQSERLAYLKNYGEGWGYLPGDRASVFVDNHDTE

SEQ ID NO: 158 - protein, 100 aa, Streptomyces sp. B228B
VDGFRIDTAKHIPAADLANIKSRLNNPSAYWKQEVIYGAGEAVQPGEYTGTGDVQEFRYAYDLKRVFNNENLAYLKNYGEGWGYLKSGSAGVYVDNHDTE

SEQ ID NO: 159 - protein, 100 aa, Streptomyces sp. B230B1; identical to SEQ ID NO: 60

SEQ ID NO: 160 - protein, 100 aa, Streptomyces sp. B231A
VDGFRIDTAKHIATEDLAAVKAKLSKPDVYWKQETIYGAGEAVQPQEYLGNGDVQEFRYARDLKRVFQSERLAYLKNYGEGWGYLPGDRASVYVDNHDTE

SEQ ID NO: 161 - protein, 100 aa, Streptomyces sp. B233C; identical to SEQ ID NO: 157

SEQ ID NO: 162 - protein, 100 aa, Streptomyces sp. B234; identical to SEQ ID NO: 38

SEQ ID NO: 163 - protein, 100 aa, Streptomyces sp. B235A; identical to SEQ ID NO: 157

SEQ ID NO: 164 - protein, 100 aa, Streptomyces sp. B237A
VDGFRIDTAKHIPAADLANIKSRLSNPGVYWKHEVIYGAGEAVQPTEYTGSGDVQEFRYAYDLKRVFTNENLAYLKNYGEGWGYLNSGVAGVFVDNHDTE

SEQ ID NO: 165 - protein, 100 aa, Streptomyces sp. B238A; identical to SEQ ID NO: 114

SEQ ID NO: 166 - protein, 100 aa, Streptomyces sp. B240A; identical to SEQ ID NO: 128

SEQ ID NO: 167 - protein, 100 aa, Streptomyces sp. B241B2; identical to SEQ ID NO: 88

SEQ ID NO: 168 - protein, 100 aa, Streptomyces sp. B242A1
VDGFRIDTAKHMPAGDPANIKSRLSNPNVYWKHEAIFGAGEAVSPSEYLGSGDVQEFRYGRSLKQVFLNENLAHLKNFGEGWGFMESGKSAVYVDNHDTE

SEQ ID NO: 169 - protein, 100 aa, Streptomyces sp. B243C
  misc_feature (93)..(93): Xaa can be any naturally occurring amino acid
VDGFRIDAAKHMPAADLANIKSRLSNPDAYWKQEVIHGAGEAVQPGEYTGNGDVQEFRYAYDLKRVFHNENLAYLKNYGEGWGYLKNSSAGVXVDNHDTE

SEQ ID NO: 170 - protein, 100 aa, Streptomyces sp. B244B2; identical to SEQ ID NO: 60

SEQ ID NO: 171 - protein, 100 aa, Streptomyces sp. B246; identical to SEQ ID NO: 114

SEQ ID NO: 172 - protein, 100 aa, Streptomyces sp. B247A; identical to SEQ ID NO: 160

SEQ ID NO: 173 - protein, 100 aa, Streptomyces sp. B248B2
VDGFRIDTAKHMPAADLANIKSRLSNPGVYWKHEAIYGAGEAVSPSEYLGSGDVQEFRYGRGLKQVFNNENLAYLKNFGEGWGFMESGKSAVYVDNHDTE

SEQ ID NO: 174 - protein, 100 aa, Streptomyces sp. B249A
VDGFRIDTAKHIATEDLAAVKAKLSKPDVYWKQETIYGAGEAVQPQEYLGNGDVQEFRYARDLKRVFQSERLAYLKNYGEGWGYLPGDRASVFVDNHDTE

SEQ ID NO: 175 - protein, 100 aa, Streptomyces sp. B249C; identical to SEQ ID NO: 37

SEQ ID NO: 176 - protein, 100 aa, Streptomyces sp. B250A2
VDGFRIDTAKHMDAADLANIKSRLSNPSVYWKQEAIYGSGEAVQPTEYTGNGDVQEFRYAYDLKRVFNNENLAYLKNYGEGWGYMSSGVSGVYVDNHDTE

SEQ ID NO: 177 - protein, 100 aa, Streptomyces sp. B251B; identical to SEQ ID NO: 147

SEQ ID NO: 178 - protein, 100 aa, Streptomyces sp. B252A
VDGFRIDTAKHMPAADVANIKSRLTNPSAYWKQEVIYGAGEAVQPGEYTGTGDVQEFRYAYDLKRVFNNENLAYLKNYGEGWGYLKSSSAGVYVDNHDTE

SEQ ID NO: 179 - protein, 100 aa, Streptomyces sp. B253
VDGFRIDTAKHIDTADLANIKSRLTNPSAYWKQEVIYGSGEAVQPTEYTRNGDVQEFRYAYDLKRVFNNENLAYLKNYGEGWGYLSSSSAGVYVDNHDTE

SEQ ID NO: 180 - protein, 100 aa, Streptomyces sp. B253A
VDGFRIDTAKHMPAADLANIKSRLTNPSAYWKQEVIYGAGEAVQPGEYTGTGDVQEFRYAYDLKRVFNNENLAYLKNYGEGWGYLKSSSAGVYVDNHDTE

SEQ ID NO: 181 - protein, 100 aa, Streptomyces sp. B255B2; identical to SEQ ID NO: 90

SEQ ID NO: 182 - protein, 100 aa, Streptomyces sp. B256A
VDGFRIDTAKHMPAADLANIKSRLTDPGVYWKQEVIFGAGEAVQPTEYTGNGDVQEFRYAYDLKRVFNNENLAYLKNYGEGWGYLKSSSAGVYVDNHDTE

SEQ ID NO: 183 - protein, 100 aa, Streptomyces sp. B259A
VDGFRIDAAKHMDAADLANIKSRLGNPSAYWKQEAIFGAGEAVQPTEYTGNGDVQEFRYGYDLKRVFTNENLAYLKNYGEGWGYMNSSVAGVYVDNHDTE

SEQ ID NO: 184 - protein, 100 aa, Streptomyces sp. B261
VDGFRIDTAKHIPAEDLANIKSRLSNPNAYWKQEVIYGAGEAVQPGEYTGTGDVQEFRYAYDLKRVFTQGNLAYLKNYGEDWGYLSSTTAGVYVDNHDTE

SEQ ID NO: 185 - protein, 100 aa, Streptomyces sp. B278
  misc_feature (94)..(94): Xaa can be any naturally occurring amino acid
VDGFRIDTAKHMAAGDLANIKSRLTNPNVYWKHEAIYGAGEAVSPAEYLGSGDVQEFRYARDLKRVFNNENLAYLKNFGESWGYLPSDQAAVYXDNHDTE

SEQ ID NO: 186 - protein, 100 aa, Streptomyces sp. B279; identical to SEQ ID NO: 38

SEQ ID NO: 187 - protein, 100 aa, Streptomyces sp. B280C
VDGFRIDAAKHMAAGDLAPIKSRLSNPNVYWKQEVIHGAGEAVQPGEYTGNGDVQEFRYAYDLKRVFNNENLAYLKNYGEGWGYLNSSVSGVFVDNHDTE

SEQ ID NO: 188 - protein, 100 aa, Streptomyces sp. B284A; identical to SEQ ID NO: 82

SEQ ID NO: 189 - protein, 100 aa, Streptomyces sp. B286A
VDGFRIDTAKHMPAADLANIKSRLSNPSVYWKQEVIFGAGEAVQPTEYTGNGDVQEFRYAYDLKRVFTNENLAYLKNYGEGWGYMNSGVSGVYVDNHDTE

SEQ ID NO: 190 - protein, 100 aa, Streptomyces sp. B287
VDGFRIDAAKHMPAGDLANIKSRLSNPNVYWKQEAIHGAGEAVSPSEYLGTGDVQEFRYGRSLKQVFLNENLAHLKNFGEGWGFMESGKSGVYVDNHDTE

SEQ ID NO: 191 - protein, 100 aa, Streptomyces sp. B292A
VDGFRIDAAKHMPAADLANIKSRLTNPNAFWKLEAIHGAGEAVSPSEYLGSGDVQEFRYARDLKRVLQNEKLAYLKNFGEAWGYMPSGQSGVYVDNHDTE

SEQ ID NO: 192 - protein, 100 aa, Streptomyces sp. B3001org
VDGFRIDTAKHIAAADLANIKSRLTNPGVYWKQEVIYGAGEAVQPTEYTGNGDVQEFCYGYDLKRVFNSEKLAYLNNFGEGWGYIPGNVAGVYVDNHDTE

SEQ ID NO: 193 - protein, 100 aa, Streptomyces sp. B3002org; identical to SEQ ID NO: 160

SEQ ID NO: 194 - protein, 100 aa, Streptomyces sp. B3003org
VDGFRIDTAKHIDTADLANIKSRLTNPGVYWKQEVIYGSGEAVQPAEYTGNGDVQEFRYAYDLKRVFTNENLAYLKNYGEGWGYLSNSVAGVYVDNHDTE

SEQ ID NO: 195 - protein, 100 aa, Streptomyces sp. B3017; identical to SEQ ID NO: 105

SEQ ID NO: 196 - protein, 100 aa, Streptomyces sp. B306
VDGFRIDAAKHMPAADLANIKSRLTNPGVYWKHEAIYGAGEAVSPAEYLGSGDVQEFRYARDLKRVFNNENLAYLKNYGEGWGYLNSGSAGVYVDNHDTE

SEQ ID NO: 197 - protein, 100 aa, Streptomyces sp. B308; identical to SEQ ID NO: 128

SEQ ID NO: 198 - protein, 100 aa, Streptomyces sp. B311
VDGFRIDAAKHMPAGDLANIKSRLTNPNVYWKHEAIYGAGEAVSPSEYLGSGDVQEFRYGRSLKQVFLNENLAHLKNFGEGWGFMESGKSAVYVDNHDTE

SEQ ID NO: 199 - protein, 100 aa, Streptomyces sp. B315; identical to SEQ ID NO: 129

SEQ ID NO: 200 - protein, 100 aa, Streptomyces sp. B317
VDGFRIDAAKHMPAADLAAIKSRLTNPNVYWKQEAIHGAGEAVSPSEYLGTGDVQEFRYGRSLKQVFLNENLAHLKNFGEGWGFMESGKSGVYVDNHDTE

SEQ ID NO: 201 - protein, 100 aa, Streptomyces sp. B318
VDGFRIDAAKHMPAGDLADIKSRLSNPNVYWKQEAIYGANEAVSPTEYLGTGDVQEFRYARGLKQTFLGGNLADLKNFGEGWGFMESGRSAVYVDNHDTE

SEQ ID NO: 202 - protein, 100 aa, Streptomyces sp. B319; identical to SEQ ID NO: 128

SEQ ID NO: 203 - protein, 100 aa, Streptomyces sp. B320A
VDGFRIDAAKHIAAADLANIKSRLTNPGVYWKQEVIYGAGEAVQPTEYTGNGDVQEFCYGYDLKRVFNSEKLAYLKNYGEGWGYMSSSVAGVYVDNHDTE

SEQ ID NO: 204 - protein, 100 aa, Streptomyces sp. B321
VDGFRIDTAKHMAVGDLADIKSRLGNPDVHWKHEAIHGAGEAVSPSEYLGSGDVQEFRYARDLKRVLTGGSLAHLRNFGEGWGYMASDRSNVYVDNHDTE

SEQ ID NO: 205 - protein, 100 aa, Streptomyces sp. B322A; identical to SEQ ID NO: 114

SEQ ID NO: 206 - protein, 100 aa, Streptomyces sp. B323
VDGFRIDAAKHMPAADLANIKTRLTNPNVYWKHEAIYGAGEAVSPSEYLGSGDVQEFRYGRSLKQVFLNENLAHLKNFGEGWGFMESGKSGVFVDNHDTE

SEQ ID NO: 207 - protein, 100 aa, Streptomyces sp. B326
VDGFRIDTAKHIPAADLANIKSRLSNPGVYWKQEAIFGAGEAVQPSEYTGTGDVQEFRYAYDLKRVFNNENLAYLKNYGEGWGYMSSGVSGVYVDNHDTE

SEQ ID NO: 208 - protein, 100 aa, Streptomyces sp. B327B
  misc_feature (5)..(5): Xaa can be any naturally occurring amino acid
VDGFXIDAAKHMPAADLTAIKAKVGDGGTYWKQEAIHGAGEAVQPSEYLGTGDVQEFRYARDLKRVFQNENLAHLKNFGEDWGHMQSGRSAVFVDNHDTE

SEQ ID NO: 209 - protein, 100 aa, Streptomyces sp. B335org; identical to SEQ ID NO: 206

SEQ ID NO: 210 - protein, 100 aa, Streptomyces sp. B345
VDGFRIDAAKHMDAADLANIKSRLSDPNVYWKQEVIHGSGEAVQPTEYTGNGDVQEFRYAYDLKRVFGNENLAYLKNYGEGWGYMNSSVAGVYVDNHDTE

SEQ ID NO: 211 - protein, 100 aa, Streptomyces sp. B346
VDGFRIDAAKHMDAADLANIKSRLSNPSVYWKQEAIYGSGEAVQPTEYTGNGDVQEFRYAYDLKRVFNNENLAYLKNYGEGWGYMNSSVSGVFVDNHDTE

SEQ ID NO: 212 - protein, 100 aa, Streptomyces sp. B347
VDGFRIDTAKHMPVADLNAIWAKLDDTTAGGEPYIFQEVYPGSTPAASDYYSAGDVLDFTYASRVKSAFQGNVSDLESLPSSGVLPPANSVSFVDNHDTE

SEQ ID NO: 213 - protein, 100 aa, Streptomyces sp. B348
VDGFRIDAARHIDTADLANIKSRLTNPNAYRKQEVIYGSGEAVQPTEYTRNGDVQEFRYAYDLKRVFNNENPAYLKNYGEGWGYLSSPSAGVYVDNHDTE

SEQ ID NO: 214 - protein, 100 aa, Streptomyces sp. B350
VDGFRIDAAKHMDAADLANIKSRLTNPSVYWKQEVIYGSGEAVQPTEYTGNGDVQEFRYAYDLKRVFNNENLAYLKNYGEGWGYMKSGVSGVYVDNHDTE

SEQ ID NO: 215 - protein, 100 aa, Streptomyces sp. B352
VDGFRIDTAKHMDAADLANIKSRLSNPSVYWKQEAIYGSGEAVQPTEYTGNGDVQEFRYAYDLKRVFNNENLAYLKNYGEGWGYMNSSVAGVYVDNHDTE

SEQ ID NO: 216 - protein, 100 aa, Streptomyces sp. B353
VDGFRIDTAKHMDAADLANIKSRLSNPSVYWKQEVIHGSGEAVQPTEYLTTGDAQEFRYSWDLKRVFTSEKLAYLKNFGEGWGYMAGGKASVFVDNHDTE

SEQ ID NO: 217 - protein, 100 aa, Streptomyces sp. B354
VDGFRIDTAKHMDAADLANIKSRLSNPSVYWKQEAIFGAGEAVQPTEYTGNGDVQEFRYGYDLKRVFNSENLAYLKNYGEGWGHMNSSVAGVYVDNHDTE

SEQ ID NO: 218 - protein, 100 aa, Streptomyces sp. B355
VDGFRIDAAKHMDAADLTNIKSRLSNPSAYWKQEAIFGAGEAVQPTEYTGNGDVQEFRYGYDLKRVFDNENLAYLRNYGEGWGYMNSSVAGVFVDNHDTE

SEQ ID NO: 219 - protein, 100 aa, Streptomyces sp. B356
VDGFRIDAAKHMDAADLANIKSRLTNPSVYWKQEVIHGAGEAVQPTEYTGNGDVQEFRYAYDLKRVFNNENLAYLKNYGEGWGYLKSSGAGVYVDNHDTE

SEQ ID NO: 220 - protein, 100 aa, Streptomyces sp. B357
VDGFRIDAAKHMAVGDLADIKSRLGSPDVHWKHEAIHGAGEAVSPSEYLGSGDVQEFRYGRSLKQVFLNENLAHLKNFGEGWGFMESGKSGVYVDNHDTE

SEQ ID NO: 221 - protein, 100 aa, Streptomyces sp. B358
VDGFRIDAAKHMPAADLAHIKSRLTRPDVYWKQEAIHGDGEAVSPGEYLGSGDVQEFRHARDLKRVFQRENLAYLKNYGEGWGYLPSAKAGVYVDNHDTE

SEQ ID NO: 222 - protein, 100 aa, Streptomyces sp. B359
VDGFRIDTAKHMDAADLANIKSRLTNPSVYWKQEVIYGSGEAVQPTEYTGNGDVQEFRYAYDLKRVFNNENLAYLRNYGEGWGYMNSGVSGVYVDNHDTE

SEQ ID NO: 223 - protein, 100 aa, Streptomyces sp. B360
VDGFRIDAAKHIDTADLANIKSRLSNPNAYWKQEVIHGSGEAVQPGEYTRNGDVQEFRYAYDLKRVFNNENLAYLKNYGEGWGYLSSSSAGVYVDNHDTE

SEQ ID NO: 224 - protein, 100 aa, Streptomyces sp. B361
VDGFRIDTAKHMPVGDLNAIRAKLDDTTSGAEPYIFQEVYPGATPAASDYYSAGDVLDFTYASRVKSAFQGNVSDLESLPSSGVLTPANSVSYVDNHDTE

SEQ ID NO: 225 - protein, 100 aa, Streptomyces sp. B362
  misc_feature (19)..(19): Xaa can be any naturally occurring amino acid
VDGFRIDAAKHMDAADLAXIKSRLSNPSVYWKQEAIYGSGEAVQPTEYTGNGDVQEFRYAYDLKRVFNNENLAYLKNYGEGWGYMSSGVSGVYVDNHDTE

SEQ ID NO: 226 - protein, 100 aa, Streptomyces sp. B363org
VDGFRIDTAKHMPAADLANIKSRLTNPSAYWKQGVIYGAGEAVQPGEYTGTGDVQEFRYAYDLKRVFNDENLAYLKNYGEGWGYLKSSSAGVYVDNHDTE

SEQ ID NO: 227 - protein, 100 aa, Streptomyces sp. B366
VDGFRIDAAKHMDAADLANIKSRLTNTSVYWKQEVIYGSGEAVQPTEYTGNGDVQEFRYAYDLKRVFNSENLAHLKNYGEGWGYMSSSVAGVYVDNHDTE

SEQ ID NO: 228 - protein, 100 aa, Streptomyces sp. B368
VDGFRIDTAKHMDTADLATIKSRLTNPNAYWKQEVIYGSGEAVQPTEYTRNGDVQEFRYAYDLKRVFNNENLAYLKNYGEGWGYMSSSVAGVYVDNHDTE

SEQ ID NO: 229 - protein, 100 aa, Streptomyces sp. B370
VDGFRIDTAKHMPAGDLANIKSRLSNPGVHWKHEAIYGAGEAVSPTEYLGSGDVQEFRYARSLKQTFNNENLANLKNFGEGWGFMESGKSAVFVDNHDTE

SEQ ID NO: 230 - protein, 100 aa, Streptomyces sp. B371
VDGFRIDTAKHMDAADLANIKSRLTNPSVYWKQEVIYGSGEAVQPTEYTGNGDVQEFRYAYDLKRVFNNENLAYLKNYGEGWGYLNSGVSGVFVDNHDTE

SEQ ID NO: 231 - protein, 100 aa, Streptomyces sp. B372
VDGFRIDTAKHMDAADLANIKSRLSNPSVYWKQEVIYGSGEAVQPTEYTSNGDVQEFRYAYDLKRVFNNENLAYLKNYGEGWGYMNSGVSGVYVDNHDTE

SEQ ID NO: 232 - protein, 100 aa, Streptomyces sp. B373
VDGFRIDTAKHMDAADLANIKSRLSNPSVYWKQEAIYGSGEAVQPTEYTGNGDVQEFRYAYDLKRVFNNENLAYLKNYGEGWGYMSSGVSGVFVDNHDTE

SEQ ID NO: 233 - protein, 100 aa, Streptomyces sp. B374B
VDGFRIDAAKHIDTADLANIKSRLTNPNAYWKQEVIYGSGEAVQPTEYTRNGDVQEFRYAYDLKRVFNNENLAYLKNYGEGWGYLSSSSAGVYVDNHDTE

SEQ ID NO: 234 - protein, 100 aa, Streptomyces sp. B375
VDGFRIDTAKHIDSADLATIKSRLSNPSVYWKQEVIYGSGEAVQPTEYTGNGDVQEFRYAYDLKRVFNNENLAYLKNYGEGWGYMSSSVAGVYVDNHDTE

SEQ ID NO: 235 - protein, 100 aa, Streptomyces sp. B376
VDGFRIDAAKHMDAADLANIKSRLTNPSVYWKQEVIHGAGEAVQPAEYTGNGDVQEFRYAYDLKRVFNNENLAYLKNYGEGWGYLKSSGAGVYVDNHDTE

SEQ ID NO: 236 - protein, 100 aa, Streptomyces sp. B380; identical to SEQ ID NO: 176

SEQ ID NO: 237 - protein, 100 aa, Streptomyces sp. B382
VDGFRIDAAKHIAAADLANIKSRLTNPGVYWKQEVIYGAGEAVQPTEYTGNGDVQEFCYGYDLKRVFNSEKLAYLNNFGEGWGYIPGNVAGVYVDNHDTE

SEQ ID NO: 238 - protein, 100 aa, Streptomyces sp. B390; identical to SEQ ID NO: 206

SEQ ID NO: 239 - protein, 100 aa, Streptomyces sp. B392A
VDGFRIDAAKHMAAADLAAIKSRLSNPNVYWKHEAIYSAGEAVSPTEYVGSGDVQEFRYARDLKRVFNGENLAYLKNFGEAWGHLPSDEAAVYVDNHDTE

SEQ ID NO: 240 - protein, 100 aa, Streptomyces sp. B393; identical to SEQ ID NO: 50

SEQ ID NO: 241 - protein, 100 aa, Streptomyces sp. B394; identical to SEQ ID NO: 157

SEQ ID NO: 242 - protein, 100 aa, Streptomyces sp. B395
VDGFRIDAAKHIDTADLANIKSRLTNPNAYWKQEVIYGSGEAVQPTEYTRNGDVQEFRYAYDLKRVFNNENLAYLKNYGEGWGYLSSTSAGVYVDNHDTE

SEQ ID NO: 243 - protein, 100 aa, Streptomyces sp. B396 (A)
VDGFRIDAAKHIDTADLANIKSRLTKPGVYWKQEAIYGAGEAVQPTEYTGNGDVQEFRYAYDLKRVFNNENLAYLKKYGEGWGYMSSSVAGVFVDNHDTE

SEQ ID NO: 244 - protein, 100 aa, Streptomyces sp. B400
  misc_feature (93)..(93): Xaa can be any naturally occurring amino acid
VDGFRIDAAKHIPAADLADIKSRLTNTSVYWKQEAIYGSGEAVQPTEYTGNGDVQEFRYAYDLKRVFNNENLAYLKNYGEGWGYMSSSVSGVXVDNHDTE

SEQ ID NO: 245 - protein, 100 aa, Streptomyces sp. B4006; identical to SEQ ID NO: 233

SEQ ID NO: 246 - protein, 103 aa, Streptomyces sp. B4006B
VDGFRIDAAKHIPEGDLQAILSRLKNVYPAWGGGKPYIFQEVIADSTISTGSYTHLGSVTESQYHRDISHAFANGNIAHLTGLGSGLTPSDKAVVYVDNHDTE

SEQ ID NO: 247 - protein, 100 aa, Streptomyces sp. B400A; identical to SEQ ID NO: 38

SEQ ID NO: 248 - protein, 100 aa, Streptomyces sp. B400A2; identical to SEQ ID NO: 2

SEQ ID NO: 249 - protein, 100 aa, Streptomyces sp. B400B3; identical to SEQ ID NO: 70

SEQ ID NO: 250 - protein, 100 aa, Streptomyces sp. B400C
VDGFRIDAAKHMPAADLTAIKAKVGDGGTYWKQEAIHGAGEAVQPSEYLGTGDVQEFRYARDLKRVFQNENLAHLKNFGEDWGHMQSGRSAVYVDNHDTE

SEQ ID NO: 251 - protein, 100 aa, Streptomyces sp. B400C2
VDGFRIDTAKHMPAADLTAIKAKVGDGGTYWKQGAIHGAGEAVQSSEYLGTGDVQEFRYARDLKRVFQNENLAHLKNFGEDWGRMQSGRSAVYVDNHDTE

SEQ ID NO: 252 - protein, 100 aa, Streptomyces sp. B400D
VDGFRIDTAKHMPAADLANIKSRLTDPGAYWKQEAIHGAGEAVSPSEYLGSGDVQEFRYARDLKRVLQNEKLAYLKNFGEAWGHMPSGRAGVYVDNHDTE

SEQ ID NO: 253 - protein, 100 aa, Streptomyces sp. B400D2
  misc_feature (8)..(8): Xaa can be any naturally occurring amino acid
VDGFRIDXAKHMPAADLANIKSRLTDPGAYWKQEAIHGAGEAVSPSEYLGSGDVQEFRYARDLKRVLQNEKLAYLKNFGEAWGHMPSGRAGVYVDNHDTE

SEQ ID NO: 254 - protein, 100 aa, Streptomyces sp. B400E; identical to SEQ ID NO: 72

SEQ ID NO: 255 - protein, 100 aa, Streptomyces sp. B400G
VDGFRIDAAKHIPAADLANIESRLSNPGVYWKHEVIYGAGEAVQPTEYTGSGDVQEFRYAYDLKRVFTNENLAYLKNYGEGWGYLNSGVSGVYVDNHDTE

SEQ ID NO: 256 - protein, 100 aa, Streptomyces sp. B400G2
  misc_feature (8)..(8): Xaa can be any naturally occurring amino acid
VDGFRIDXAKHIPAADLANIKSRLSNPGVYWKHEVIYGAGEAVQPTEYTGSGDVQEFRYAYDLKRVFTNENLAYLKNYGEGWGYLNSGVSGVYVDNHDTE

SEQ ID NO: 257 - protein, 100 aa, Streptomyces sp. B400I
VDGFRIDTAKHMPAADLANIKSRLTNPNVYWKHEAIYGAGEAVSPSEYLGSGDVQEFRYGRSLKQVFLNENLAHLKNFGEGWGFMESGKSAVYVDNHDTE

SEQ ID NO: 258 - protein, 100 aa, Streptomyces sp. B400J
VDGFRIDAAKHMPVADLNAIWAKLDNTTSGAEPYIFQEVYPGSTPAASDYYSAGDVLDFSYASKVKSSFQGNISDLESLPSSGALTPANSVSYVDNHDTE

SEQ ID NO: 259 - protein, 100 aa, Streptomyces sp. B400K; identical to SEQ ID NO: 258

SEQ ID NO: 260 - protein, 100 aa, Streptomyces sp. B400L; identical to SEQ ID NO: 258

SEQ ID NO: 261 - protein, 100 aa, Streptomyces sp. B402
VDGFRIDAAKHMPAADLANIKSRLTDPGAYWKQEAIHGAGEAVSPSEYLGSGDVQEFRYARDLKRVLQNEKLAYLKNFGEAWGHMPSGRAGVYVDNHDTE

SEQ ID NO: 262 - protein, 100 aa, Streptomyces sp. B907
VDGFRIDAAKHMPAADLANIKTRLANPNVYWKHEAIYGAGEAVSPSEYLGSGDVQEFRYGRSLKQVFLNENLAHLKNFGEGWGFMESGKPGVYVDNHDTE

SEQ ID NO: 263 - protein, 100 aa, Artificial Sequence
  Synthetic Construct
VDGFRXDXXXHXXXXXXXXXXXXXXXXXXXXXXXXXXXXXXXXXXXXXXXXXXXXXXXXXXXXXXXXXXXXXXXLXXXXXXXXXXXXXXXXXXVDNHDTX

SEQ ID NO: 264 - DNA, 20 bp, Artificial Sequence
  Primer
gctcggatccactagtaacg

SEQ ID NO: 265 - DNA, 19 bp, Artificial Sequence
  Primer
ctctagatgcatgctcgag

SEQ ID NO: 266 - DNA, 16 bp, Artificial Sequence
  M13 Forward Primer
gtaaaacgacggccag

SEQ ID NO: 267 - DNA, 17 bp, Artificial Sequence
  M13 Reverse Primer
caggaaacagctatgac

SEQ ID NO: 268 - DNA, 35 bp, Artificial Sequence
  Forward Primer
aaaactagtaaggagaacccccacatgatatcgag

SEQ ID NO: 269 - DNA, 32 bp, Artificial Sequence
  Reverse Primer
gaattcgccctttcagcagttggccttgcccg

SEQ ID NO: 270 - protein, 458 aa, Streptomyces albus
MASRTLSGALALAAAATAVLAAPATVAHASPPGTKDVTAVLFEWDYASVAKECTSTLGPAGYGYVQVSPPAEHIQGSQWWTSYQPVSYKIAGRLGDRAAFRSMVNTCHAAGVKVVVDTVINHMSAGSGTGTGGSSYTKYDYPGLYSAPDFDDCTAEITDYQDRWNVQHCELVGLADLDTGEEYVRQTIAGYMNDLLSLGVDGFRIDAAKHIPAEDLANIKSRLSNPNAYWKQEVIYGAGEAVQPGEYTGTGDVQEFRYAYDLKRVFTQEHLAYLKNYGEDWGYLSSTTAGVFVDNHDTERNGSTLNYKNDATYTLANVFMLAWPYGAPDINSGYEWSDPDAGPPDGGHVDACWQNGWKCQHKWPEIASMVAFRNATRGEPVTDWWDDGADAIAFGRGSKGFVAINHESATVQRTYQTSLAGTYCDVQSNTTVTVDSAGRFTAALGDTALALHTGRTSC

SEQ ID NO: 271 - protein, 461 aa, Streptomyces sp. B327*
MISRWTASAVATAAAFAAAVALPAPQAAYASPPGTKDVTAVLFEWNFASVAECTNTLGPAGYGSVQVSPPAEHIQGSQWWTSYQPVSYKIAGRLGDATAFKNMVGTCHAAGVKVVVDTVINHMSAGSGTGTGGSSYTKYNYPGLYSSYDMDDCTSTITDYTNRGNVQNCELVGLADLDTGEEYVRATIAGYLNSLLGYGVDGFRIDAAKHISPATDLANIKSRLTNPSVYWKQEVIYGSGEAVQPTEYTGNGDVQEFRYAYDLKRVFNNENLAYLKNYGEGWGYLNSSVAGVFVDNHDTERNGSTLNYKDGANYTLANVFMLAYPYGAPDINSGYEWSDTDAGPPNNGSVSACWQDGWKCQHAWPEILRMVAFRNATRGESVTNWWDNGGDAIAFGRGAKGYVAINHESGSLSRTYQTSLPAGTYCNVQNNTSVTVGSNGQFTATLGSNTALAIYAGKANC

SEQ ID NO: 272 - protein, 457 aa, Streptomyces sp.
MQHRFRLIGGTLAGVLTVAGLTTLAPWQSQATPPGEKTVTVTMFERPYADVASACTDQLGPAGYGYVQYSPATEHIQGDQWWTSYQPVSYRIAGRLGDRDGFAAMVDACHSAGVKVVADAVINHMAAGSGTGTGGTAYTKYDYPGYFGDADFHTCRTAIKDYTDRGDVQNCELVGLSDLDTGKDEVRSTIAAYLDGLRSMGVDGFRIDAAKHMAADDVAIQGQDERPPGSGSPEVIQGGGEAVQPEEYTEIGDVDEFRYGGHLKSAFQGGGIAQLKAVDRKLGSASARTFVDNWGTERNGSTLTYKDGAAYTLANVFMLASPYGSNVYSGYEWSDADAGPPSGADGWTDTHAQQTITGLVGFRNAVGSAELTDWWDNGGSALAFGRGDKGFVALNNADDALTETFTTSLPAGTYCNVAAASPDDCDGNTVTVGDDGAVQATVPARGALALHTGAQAG

SEQ ID NO: 273 - protein, 456 aa, Streptomyces sp. B400B
MSSRAARATLAGLLAAGLTVLAPWPSQATPPGEKTVTATLFEWKYDAVATACTDTLGPAGYGYVEVSPATEHIQGDQWWTSYQPVSYRIAGRLGDRDSFAAMVESCHAAGVRVVADAVINHMAAGSGTGTGGTSYTKYDYPGTFQDQDFHACRKDIANYGDRGDVQNCELVGLADLDTGSDAVRTTIAAYLSDLRSLGVDGFRIDAAKHMSADDVAAIKGKMSDPGFWVTEVIHGGGEAVQPEEYTSIGDVDEFRYGGHLKSAFQGGGLPGLKSIADGKLAGASARTFVDNWDTERNGSTLTHKDGAAYTLANVFMLASYGSPNVFSGYTWTDKDAGPPNGGAADCGSGAWTCTHAQQAVTGMVGFHNAVAGAELTDWWDDGSSALAFARAGKGFVAVNNGDAELNRTFTTTLPAGTYCNVVAAAPDSCDGNGTTVADDGTATITVPARGAVALHT